(19) United States
(12) Patent Application Publication
(10) Pub. No.: US 2025/0011365 A1
(43) Pub. Date: Jan. 9, 2025

(54) ECHINOCANDIN-BASED DRUG, AND PREPARATION METHOD THEREFOR AND USE THEREOF

(71) Applicant: Shenzhen Sungening Biological Co., Ltd, Shenzhen (CN)

(72) Inventors: Yiguo ZHOU, Shenzhen (CN); Haoyu ZHANG, Shenzhen (CN); Conghui PENG, Shenzhen (CN); Hanlin WEI, Shenzhen (CN); Shenghong HUANG, Shenzhen (CN); Renchong YAO, Shenzhen (CN); Chester Chenguang YUAN, Shenzhen (CN)

(21) Appl. No.: 18/399,726

(22) Filed: Dec. 29, 2023

(30) Foreign Application Priority Data
Jun. 13, 2023 (CN) 202310697710.3
Jun. 15, 2023 (CN) 202310711545.2

(51) Int. Cl. C07K 7/56; A61P 31/10; C07C 65/28; C07D 213/65; C07D 215/48; C07D 239/34

(52) U.S. Cl. CPC C07K 7/56; A61P 31/10; C07C 65/28; C07D 213/65; C07D 215/48; C07D 239/34; A61K 38/00

(57) ABSTRACT

The present invention discloses a new compound, such as a compound of formula I, or a pharmaceutically acceptable salt thereof or an isomer thereof. The compound has a good fungal inhibitory effect, can be used for effectively preventing or treating fungal infections, and is a new generation of echinocandin-based drugs with higher efficiency.

### TECHNICAL FIELD

The prevent invention relates to an echinocandin-based drug, and belongs to the field of medical chemistry.

### BACKGROUND ART

Fungal infections can be divided into 4 categories according to the parts, of the human body where fungal infection occurs: superficial fungal infections, skin fungal infections, subcutaneous fungal infections and systemic fungal infections, in which the subcutaneous fungal infections and systemic fungal infections are also known as invasive fungal infections. Fungi can not only invade human skin, mucous membrane, deep tissues and internal organs, but also cause systemic disseminated infections with a high mortality rate. In 2022, WHO published the Fungal Priority Pathogens List to Guide Research, Development and Public Health Action, which listed common Aspergillus fumigatus, Cryptococcus neoformans, Candida albicans and Candida auris in clinic as critical priority pathogens.

Invasive fungal infections are an important cause of the persistently high morbidity and mortality, and these infections are mostly found in high-risk populations such as those with immunodeficiency, organ or bone marrow transplantation, and severe SARS-CoV-2 infection. At present, the antifungal drugs available in the market are very limited, mainly including triazole-based antifungal drugs, polyene-based antifungal drugs and echinocandin-based antifungal drugs. In addition, the continuous emergence of drug-resistant strains aggravates the challenges posed by invasive fungal infections, so it is particularly urgent to develop a variety of new antifungal agents to meet the current clinical needs.

β-(1,3)-D-glucan is an important component for constituting the fungal cell wall, and is mainly synthesized via enzymes located in the cell membrane. Echinocandin-based drugs can non-competitively inhibit β-(1,3)-D-glucan synthase and interfere with the synthesis of β-(1,3)-D-glucan so as to destroy the integrity of a fungal cell wall, resulting in the lysis and death of fungal cells. Echinocandin-based drugs are the first-line drugs for the treatment of invasive candidiasis, which can directly act on the cell wall of fungi. Since human cells do not contain cell walls, these drugs have low toxicity and are safe to human body.

Caspofungin is the earliest echinocandin-based drug for treating fungal infections in clinic, which can effectively treat candidiasis and invasive aspergillosis that is ineffective or intolerant to other treatments. The drug is only administered by injection, with poor flexibility. The drug is mostly used in the early stage, and oral preparations such as voriconazole are mainly used in maintenance treatment in the late stage. In addition, the drug needs to be stored at 2° C.-8° C., and have certain requirements for production capacity.

In March 2023, rezafungin was approved by the FDA for the treatment of candidal bacteremia and invasive candidiasis.

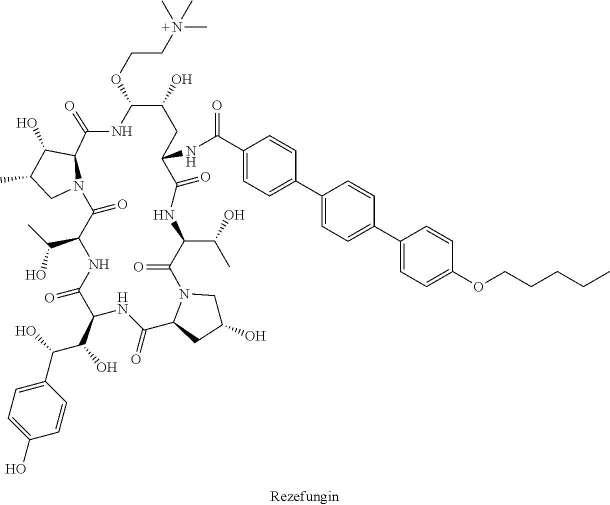

Compared with previous generations of drugs, rezafungin has a longer half-life, significantly reducing the frequency of administration over the treatment cycle. However, the recommended dose of this drug is large, and after administration, patients will experience side effects such as abdominal pain, diarrhea, nausea, vomiting and constipation. Therefore, it is of great significance to develop a new generation of echinocandin-based drugs with higher efficiency.

### SUMMARY OF THE INVENTION

In order to solve the above problems, the present invention provides a new compound, such as a compound as shown in formula I, or a pharmaceutically acceptable salt thereof or an isomer thereof,

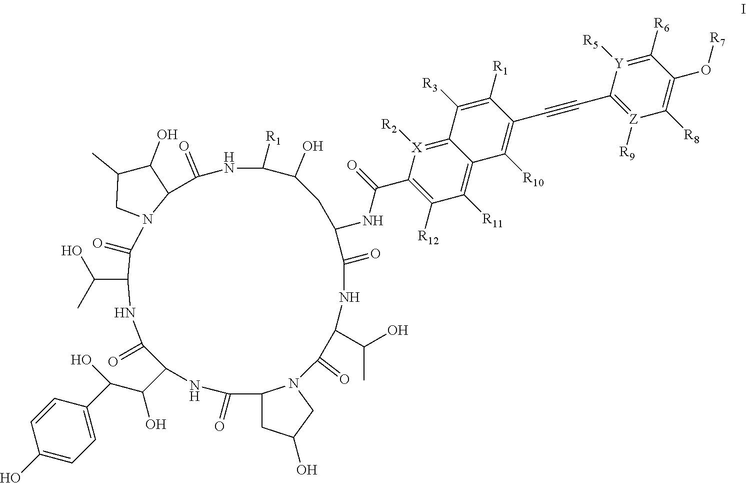

X, Y and Z are each independently selected from C and N;
R₂, R₃, R₄, R₅, R₆, R₈, R₉, R₁₀, R₁₁ and R₁₂ are each independently selected from hydrogen, deuterium, halogen, cyano, thiocyano, isothiocyano and lower alkyl;
R₇ is selected from C₁₋₁₀ lower alkyl, C₂₋₁₀ alkenyl, C₂₋₁₀ alkynyl, aryl, heteroaryl, cyclic hydrocarbonyl and heterocyclyl;
R₁ is selected from hydroxyl, hydrogen, deuterium, halogen, cyano, thiocyano, isothiocyano, O[C(R_{A1})(R_{A2})]ₐ[C(R_{A3})(R_{A4})]ⱼX₁, NH[C(R_{A1})(R_{A2})]ₐ[C(R_{A3})(R_{A4})]ⱼX₁, O(CH₂CH₂O)_{b}CH₂CH₂X₁, O(CH₂CH₂CH₂O)_{b}CH₂CH₂X₁, O(CH₂CH₂NH)_{b}CH₂CH₂X₁, NH(CH₂CH₂O)_{b}CH₂CH₂X₁, NH(CH₂CH₂NH)_{b}CH₂CH₂X₁, NH(CH₂CH₂CH₂O)_{b}CH₂CH₂X₁, NH[(CH₂(CH₂)_{c}O)]_{b}CH{CH₂[OCH₂(CH₂)_{c}]_{d}X₁}₂, O[(CH₂(CH₂)_{c}O)]_{b}CH{CH₂[OCH₂(CH₂)_{c}]_{d}X₁}₂ and (OCH₂CH₂)_{b}(NHCH₂CH₂)ₑX₂,
R_{A1}, R_{A2}, R_{A3} and R_{A4} are independently selected from hydrogen, deuterium, halogen, lower alkyl, cyclic hydrocarbonyl and cyclic hydrocarbylene

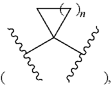

X₁ is independently N(R_{C1}R_{C2}R_{C3}) or the following structure

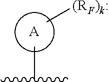

ring A is an optionally substituted, saturated or unsaturated monocyclic ring or fused ring containing one or more N atoms,

R_{C1}, R_{C2} and R_{C3} are independently selected from H, C₁₋₆ alkyl, halogenated C₁₋₆ lower alkyl and deuterated C₁₋₆ lower alkyl, and at least one of R_{C1}, R_{C2} and R_{C3} is not hydrogen,

each R_{F} is independently selected from H, deuterium, hydroxyl, hydroxylalkyl, amino, alkoxy, lower alkyl, alkenyl, alkynyl, halogen, SR′, SOR′, SO₂R′, NR′(R″), COOR′ and CONR′(R″), wherein the lower alkyl is optionally substituted with one or more substituents selected from deuterium, alkyl, cycloalkyl, alkoxy, hydroxylalkyl, alkenyl, alkynyl, aryl, heteroaryl, nitro, a nitrile group, hydroxyl, halogen, SR′, NR′(R″), COOR′ and CONR′(R″),

X₂ is N(R_{D1}R_{D2}R_{D3}) or the structure of X₁,

R_{D1}, R_{D2} and R_{D3} are independently selected from H, C₁₋₆ lower alkyl, halogenated C₁₋₆ lower alkyl and deuterated C₁₋₆ lower alkyl,

R′ and R″ are independently selected from hydrogen, hydroxyl, alkyl, alkoxy, alkenyl and —C(O)R_{J},

R_{J} is selected from hydrogen, deuterium, C₁₋₁₀ lower alkyl, cyclic hydrocarbonyl and cyclic hydrocarbylene,

and a is an integer of 0-5, b is an integer of 1-5, c is an integer of 1-2, d is an integer of 0-3, e is an integer of 1-5, k is an integer of 0-20, j is an integer of 0-5, and n is an integer of 1-7.

Further, R₁ is selected from O(C(R_{A1})(R_{A2}))ₐ(C(R_{A3})(R_{A4}))ⱼX₁, NH(C(R_{A1})(R_{A2}))ₐ(C(R_{A3})(R_{A4}))ⱼX₁, O(CH₂CH₂O)_{b}CH₂CH₂X₁, O(CH₂CH₂CH₂O)_{b}CH₂CH₂X₁, O(CH₂CH₂NH)_{b}CH₂CH₂X₁, NH(CH₂CH₂O)_{b}CH₂CH₂X₁, NH(CH₂CH₂NH)_{b}CH₂CH₂X₁, NH(CH₂CH₂CH₂O)_{b}CH₂CH₂X₁, NH[(CH₂(CH₂)_{c}O)]_{b}CH{CH₂[OCH₂(CH₂)_{c}]_{d}X₁}₂, O[(CH₂(CH₂)_{c}O)]_{b}CH{CH₂[OCH₂(CH₂)_{c}]_{d}X₁}2 and (OCH₂CH₂)_{b}(NHCH₂CH₂)ₑX₂,

R_{A1}, R_{A2}, R_{A3} and R_{A4} are independently selected from hydrogen, deuterium, halogen, lower alkyl, cyclic hydrocarbonyl and cyclic hydrocarbylene

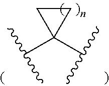

X₁ is independently N(R_{C1}R_{C2}R_{C3}) or the following structure

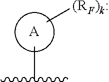

ring A is an optionally substituted, saturated or unsaturated monocyclic ring or fused ring containing one or more N atoms,

R_{C1}, R_{C2} and R_{C3} are independently selected from H, halogenated C₁₋₆ lower alkyl and deuterated C₁₋₆ lower alkyl, and at least one of R_{C1}, R_{C2} and R_{C3} is not hydrogen,

each R_{F} is independently selected from H, deuterium, hydroxyl, hydroxylalkyl, amino, alkoxy, lower alkyl, alkenyl, alkynyl, halogen, SR′, SOR′, SO₂R′, NR′(R″), COOR′ and CONR′(R″), wherein the lower alkyl is optionally substituted with one or more substituents selected from deuterium, alkyl, cycloalkyl, alkoxy, hydroxylalkyl, alkenyl and alkynyl,

X₂ is N(R_{D1}R_{D2}R_{D3}) or the structure of X₁,

R_{D1}, R_{D2} and R_{D3} are independently selected from H, C₁₋₆ lower alkyl, halogenated C₁₋₆ lower alkyl and deuterated C₁₋₆ lower alkyl,

R′ and R″ are independently selected from hydrogen, hydroxyl, alkyl, alkoxy, alkenyl and —C(O)R_{J},

R_{J} is selected from hydrogen, C₁₋₁₀ lower alkyl, cyclic hydrocarbonyl and cyclic hydrocarbylene,

and a is an integer of 0-5, b is an integer of 1-5, c is an integer of 1-2, d is an integer of 0-3, e is an integer of 1-5, k is an integer of 0-20, j is an integer of 0-5, and n is an integer of 1-7.

Still further, X₁ is selected from the following structures:

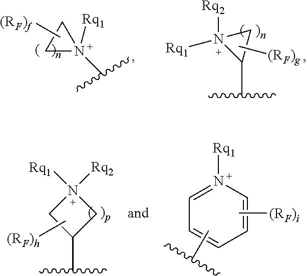

each R_{F} is independently selected from H, deuterium, hydroxyl, hydroxylalkyl, amino, alkoxy, lower alkyl, alkenyl, alkynyl, halogen, SR′, SOR′, SO₂R′, NR′(R″), COOR′ and CONR′(R″), wherein the lower alkyl is optionally substituted with one or more substituents selected from deuterium, alkyl, cycloalkyl, alkoxy, hydroxylalkyl, alkenyl and alkynyl,

R_{q1} and R_{q2} are independently H or C₁₋₆ lower alkyl, the lower alkyl is optionally substituted with one or more substituents selected from deuterium, alkyl, cycloalkyl, alkoxy, hydroxylalkyl, alkenyl, alkynyl, aryl, heteroaryl, nitro, a nitrile group, hydroxyl, halogen, SR′, NR′(R″), COOR′ and CONR′(R″),

R′ and R″ are independently selected from hydrogen, hydroxyl, alkyl, alkoxy, alkenyl and —C(O)R_{J},

R_{J} is selected from hydrogen, deuterium, C₁₋₁₀ lower alkyl, cyclic hydrocarbonyl and cyclic hydrocarbylene,

and f is an integer of 0-16, g is an integer of 0-16, h is an integer of 0-9, i is an integer of 0-4, n is an integer of 1-7, and p is an integer of 1-3.

In the present invention, R₁ is selected from hydroxyl, hydrogen, deuterium or one of the following structures:

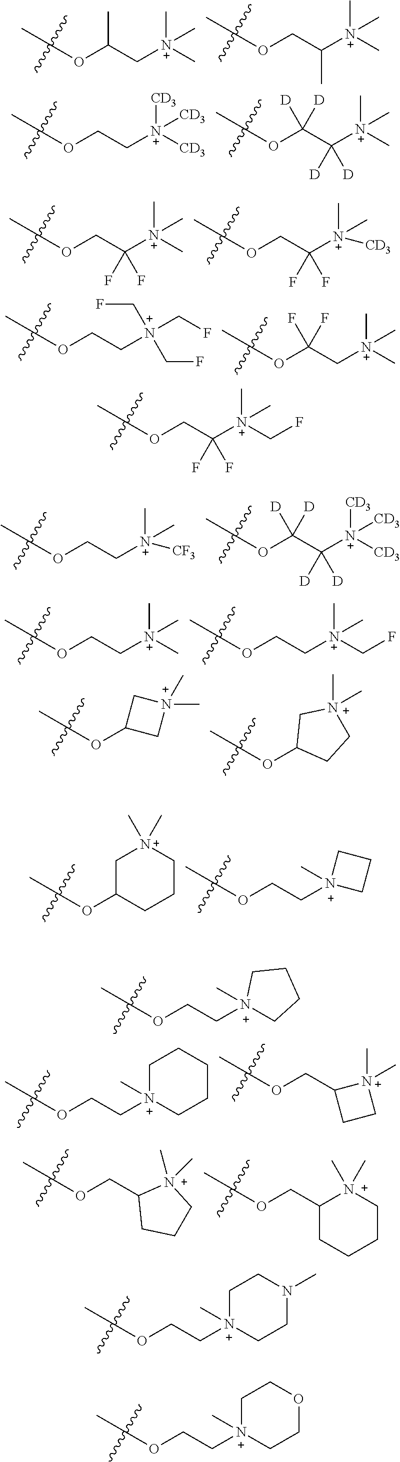

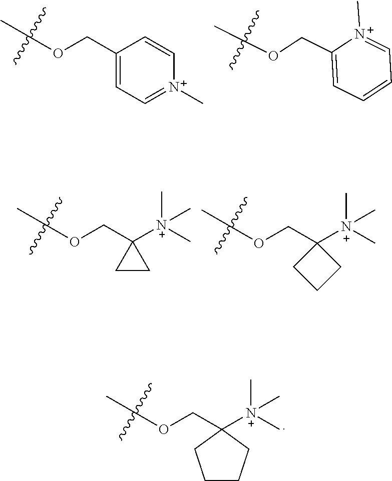

Further, R₁ is selected from hydroxyl, hydrogen or one of the following structures:

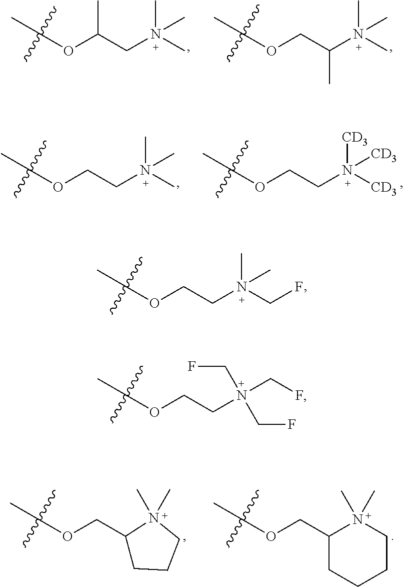

Still further, R₁ is selected from hydroxyl or one of the following structures:

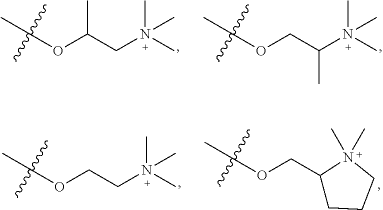

Further, R₇ is selected from C₃₋₆ lower alkyl; the lower alkyl may be a linear alkyl. Still further, R₇ is selected from n-butyl or n-pentyl.

In the present invention, the structural formula of the compound can be selected from:

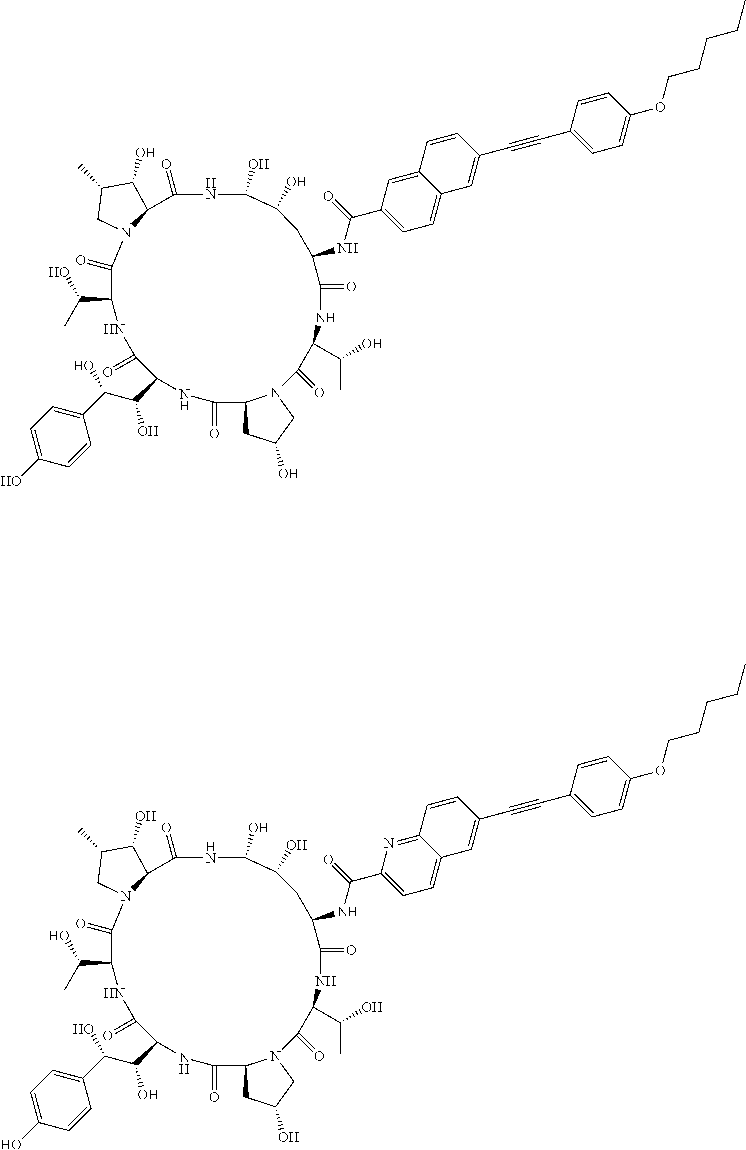 [SMILES: CCCCCOc1ccc(C#Cc2ccc3cc(C(=O)N[C@@H]4C[C@@H](O)[C@@H](O)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1 ; CCCCCOc1ccc(C#Cc2ccc3nc(C(=O)N[C@@H]4C[C@@H](O)[C@@H](O)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1]

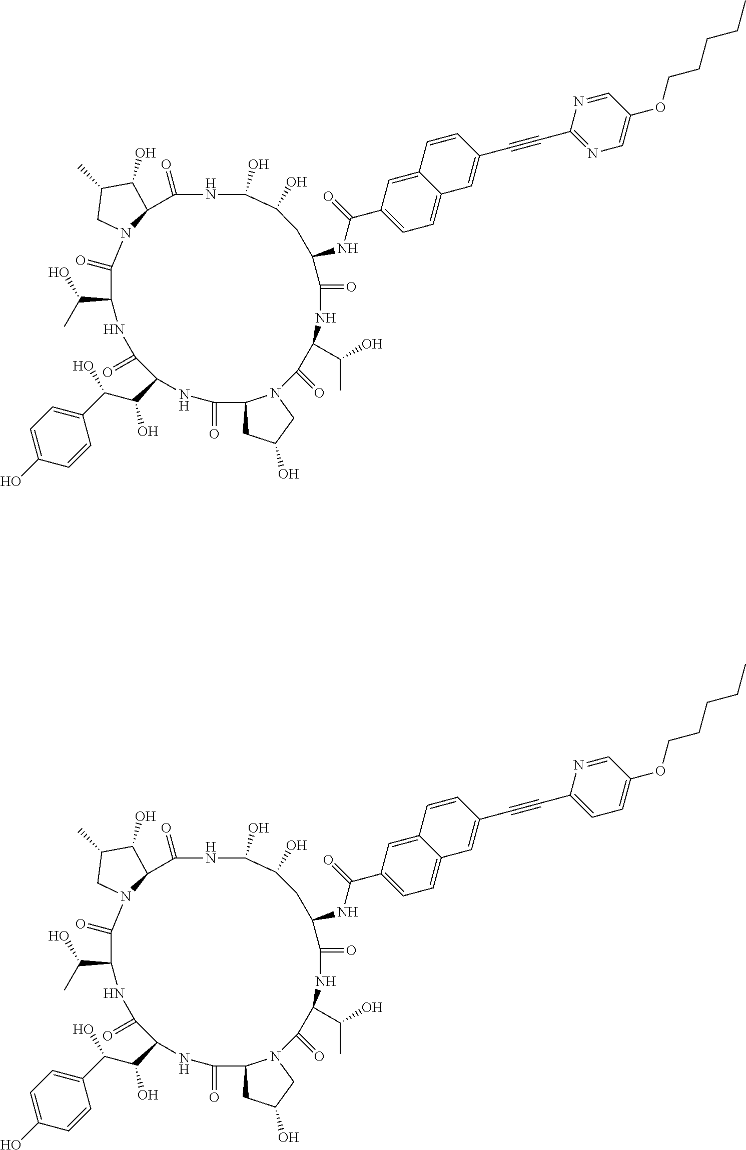 [SMILES: CCCCCOc1cnc(C#Cc2ccc3cc(C(=O)N[C@@H]4C[C@@H](O)[C@@H](O)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)nc1 ; CCCCCOc1ccc(C#Cc2ccc3cc(C(=O)N[C@@H]4C[C@@H](O)[C@@H](O)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)nc1]

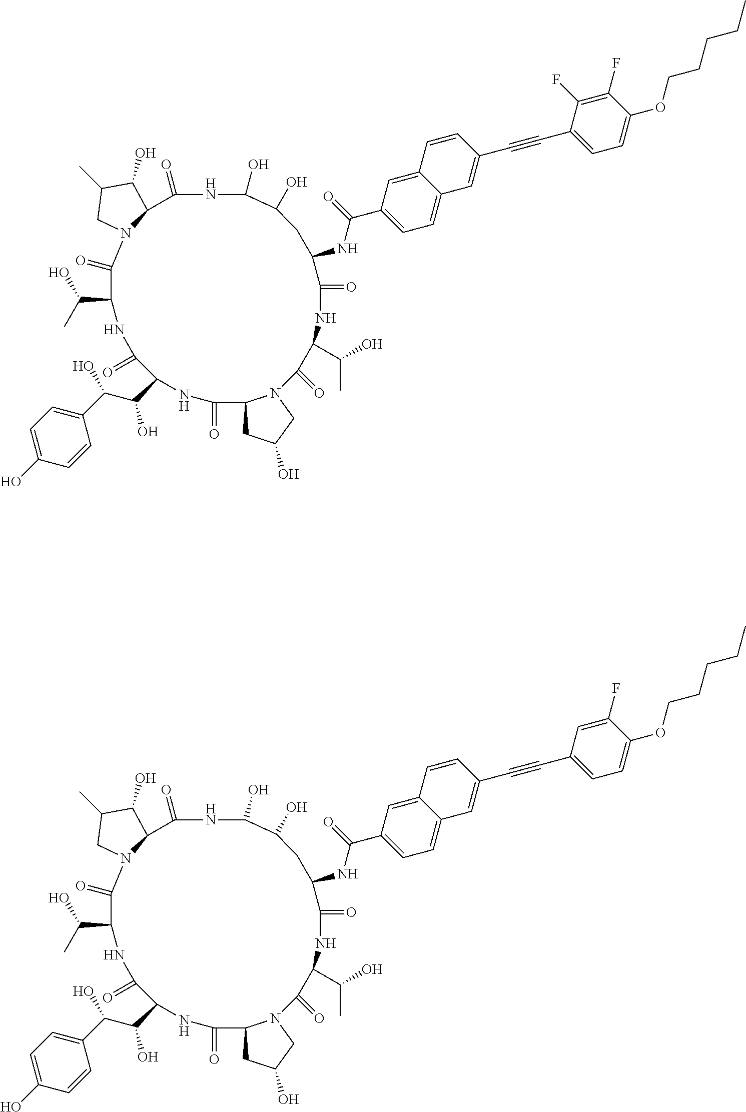

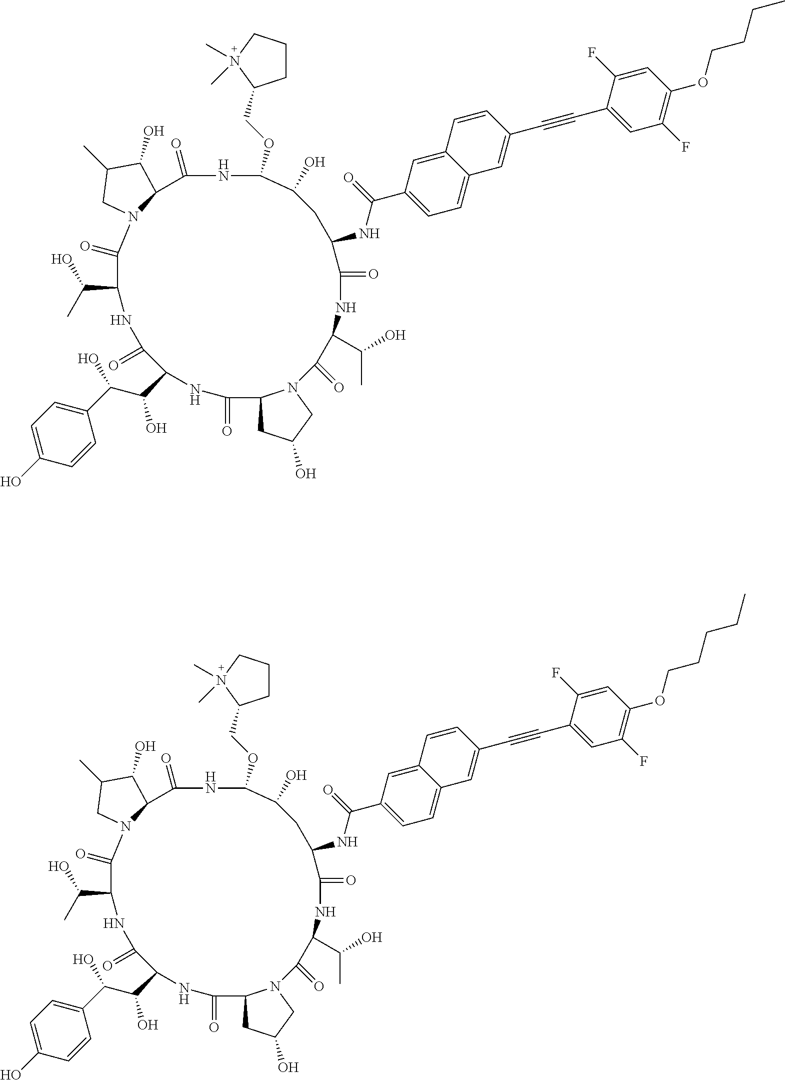

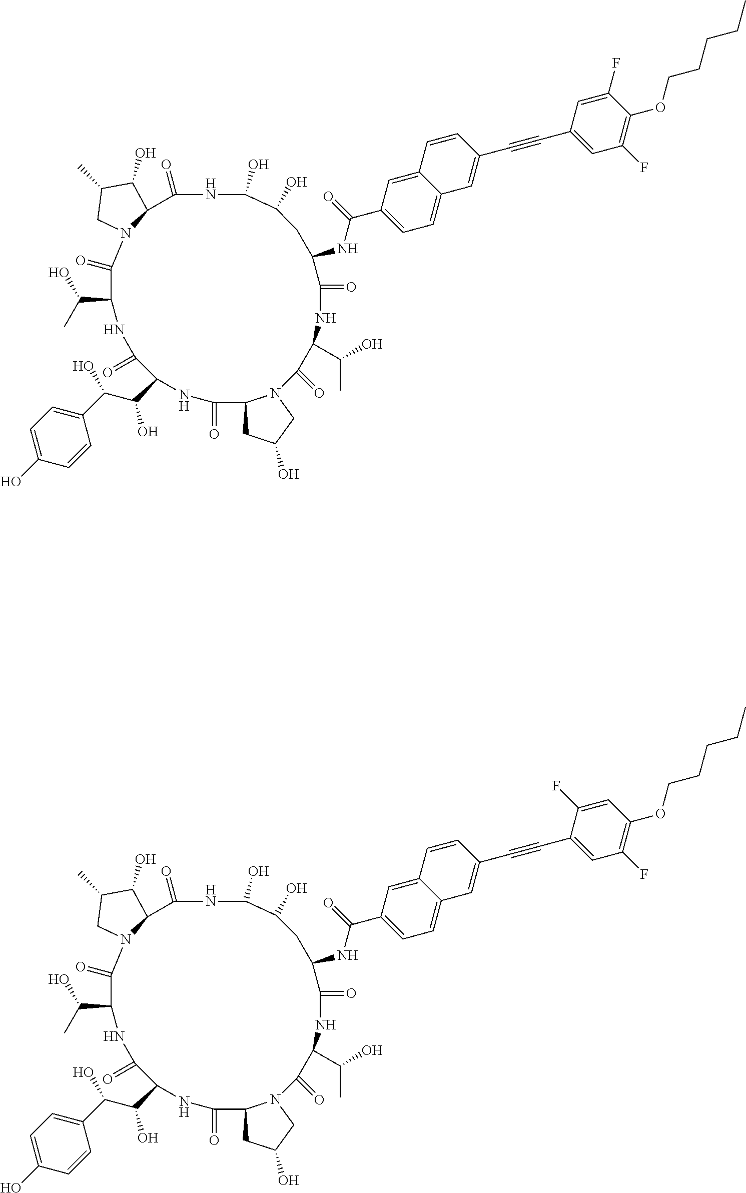

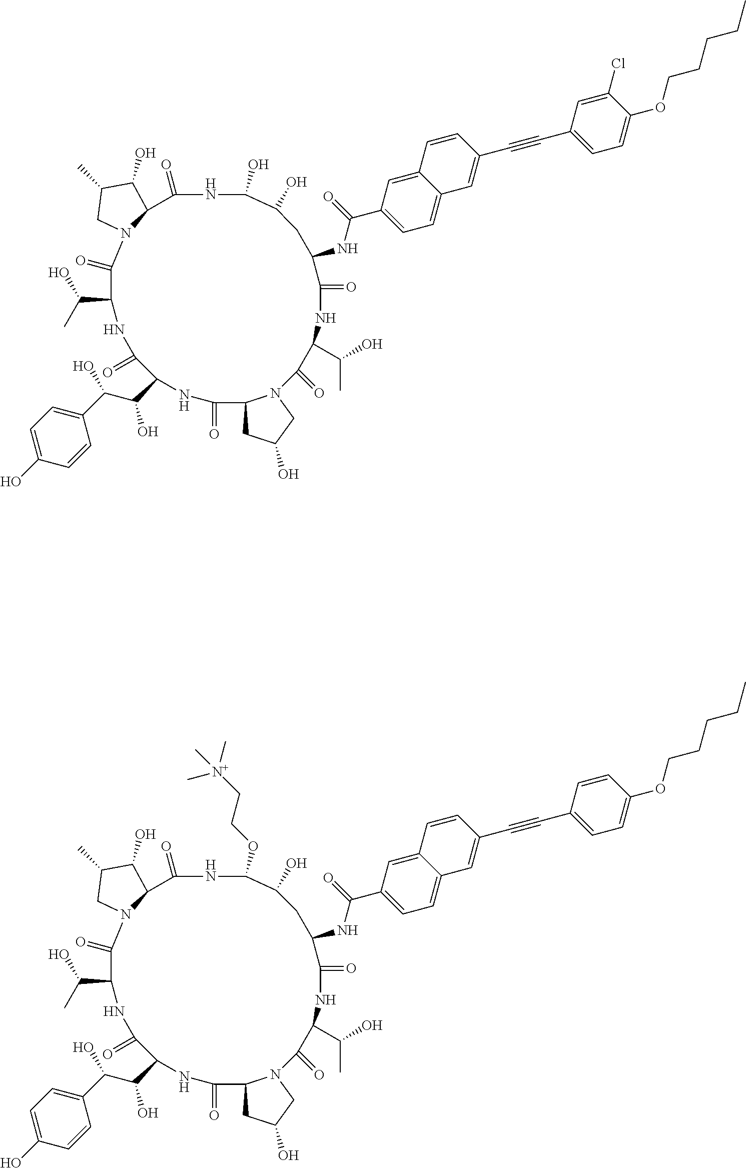

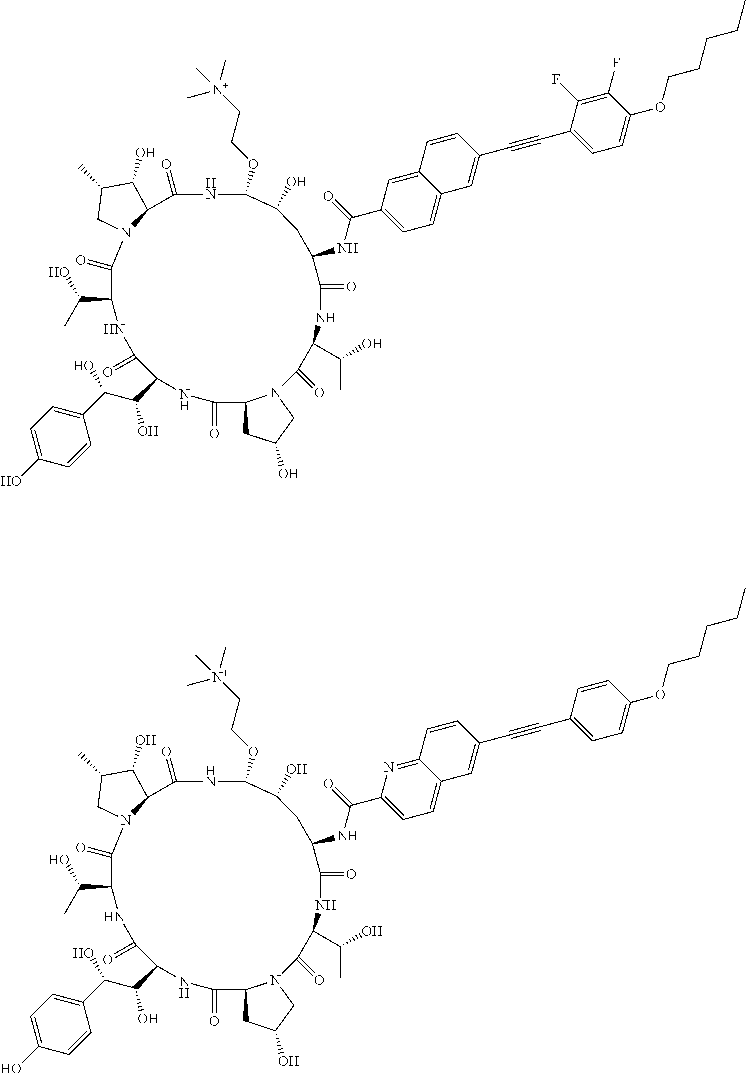 [SMILES: CCCCCOc1ccc(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)c(F)c1F ; CCCCCOc1ccc(C#Cc2ccc3nc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1]

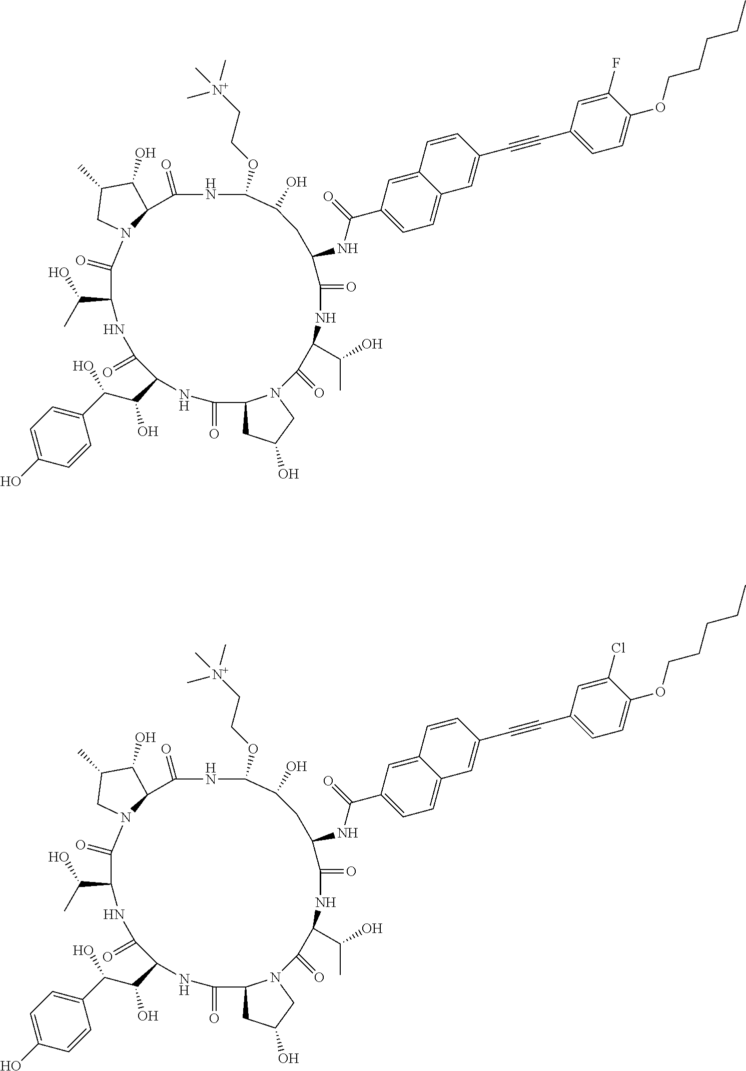

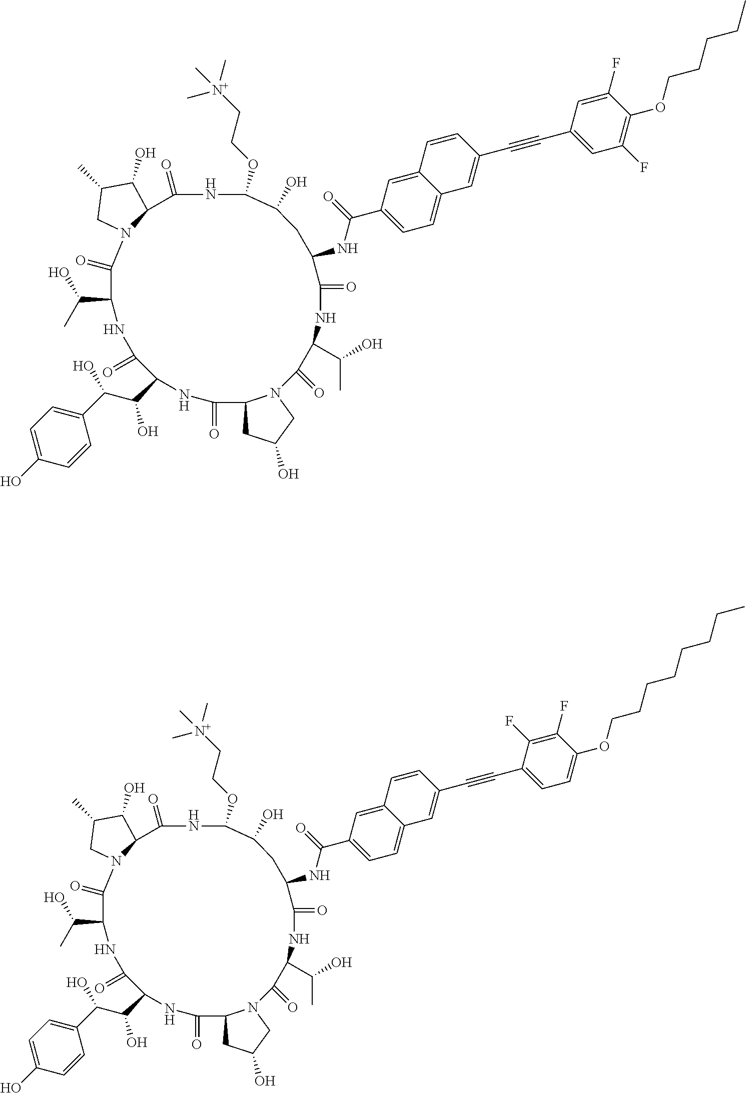 [SMILES: CCCCCOc1c(F)cc(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F ; CCCCCCCCOc1ccc(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)c(F)c1F]

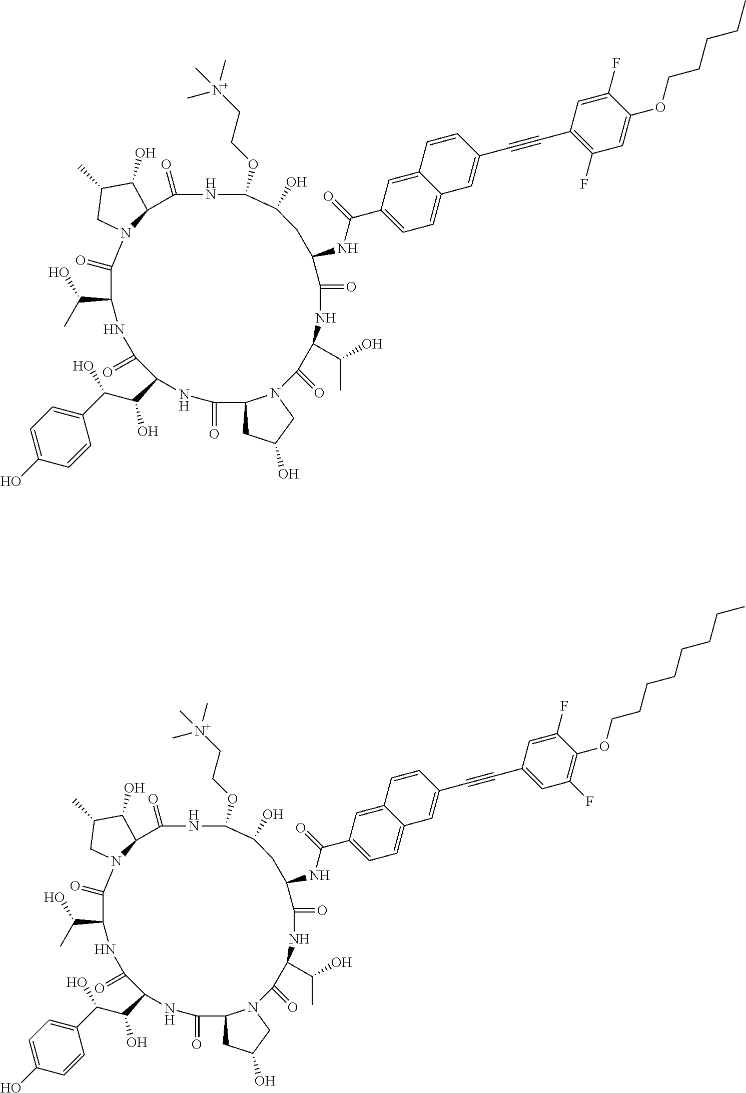 [SMILES: CCCCCOc1cc(F)c(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F ; CCCCCCCCOc1c(F)cc(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F]

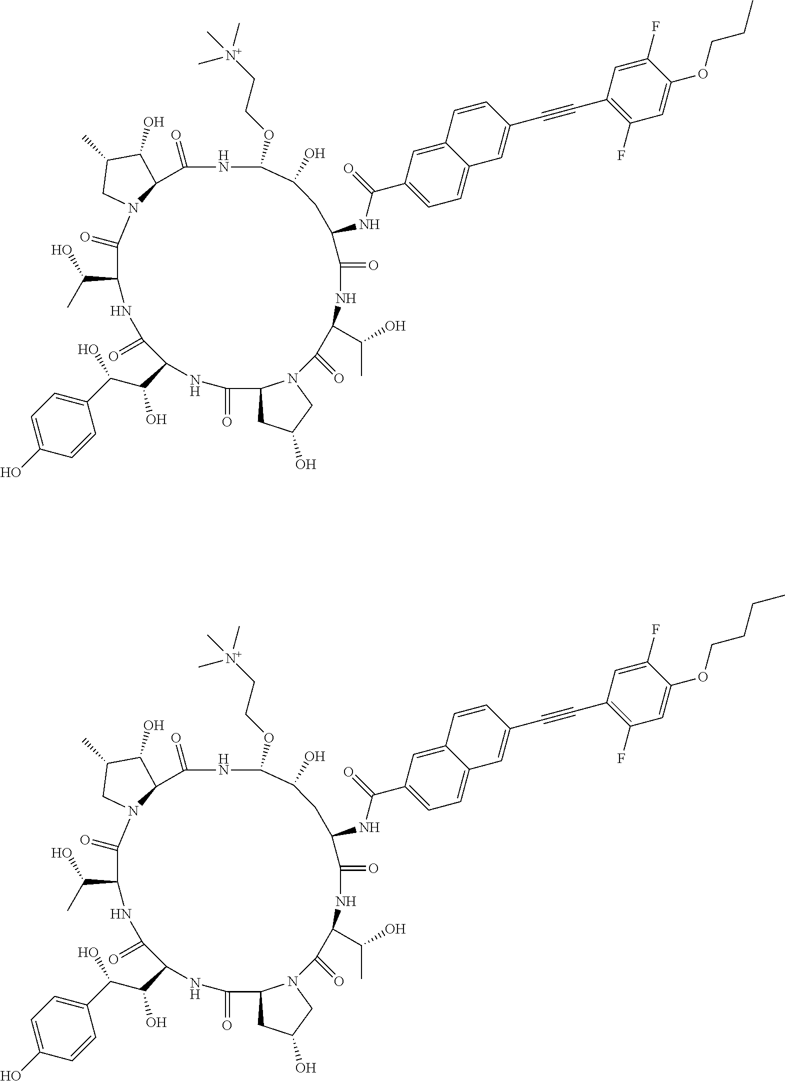 [SMILES: CCCOc1cc(F)c(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F ; CCCCOc1cc(F)c(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F]

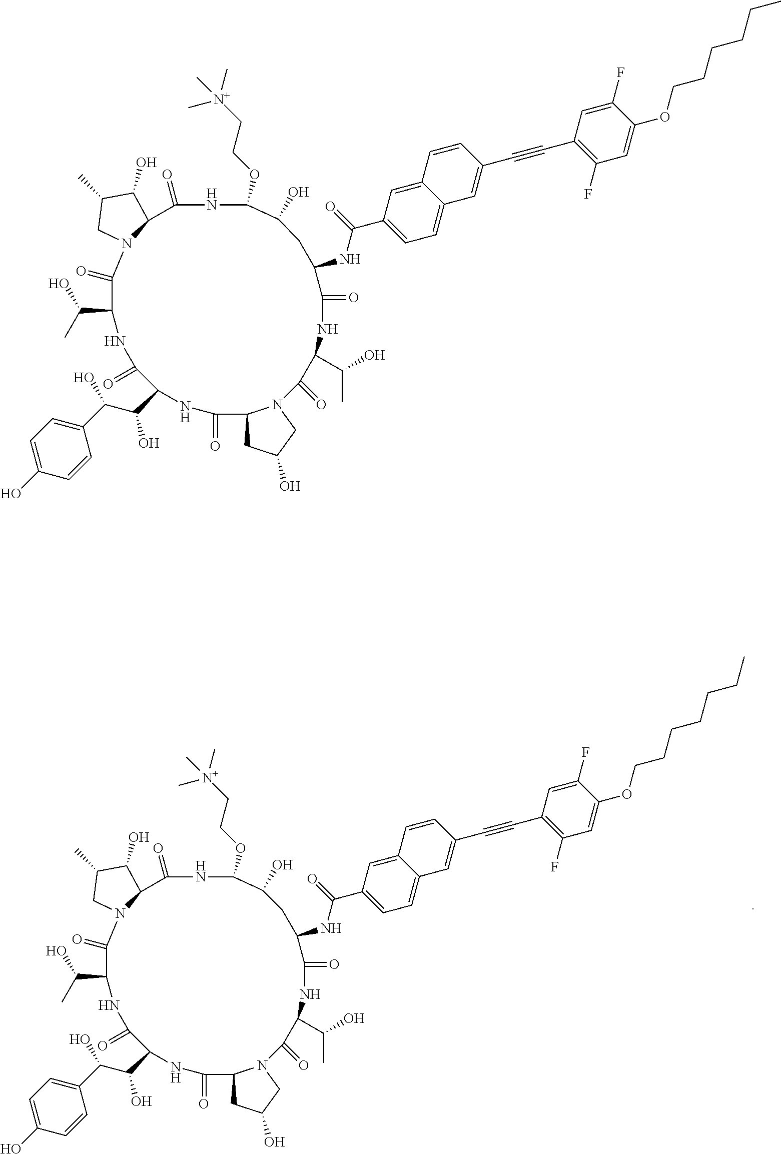 [SMILES: CCCCCCOc1cc(F)c(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F ; CCCCCCCOc1cc(F)c(C#Cc2ccc3cc(C(=O)N[C@@H]4C[C@@H](O)[C@@H](OCC[N+](C)(C)C)NC(=O)[C@@H]5[C@@H](O)[C@@H](C)CN5C(=O)[C@H]([C@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F]

The present invention further provides intermediate II of the compound of formula I:

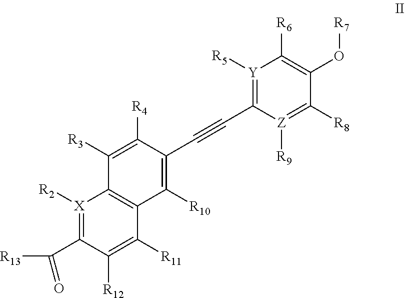

wherein the definitions of X, Y, Z, and R₂ to R₁₂ correspond to those in the compound of formula I;
and —C(═O)—R₁₃ constitutes carboxyl, acyl halide, an ester group and an anhydride group.

Further, R₁₃ is selected from —OH, Cl, —O—C(═O)CH₃, and —R_{g1};
and R_{g1} is selected from

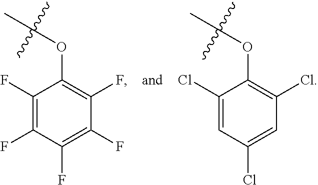 [SMILES: *Oc1c(F)c(F)c(F)c(F)c1F ; "and" ; *Oc1c(Cl)cc(Cl)cc1Cl]

The compound of formula I of the present invention can be obtained by an amidation reaction of an intermediate compound of formula II with a salt of echinocandin B; or, by a substitution reaction at the site of echinocandin B corresponding to R₁ after the amidation reaction.

The present invention further provides the use of the above-mentioned compound, or the pharmaceutically acceptable salt thereof or the isomer thereof in the preparation of a drug for treating or preventing a fungal infection or a disease caused by a fungal infection.

In the present invention, the fungus is selected from one or more organisms of the following genera: Candida albicans, C. parapsilosis, C. glabrata, C. guilliermondii, C. krusei, C. lusitaniae, C. tropicalis, Aspergillus fumigatus, A. flavus, A. terreus, A. niger, A. candidus, A. clavatus or A. ochraceus.

In the present invention, the disease caused by a fungal infection is selected from tinea capitis, tinea corporis, tinea pedis, onychomycosis, perionychomycosis, chromophytosis, thrush, vaginal candidiasis, respiratory candidiasis, biliary tract candidiasis, esophageal candidiasis, urinary tract candidiasis, systemic candidiasis, mucocutaneous candidiasis, aspergillosis, mucormycosis, paracoccidioidomycosis, North America blastomycosis, histoplasmosis, coccidioidomycosis, sporotrichosis, fungal rhino-sinusitis or chronic paranasal inflammation.

Further, the disease caused by a fungal infection is selected from candidal bacteremia and invasive candidiasis.

The present invention further provides a method for preventing a fungal infection in a patient, wherein the method is performed by administering to the patient the pharmaceutical composition of the present invention in an amount sufficient to prevent the infection. For example, the method of the present disclosure can be used for a preventive treatment in patients preparing for invasive medical interventions (such as preparing for surgery, receiving transplantation, receiving stem cell therapy, receiving graft, receiving prosthesis, receiving long-term or frequent intravenous catheterization, or receiving treatments in an intensive care unit), in immunocompromised patients (such as patients with cancers, patients with HIV/AIDS, or patients taking immunosuppressants), or in patients undergoing a long-term antibiotic therapy.

The present invention also provides a method for preventing, stabilizing, or inhibiting the growth of fungi or killing fungi, wherein the method is performed by bringing fungi or a site susceptible to fungal growth into contact with the compound of the present invention or the pharmaceutically acceptable salt thereof.

The terms “enough amount” and “sufficient amount” refer to the amount of a drug which is required for the treatment or prevention of an infection. The sufficient amount used to implement the present disclosure for the curative treatment or preventive treatment of a disorder caused by or contributed by an infection varies according to the mode of administration, the type of the infection, and the age, weight and general health of a patient.

The so-called “fungal infection” refers to the invasion of a host by a pathogenic fungus. For example, an infection may include the overgrowth of a fungus that is normally present in the body or on the body surface of a patient or the growth of a fungus that is normally not present in the body or on the body surface of a patient. More generally, a fungal infection can be any situation in which the presence of a fungal population is harmful to a host organism. Thus, when an excess fungal population is present in the body or on the body surface of a patient, or when the presence of a fungal population impairs cells or other tissues of a patient, the patient “suffers” from a fungal infection.

The term “treatment” refers to the administration of a pharmaceutical composition for the purpose of prevention and/or treatment. For the term “prevention of a disease”, it refers to the preventive treatment of a subject who does not have a disease but is susceptible to a specific disease or at risk of developing a specific disease. For the term “treatment of a disease”, it refers to the treatment of a patient who has already suffered from a disease to improve or stabilize the disorder of the patient.

The present invention further provides an antifungal pharmaceutical composition, which comprises the above-mentioned compound, or the pharmaceutically acceptable salt thereof or the isomer thereof.

The pharmaceutical composition may contain a pharmaceutically acceptable adjuvant material.

In the present invention, the term “pharmaceutically acceptable” refers to including any substance that does not interfere with the effectiveness of the biological activity of active ingredients and that is non-toxic to a host to which it is administered.

In the present invention, the “pharmaceutically acceptable adjuvant material” refers to a generic term for all additional materials in a drug other than the main drug, and the adjuvant material should have the following properties: (1) no toxic effects and no adverse effects on humans; (2) chemically stable, not susceptible to temperature, pH, storage time, etc.; (3) no compatible contraindication with the main drug, not affecting the efficacy and quality inspection of the main drug; and (4) does not interact with the packaging material. In the present invention, the adjuvant materials include, but are not limited to, fillers (diluents), lubricants (glidants or anti-adherents), dispersants, wetting agents, binders, regulators, solubilizers, antioxidants, antimicrobial agents, emulsifying agents, disintegrating agents, etc. The binders include syrup, arabic gum, gelatin, sorbitol, tragacanth gum, cellulose and derivatives thereof (such as microcrystalline cellulose, sodium carboxymethylcellulose, ethylcellulose or hydroxypropylmethylcellulose), gelatin paste, syrup, starch paste or polyvinylpyrrolidone, etc.; the fillers include lactose, powdered sugar, dextrin, starch and derivatives thereof, cellulose and derivatives thereof, inorganic calcium salts (such as calcium sulfate, calcium phosphate, calcium hydrogen phosphate, and precipitated calcium carbonate), sorbitol or glycine, etc.; the lubricants include microsilica gel, magnesium stearate, talc powder, aluminum hydroxide, boric acid, hydrogenated vegetable oil, polyethylene glycol, etc.; the disintegrating agents includes starch and derivatives thereof (such as sodium carboxyl methyl starch, sodium starch glycolate, pre-gelatinized starch, modified starch, hydroxypropyl starch, and corn starch), polyvinylpyrrolidone or microcrystalline cellulose, etc.; the wetting agents include sodium dodecyl sulfate, water or alcohol, etc.; the antioxidants include sodium sulfite, sodium bisulfite, sodium metabisulfite, dibutyl benzoic acid, etc.; the antimicrobial agents include 0.5% phenol, 0.3% cresol, 0.5% chlorobutanol, etc.; the regulators include hydrochloric acid, citric acid, potassium (sodium) hydroxide, sodium citrate and buffer agents (including sodium dihydrogen phosphate and disodium hydrogen phosphate), etc.; the emulsifying agents include polysorbate-80, sorbitan gallate, pluronic F-68, lecithin, bean phospholipid, etc.; and the solubilizers includes Tween-80, bile, glycerol, etc.

The term “pharmaceutically acceptable salt” refers to a salt that is formed by the compound of the present invention and an acid or base and is suitable for use as a drug. The above-mentioned acid or base is a Lewis acid or base in a broad sense. The acids suitable for forming a salt include, but are not limited to: inorganic acids such as hydrochloric acid, hydrobromic acid, hydrofluoric acid, sulfuric acid, nitric acid and phosphoric acid, and organic acids such as formic acid, acetic acid, propionic acid, oxalic acid, malonic acid, succinic acid, fumaric acid, maleic acid, lactic acid, malic acid, tartaric acid, citric acid, picric acid, methanesulfonic acid, benzenemethanesulfonic acid and benzenesulfonic acid; and acidic amino acids such as aspartic acid and glutamic acid.

The method of administration of the compound or the pharmaceutical composition of the present invention is not particularly limited, and representative methods of administration include (but are not limited to): oral, parenteral (intravenous, intramuscular or subcutaneous), and topical administration.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with: (a) a filler or compatibilizer, such as starch, lactose, sucrose, glucose, mannitol and silicic acid; (b) a binder, such as hydroxymethylcellulose, alginate, gelatin, polyvinylpyrrolidone, sucrose and arabic gum; (c) a humectant, such as glycerol; (d) a disintegrant, such as agar, calcium carbonate, potato starch or cassava starch, alginic acid, certain complex silicates, and sodium carbonate; (e) a retarding solvent, such as paraffin wax; (f) an absorption accelerator, such as quaternary amine compounds; (g) a wetting agent, such as cetyl alcohol and glyceryl monostearate; (h) an adsorbent, such as kaolin; and (i) a lubricant, such as talc, calcium stearate, magnesium stearate, solid polyethylene glycol, sodium dodecyl sulfate, or mixtures thereof. In capsules, tablets and pills, the dosage forms may also comprise a buffering agent.

Solid dosage forms such as tablets, sugared pills, capsules, pills and granules may be prepared using coatings and shell materials, such as enteric coatings and other materials well known in the art. They may comprise an opacifying agent, and the release of the active compound or the compound in such composition may be delayed in a certain part of the digestive tract. Examples of embedding components that can be used are polymeric substances and wax substances. If necessary, the active compound can also form a microcapsule with one or more of the above-mentioned excipients.

Liquid dosage forms for oral administration include pharmaceutically acceptable emulsions, solutions, suspensions, syrups or tinctures. In addition to the active compound, the liquid dosage forms may comprise common inert diluents in the art, such as water or other solvents, solubilizers and emulsifying agents, for example, ethanol, isopropyl alcohol, ethyl carbonate, ethyl acetate, propylene glycol, 1,3-butanediol, dimethylformamide and oils, particularly cottonseed oil, peanut oil, corn germ oil, olive oil, castor oil and sesame oil or mixtures of these substances.

In addition to these inert diluents, the composition may also comprise an adjuvant, such as a wetting agent, an emulsifying agent, a suspending agent, a sweetener, a flavoring agent and a perfume.

In addition to the active compound, the suspension may comprise a suspending agent, such as ethoxylated isostearyl alcohol, polyoxyethylene sorbitol, sorbitan ester, microcrystalline cellulose, aluminum methoxide and agar, or mixtures of these substances.

The composition for parenteral injection may comprise a physiologically acceptable sterile aqueous or anhydrous solution, dispersion, suspension or emulsion, and a sterile powder for redissolving into a sterile injectable solution or dispersion. Suitable aqueous and non-aqueous carriers, diluents, solvents or excipients include water, ethanol, polyols and suitable mixtures thereof.

The dosage forms of the compound of the present invention for topical administration include ointments, powders, patches, sprays and inhalants. The active ingredient is mixed under sterile conditions with a physiologically acceptable carrier and any preservatives, buffers, or propellants that may be required if necessary.

The compound of the present invention can also be used in an injectable preparation. Specifically, the injectable preparation is selected from a liquid injection (water needle), a sterile powder for injection (powder needle) or a tablet for injection (referring to a molded tablet or a compressed tablet prepared from a drug by means of aseptic operation, which is dissolved with water for injection immediately before use, and used for subcutaneous or intramuscular injection).

Specifically, the powder for injection comprises at least an excipient in addition to the above-mentioned compound. In the present invention, the excipients are ingredients which are intentionally added to a drug in an amount by which same is supposed to not have pharmacological properties, but the excipients can contribute to the processing, dissolving or dissolution of the drug, drug delivery through targeted drug administration routes or stability.

Functional group isomers resulting from the rapid movement of an atom in a molecule at two positions are called tautomers.

Mesomer molecules contain atoms of asymmetry, but have a factor of symmetry such that the total optical rotation within the molecule is zero, i.e., no optical rotation.

A racemate is an equimolar mixture of a chiral molecule (see chirality) with optical activity (see optical isomerism) and its enantiomer.

Stereoisomers in which the molecules are objects and mirror images of each other and are non-superimposable are called enantiomers. The enantiomers all have optical rotation, one of which is levorotatory and one of which is dextrorotatory, so the enantiomers are also known as optical isomers.

Diastereoisomers refer to stereoisomers whose molecules have two or more chiral centers and whose molecules are not mirror images of each other.

The “independently selected from” refers to variable groups at each occurrence being each independently selected from a defined substituent.

The “alkyl” refers to a linear or branched alkane group, preferably containing 1 to 10 carbon atoms, more preferably containing 3 to 7 carbon atoms. Non-limiting examples include methyl, ethyl, n-propyl, isopropyl, n-butyl, sec-butyl, tert-butyl, pentyl, hexyl, heptyl, octyl, nonyl, and decyl. Unless otherwise stated in the description, the alkyl can be optionally substituted with one or more of the following substituents: halogen, cyano, thiocyano, isothiocyano, nitro, oxo, thio, trimethylsilyl, etc.

Unless particularly stated, the “lower alkyl” refers to a branch chain or branched alkane group containing 1 to 10 carbon atoms. Non-limiting examples include methyl, ethyl, n-propyl, isopropyl, n-butyl, sec-butyl, tert-butyl, pentyl, hexyl, heptyl, octyl, nonyl, and decyl. Unless otherwise stated in the description, the lower alkyl can be optionally substituted with one or more of the following substituents: halogen, cyano, thiocyano, isothiocyano, nitro, oxo, thio, trimethylsilyl, etc.

The “alkenyl” refers to an alkyl compound containing a carbon-carbon double bond within the molecule, and the alkyl is defined as described above. Non-limiting examples include vinyl, 1-propen-2-yl, 1-buten-4-yl, 1-penten-5-yl, 1-buten-1-yl, etc. Unless otherwise stated, the alkenyl can be optionally substituted with one or more of the following substituents: halogen, cyano, thiocyano, isothiocyano, nitro, oxo, thio, trimethylsilyl, etc.

The “alkynyl” refers to an alkyl compound containing a carbon-carbon triple bond within the molecule, and the alkyl is defined as described above. Non-limiting examples include ethynyl, propynyl, butynyl, pentynyl, etc. Unless otherwise stated, the alkynyl can be optionally substituted with one or more of the following substituents: halogen, cyano, thiocyano, isothiocyano, nitro, oxo, thio, trimethylsilyl, etc.

The “aryl” refers to a hydrocarbon ring system group containing a hydrogen atom, 6 to 14 carbon atoms and at least one aromatic ring. It may be a monocyclic ring, bicyclic ring or tricyclic ring system, and it may include a spiro ring system. The aryl includes, but is not limited to, aryl derived from acenaphthene, anthracene, azulene, benzene, 6,7,8,9-tetrahydro-5H-benzo[7]annulene, fluorene, indene, naphthalene, phenalene and phenanthrene. Unless particularly stated, the aryl can be optionally substituted with one or more substituents independently selected from the following groups: alkyl, alkenyl, alkynyl, halogen, haloalkyl, haloalkenyl, haloalkynyl, cyano, nitro, etc.

The “cyclic hydrocarbonyl” refers to a stable non-aromatic monocyclic ring or polycyclic ring hydrocarbonyl consisting only of carbon atoms and hydrogen atoms, which may contain a spiro ring or bridged ring system and have 3 to 15 carbon atoms, 3 to 10 carbon atoms or 5 to 7 carbon atoms, and which is saturated or unsaturated and is linked to the rest of the molecule via a single bond. The monocyclic cyclic hydrocarbonyl includes non-bridged cyclic hydrocarbonyl, such as cyclopropyl, cyclobutyl, cyclopentyl, cyclohexyl, cycloheptyl and cyclooctyl. Polycyclic groups include a fused ring, spiro ring or bridged ring hydrocarbonyl, for example, C10 groups, such as adamantyl (bridged) and decahydronaphthyl (fused); C7 groups, such as bicyclo[3.2.0]heptyl (fused), norbornanyl and norbornenyl (bridged); and substituted polycyclic groups, such as substituted C7 groups, such as 7,7-dimethylbicyclo[2.2.1]heptyl (bridged), etc. Unless otherwise stated, the cyclic hydrocarbonyl can be optionally substituted with one or more substituents independently selected from the following groups: alkyl, alkenyl, alkynyl, halogen, haloalkyl, haloalkenyl, haloalkynyl, oxo, thio, cyano, nitro, etc.

The “cycloalkyl” refers to a saturated monocyclic or polycyclic cyclic hydrocarbon substituent, having 3 to 15 carbon atoms, 3 to 10 carbon atoms, or 5 to 7 carbon atoms. Non-limiting examples of monocyclic cycloalkyl include cyclopropyl, cyclobutyl, cyclopentyl, cyclohexyl, cycloheptyl, cyclooctyl, etc.; and non-limiting examples of polycyclic cycloalkyl include spiro ring, fused ring and bridged ring cycloalkyl.

The “halogen” refers to fluorine, chlorine, bromine or iodine.

The “heterocyclyl” refers to a stable 3-membered to 18-membered non-aromatic ring group that contains 1 to 12 carbon atoms and 1 to 6 heteroatoms selected from nitrogen, oxygen and sulfur. Unless particularly stated in the description, the heterocyclyl may be a monocyclic ring, bicyclic ring, tricyclic ring or tetracyclic ring system, which may include a spiro ring or bridged ring system; the nitrogen, carbon or sulfur atoms in the heterocyclyl can be optionally oxidized; the nitrogen atom can be optionally quaternized; and the heterocyclyl can be partially or completely saturated. Unless particularly stated in the description, the heterocyclyl includes heterocyclyl optionally substituted with one or more substituents selected from the following groups: alkyl, alkenyl, alkynyl, halogen, haloalkyl, haloalkenyl, haloalkynyl, oxo, thio, cyano, nitro, etc.

Typical heterocycloalkyl includes, but is not limited to:

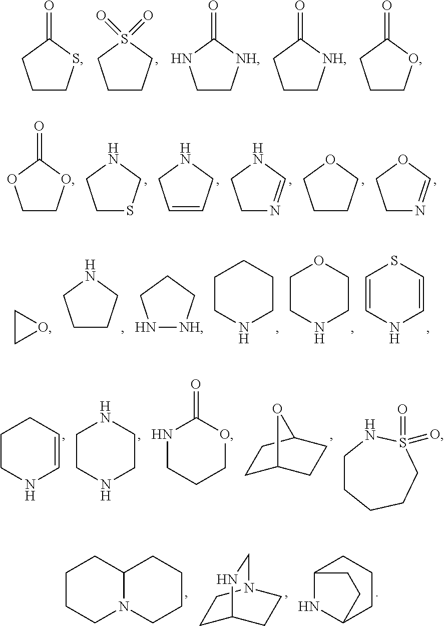

The “heteroaryl” refers to a 5-membered to 14-membered ring system group that contains a hydrogen atom, 1 to 13 carbon atoms, 1 to 6 heteroatoms selected from nitrogen, oxygen and sulfur, and at least one aromatic ring. The heteroaryl may be a monocyclic ring, bicyclic ring, tricyclic ring or tetracyclic ring system, which may include a spiro ring system; the nitrogen, carbon or sulfur atoms in the heteroaryl can be optionally oxidized; and the nitrogen atom can be optionally quaternized. The aromatic ring of the heteroaryl does not necessarily contain a heteroatom, as long as one ring of the heteroaryl contains a heteroatom. For example, 1,2,3,4-tetrahydroisoquinolin-7-yl is considered as “heteroaryl”. Unless otherwise particularly stated in the description, the heteroaryl includes heteroaryl optionally substituted with one or more substituents selected from the following groups: alkyl, alkenyl, alkynyl, halogen, haloalkyl, haloalkenyl, haloalkynyl, oxo, thio, cyano, nitro, etc.

Typical heteroaryl includes, but is not limited to:

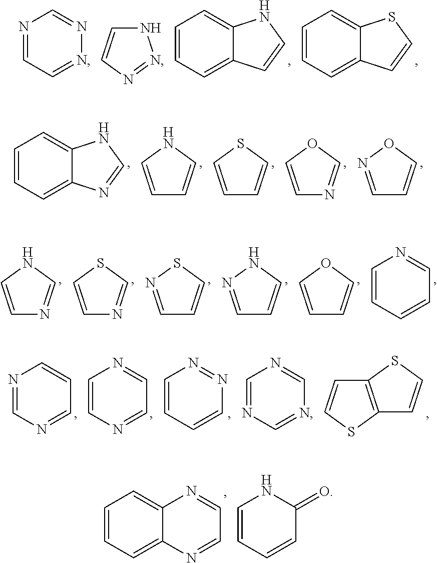

In the present invention, a compound having a novel structure is prepared by improving the structure of the lipophilic side chain linked to the cyclic hexapeptide in rezafungin. In the pharmacokinetic study, it was found that the drug exposure level (Cmax and AUC) and half-life (T_{1/2}) of this class of compounds in the blood plasma of rats were significantly superior to those of rezafungin, providing a safe and reliable new choice for clinical medication.

### BRIEF DESCRIPTION OF THE DRAWINGS

FIG. 1. Blood drug concentration-time curves after intravenous injection of rezafungin and example 16 in rats

FIG. 2. Blood drug concentration-time curves after intravenous injection of example 16 and example 19 in rats

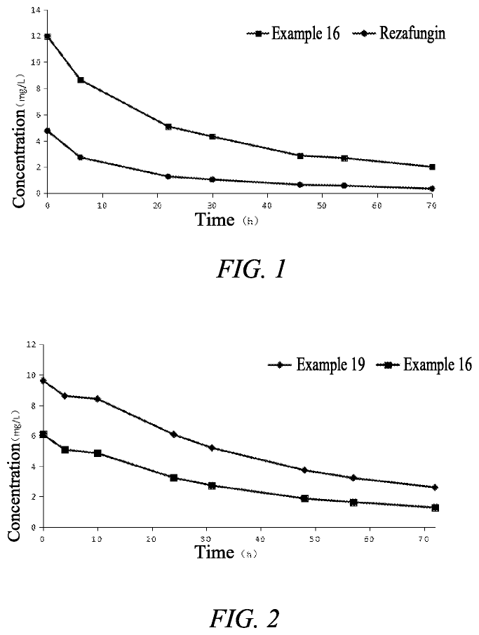

### DETAILED DESCRIPTION OF EMBODIMENTS

The technical solutions of the present invention will be described clearly and completely below. Obviously, the described embodiments are merely some of, rather than all of, the embodiments of the present invention. Based on the embodiments of the present invention, all other embodiments obtained by those of ordinary skill in the art without involving any inventive effort fall within the scope of protection of the present invention.

For the experimental methods in which no specific conditions are specified in the embodiments of the present invention, conventional conditions or conditions suggested by the raw materials or commodity manufacturers are generally used. The reagents for which the specific source is not specified, are conventional commercially available reagents.

Caspofungin was purchased from Taizhou Kede Chemical Co., Ltd.. Rezafungin was synthesized according to CN 103889221 A.

### HPLC Purity Analysis Method:

| Instrument | HPLC (SHIMADZU LC-20AT) | | | |
|---|---|---|---|---|
| Chromatographic | Xtimate Phenyl-Hexyl | | | |
| column | (4.6 mm × 150 mm, 3 μm) | | | |
| Furnace | 35° | | C. | |
| temperature | | | | |
| Mobile phase | | A: 50 mM ammonium | | |
| gradient | Time | formate + 0.1% | B: | C: |
| procedure | (min) | trifluoroacetic acid | ACN | MeOH |
| | 0 | 65 | 20 | 15 |
| | 20 | 25 | 50 | 25 |
| | 30 | 5 | 70 | 25 |
| | 35 | 5 | 70 | 25 |
| | 35.1 | 65 | 20 | 15 |
| | 45 | 65 | 20 | 15 |

| | | |
|---|---|---|
| Flow rate | 1.0 | mL/min |
| Detector | UV 254 | nm |

| | |
|---|---|
| Eluent | ACN |
| Calculation method | Area % |

### LC-MS Analysis Method:

| Instrument | UPLC-MS (Waters ACQ-ZQ) | | |
|---|---|---|---|
| Chroma- | ZORBAX SB-Aq | | |
| tographic | (2.1 mm × 30 mm, 3.5 μm) | | |
| column | | | |
| Mobile phase | | A: Aqueous | |
| gradient | Time | solution | B: |
| procedure | (min) | of 0.1% FA | Analytical ACN |
| | 0 | 65 | 35 |
| | 0.5 | 55 | 45 |
| | 1.5 | 25 | 75 |
| | 2 | 10 | 90 |
| | 2.5 | 2 | 95 |
| | 2.51 | 65 | 35 |
| | 3 | 65 | 35 |

| | | |
|---|---|---|
| Flow rate | 0.5 | mL/min |
| Furnace | 40° | C. |
| detector | | |
| Detector | UV 200 | nm |

| | |
|---|---|
| Ion source | ESI |
| Mass | 90-100 |
| scan range | |
| Mode | Positive polarity |

### Example 1

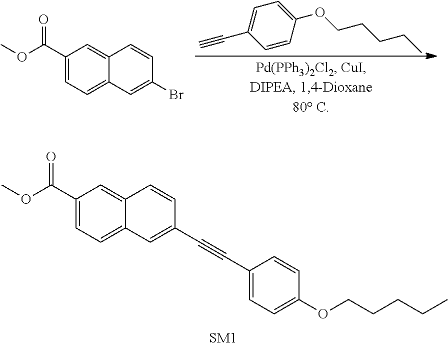 [SMILES: COC(=O)c1ccc2cc(Br)ccc2c1 ; C#Cc1ccc(OCCCCC)cc1 ; CCCCCOc1ccc(C#Cc2ccc3cc(C(=O)OC)ccc3c2)cc1]

Methyl 6-bromo-2-naphthoate (704 mg, 2.60 mmol, 1 eq.), Pd(PPh₃)₂Cl₂ (186.4 mg, 0.26 mmol, 0.1 eq.), and CuI (50.5 mg, 0.26 mmol, 0.1 eq.) were added to a thick-walled pressure-resistant reactor tube. Vacuumizing and replacement with nitrogen were performed, and repeated three times. 1-Eth-1-ynyl-4-(pentyloxy)benzene (500 mg, 2.00 mmol, 1 eq.), DIPEA (0.92 mL, 5.30 mmol, 2 eq.) and 1,4-dioxane (9 mL) were then added to the system under nitrogen atmosphere. After feeding was completed, the tube was sealed and heated to 80° C., and the mixture was stirred overnight. After the reaction was completed, the solvent was removed by using a rotary evaporator to obtain an oily substance. The oily substance was dissolved with DCM. The organic phase was washed with water and brine in sequence, and dried with anhydrous sodium sulfate. The crude product obtained after concentration was separated and purified by column chromatography (PE:EA=100:1) to obtain 403.4 mg of compound SM1 as a yellow solid, with a yield of 40%. MS[M+H]⁺: 373.

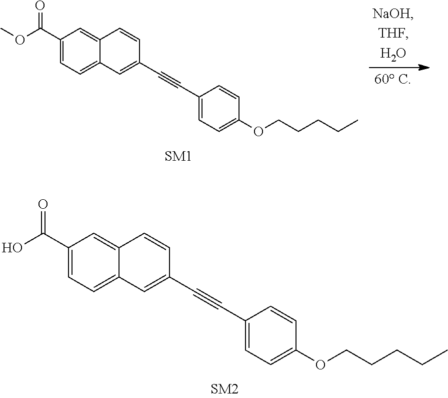 [SMILES: CCCCCOc1ccc(C#Cc2ccc3cc(C(=O)OC)ccc3c2)cc1 ; CCCCCOc1ccc(C#Cc2ccc3cc(C(=O)O)ccc3c2)cc1]

SM1 (403.4 mg, 1.08 mmol, 1 eq.) was dissolved in THF (10 mL). NaOH (86.7 mg, 2.10 mmol, 2 eq.) was dissolved in H₂O (10 mL). The aqueous solution of NaOH was added to a reaction system, and the reaction was placed in a 60° C. oil bath pan, and heated and stirred. After the reaction was completed, 2M HCl (aq.) was added to adjust the pH until an acidic state was reached. Suction filtration was performed. The filter cake was washed with water to obtain 369.7 mg of compound SM2 as a yellow solid, with a yield of 95%. MS[M−H]⁻: 357.

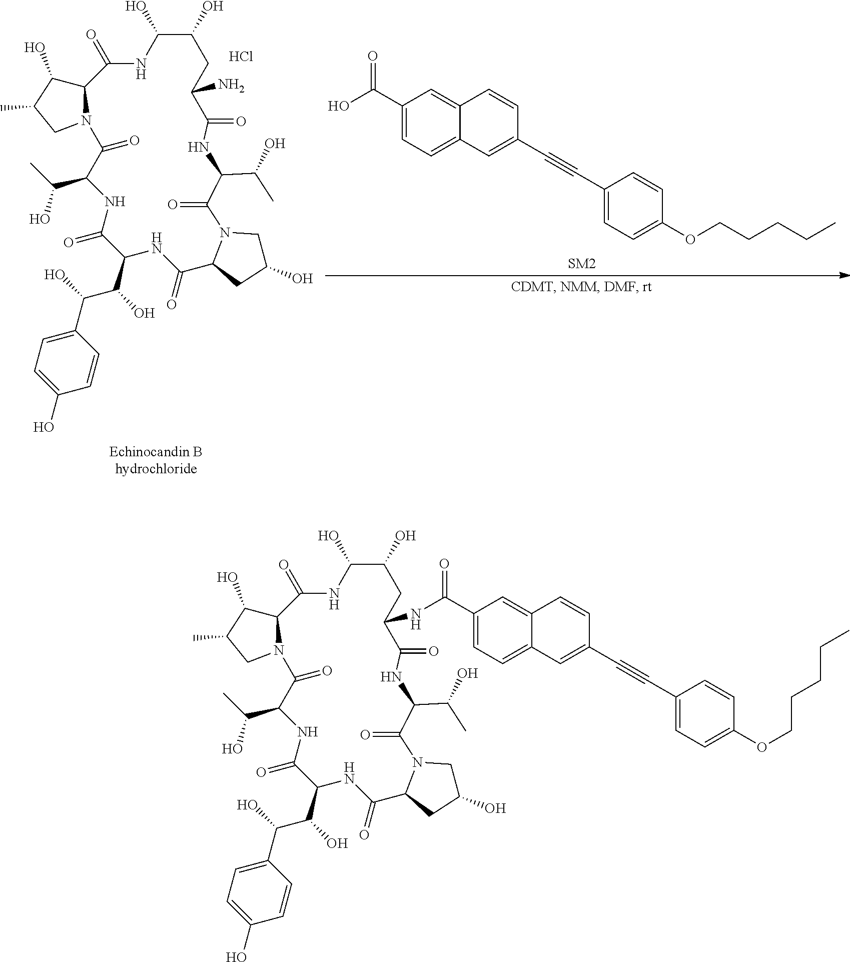 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1ccc(C#Cc2ccc3cc(C(=O)O)ccc3c2)cc1 ; CCCCCOc1ccc(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1]

Echinocandin B hydrochloride (200 mg, 0.23 mmol, 1 eq.), SM2 (85.8 mg, 0.23 mmol, 1 eq.) and CDMT (50.3 mg, 0.28 mmol, 1.2 eq.) were dissolved in DMF (2.4 mL), and then NMM (0.078 mL, 0.71 mmol, 3 eq.) was added. Stirring was performed for 1 h at room temperature. After the reaction was completed, the reaction solution was subjected to preparative HPLC (prep-HPLC) purification and separation to obtain 135 mg of a compound as a white solid, with a purity of 97% and a yield of 49%. HRMS[M−H]⁻: 1136.5260.

¹H NMR (400 MHz, CD₃OD) δ 8.41 (s, 1H), 8.05 (s, 1H), 7.93 (dd, J=15.7, 5.4 Hz, 3H), 7.61 (dd, J=8.5, 1.5 Hz, 1H), 7.49 (d, J=8.7 Hz, 2H), 7.15 (d, J=8.6 Hz, 2H), 6.95 (d, J=8.9 Hz, 2H), 6.76 (d, J=8.5 Hz, 2H), 5.36 (d, J=3.1 Hz, 1H), 5.02 (d, J=3.2 Hz, 1H), 4.70 (dd, J=11.8, 5.2 Hz, 1H), 4.57 (s, 4H), 4.35 (dd, J=19.1, 5.4 Hz, 3H), 4.26 (s, 3H), 4.08 (s, 1H), 4.02 (t, J=6.5 Hz, 3H), 3.86 (d, J=19.3 Hz, 2H), 3.41 (t, J=9.2 Hz, 1H), 2.57-2.40 (m, 2H), 2.25 (s, 1H), 2.18-2.03 (m, 2H), 1.85-1.76 (m, 2H), 1.51-1.39 (m, 4H), 1.28 (dd, J=12.5, 6.3 Hz, 6H), 1.07 (d, J=6.9 Hz, 3H), 0.96 (t, J=7.1 Hz, 3H).

### Example 2

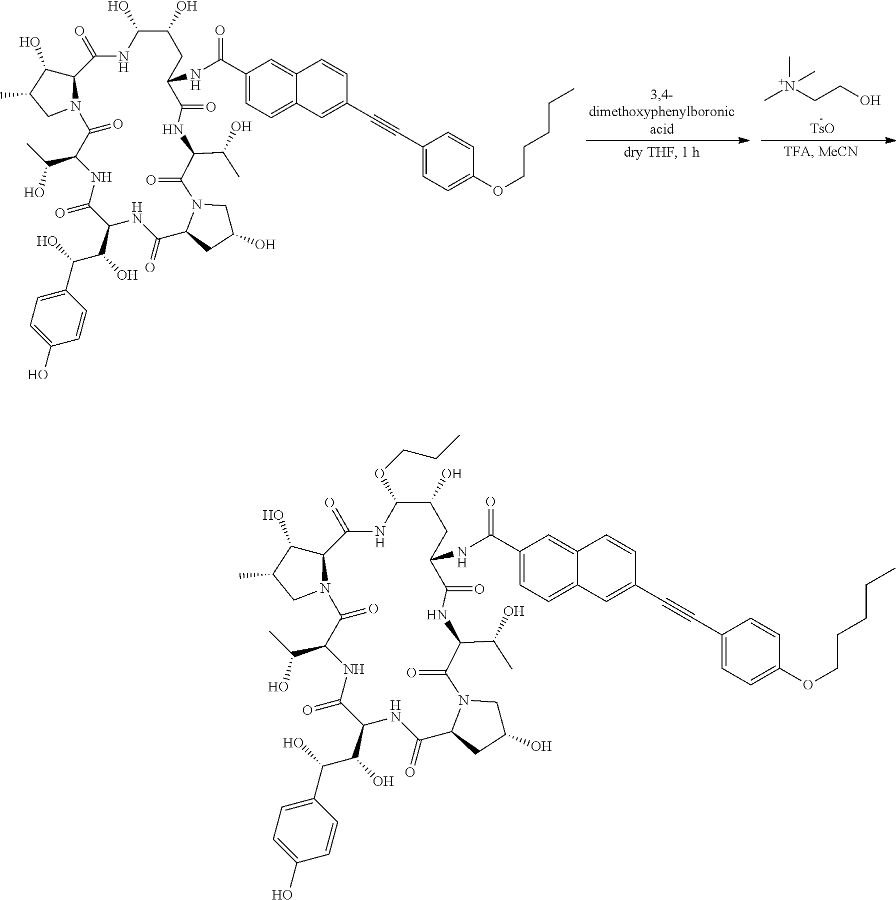

Example 1 (170 mg, 0.14 mmol, 1 eq.) and 3,4-dimethoxyphenylboronic acid (35 mg, 0.19 mmol, 1.3 eq.) were dissolved in dry THF (1.4 mL). The resulting solution was stirred for 1 h at room temperature, and concentrated to dryness. Choline tosylate (400 mg, 1.40 mmol, 10 eq.) was added, then a mixed solution of TFA (0.425 mL) and acetonitrile (1.5 mL) was added to dissolve the reactants, and stirring was performed for 6 h at room temperature under N₂ atmosphere. After the reaction was completed, an aqueous solution of sodium acetate was added for quenching, and 105 mg of a compound (hydrochloride) as a white solid was obtained through prep-HPLC purification, with a purity of 95% and a yield of 57%. HRMS[M]⁺:1223.6167.

¹H NMR (400 MHz, CD₃OD) δ 8.44 (s, 1H), 8.08 (s, 1H), 8.01-7.95 (m, 3H), 7.64 (dd, J=8.5, 1.4 Hz, 1H), 7.49 (d, J=8.7 Hz, 2H), 7.15 (d, J=8.6 Hz, 2H), 6.95 (d, J=8.9 Hz, 2H), 6.76 (d, J=8.6 Hz, 2H), 5.43 (s, 1H), 5.05 (d, J=3.2 Hz, 1H), 4.77 (d, J=5.1 Hz, 1H), 4.59 (dd, J=10.7, 7.1 Hz, 3H), 4.39 (d, J=4.2 Hz, 1H), 4.33 (d, J=8.6 Hz, 2H), 4.24 (dd, J=7.9, 1.6 Hz, 2H), 4.21-4.16 (m, 1H), 4.11 (s, 1H), 4.01 (dd, J=13.0, 6.6 Hz, 4H), 3.91 (dd, J=9.7, 7.0 Hz, 2H), 3.83 (d, J=10.8 Hz, 1H), 3.66-3.45 (m, 4H), 3.13 (s, 9H), 2.55-2.43 (m, 2H), 2.31 (dd, J=16.1, 7.2 Hz, 1H), 2.08 (dd, J=15.4, 9.5 Hz, 2H), 1.84-1.77 (m, 2H), 1.46 (ddd, J=20.2, 11.3, 6.5 Hz, 4H), 1.27 (d, J=6.3 Hz, 6H), 1.08 (d, J=6.9 Hz, 3H), 0.97 (t, J=7.1 Hz, 3H).

### Example 3

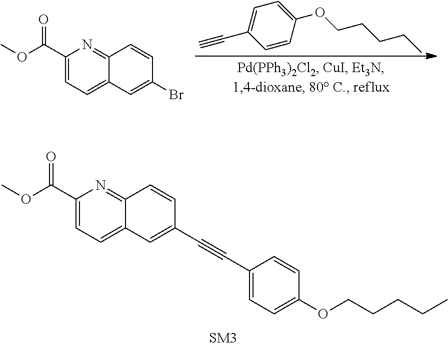 [SMILES: COC(=O)c1ccc2cc(Br)ccc2n1 ; C#Cc1ccc(OCCCCC)cc1 ; CCCCCOc1ccc(C#Cc2ccc3nc(C(=O)OC)ccc3c2)cc1]

Methyl 6-bromoquinoline-2-carboxylate (500 mg, 1.80 mmol, 1 eq.), 1-eth-1-ynyl-4-(pentyloxy)benzene (0.36 mL, 1.80 mmol, 1 eq.) and CuI (35.7 mg, 0.18 mmol, 0.1 eq.) were dissolved in 1,4-dioxane (18 mL), and then triethylamine (0.78 mL, 5.60 mmol, 3 eq.) was added. Vacuumizing and replacement with nitrogen were performed and repeated three times. Pd(PPh₃)₂Cl₂ (131 mg, 0.18 mmol, 0.1 eq.) was added to the reaction system under nitrogen atmosphere. Refluxing was performed overnight at 80° C. After the reaction was completed, the solvent was removed, and the crude product was purified by column chromatography to obtain 200 mg of compound SM3 as a white solid, with a yield of 28%. MS[M+H]⁺: 374.2.

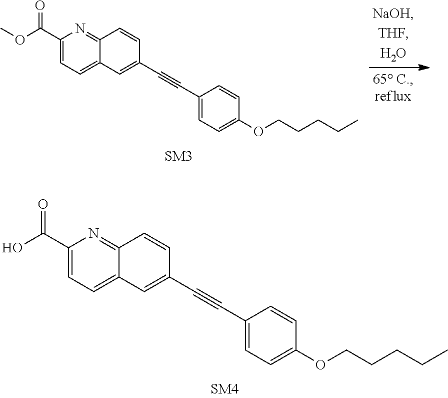 [SMILES: CCCCCOc1ccc(C#Cc2ccc3nc(C(=O)OC)ccc3c2)cc1 ; CCCCCOc1ccc(C#Cc2ccc3nc(C(=O)O)ccc3c2)cc1]

SM3 (200 mg, 0.53 mmol, 1 eq.) was dissolved in THF (5 mL), and heated and stirred at 65° C., and an aqueous solution (0.5 mL) of NaOH (43 mg, 1.08 mmol, 4 eq.) was added to the reaction system. After 1 h, the reaction solution changed from clear to turbid. TLC showed that the reaction was finished. 2M HCl (aq.) was added to adjust pH value until an acidic state is reached. Suction filtration was performed to obtain 160 mg of compound SM4 as a green solid, with a yield of 83%. MS[M+H]⁺: 360.

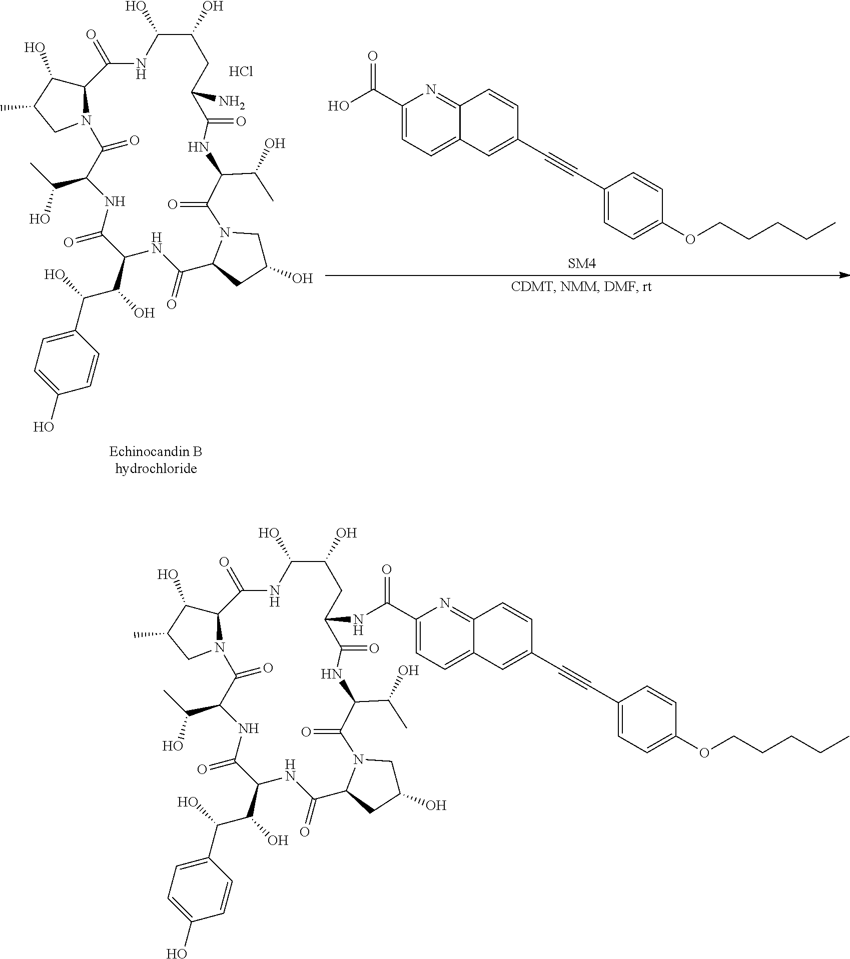 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1ccc(C#Cc2ccc3nc(C(=O)O)ccc3c2)cc1 ; CCCCCOc1ccc(C#Cc2ccc3nc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1]

Echinocandin B hydrochloride (371 mg, 0.44 mmol, 1 eq.), SM4 (160 mg, 0.44 mmol, 1 eq.) and CDMT (93.6 mg, 0.53 mmol, 1.2 eq.) were dissolved in DMF (4 mL), and then NMM (0.14 mL, 1.30 mmol, 3 eq.) was added. Stirring was performed for 1 h at room temperature. After the reaction was completed, the reaction solution was subjected to prep-HPLC purification and separation to obtain 268 mg of a compound as a white solid, with a purity of 97% and a yield of 52%.

HRMS[M−H]⁻:1137.6155.

¹H NMR (400 MHz, CD₃OD) δ 8.44 (d, J=8.6 Hz, 1H), 8.21-8.11 (m, 3H), 7.88 (dd, J=8.8, 1.7 Hz, 1H), 7.51 (d, J=8.7 Hz, 2H), 7.15 (d, J=8.5 Hz, 2H), 6.96 (d, J=8.8 Hz, 2H), 6.76 (d, J=8.5 Hz, 2H), 5.46 (d, J=2.7 Hz, 1H), 5.04 (d, J=2.8 Hz, 1H), 4.76 (dd, J=11.9, 4.8 Hz, 1H), 4.64-4.54 (m, 4H), 4.37 (d, J=2.7 Hz, 1H), 4.32 (d, J=7.6 Hz, 2H), 4.26-4.17 (m, 3H), 4.05-3.96 (m, 4H), 3.92-3.80 (m, 2H), 3.41 (t, J=9.1 Hz, 1H), 2.59-2.40 (m, 2H), 2.34 (d, J=13.6 Hz, 1H), 2.18-2.04 (m, 2H), 1.80 (dd, J=13.8, 7.2 Hz, 2H), 1.53-1.38 (m, 4H), 1.29-1.23 (m, 6H), 1.07 (d, J=6.8 Hz, 3H), 0.96 (t, J=7.1 Hz, 3H).

### Example 4

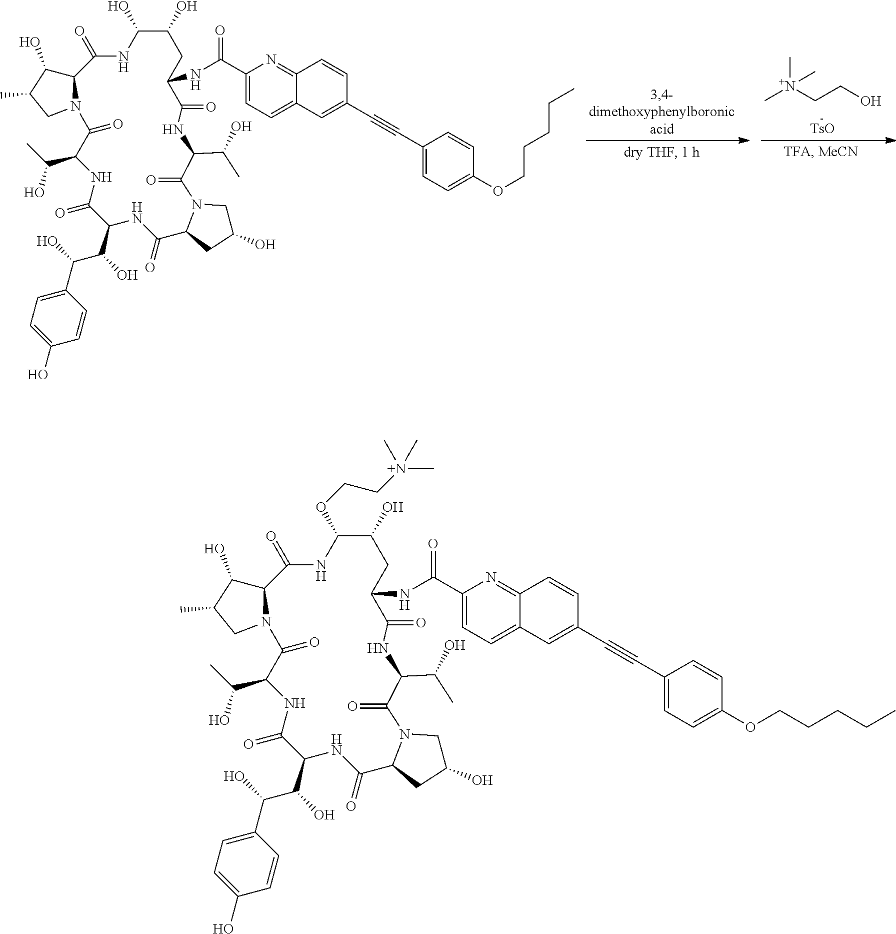

Example 3 (268 mg, 0.23 mmol, 1 eq.) and 3,4-dimethoxyphenylboronic acid (55.7 mg, 0.30 mmol, 1.3 eq.) were dissolved in dry THF (2.3 mL). The resulting solution was stirred for 1 h at room temperature, and concentrated to dryness. Choline tosylate (1.94 g, 7.00 mmol, 30 eq.) was added, then a mixed solution of TFA (0.5 mL) and acetonitrile (2.0 mL) was added to dissolve the reactants, and stirring was performed for 6 h at room temperature under N₂ atmosphere. After the reaction was completed, an aqueous solution of sodium acetate was added for quenching, and 151 mg of a compound (acetate) as a white solid was obtained through prep-HPLC purification, with a purity of 91% and a yield of 52%. HRMS[M]⁺:1224.5755.

¹H NMR (400 MHz, CD₃OD) δ 8.48 (d, J=8.6 Hz, 1H), 8.19 (dd, J=18.6, 8.8 Hz, 3H), 7.91 (d, J=10.4 Hz, 1H), 7.51 (d, J=8.7 Hz, 2H), 7.15 (d, J=8.6 Hz, 2H), 6.96 (d, J=8.7 Hz, 2H), 6.76 (d, J=8.5 Hz, 2H), 5.64 (s, 1H), 5.08 (s, 1H), 4.84-4.75 (m, 2H), 4.60 (dd, J=13.7, 4.1 Hz, 3H), 4.40 (d, J=4.1 Hz, 1H), 4.33 (t, J=8.6 Hz, 2H), 4.29-4.21 (m, 2H), 4.20-4.12 (m, 1H), 4.10-3.97 (m, 6H), 3.93-3.82 (m, 2H), 3.67-3.55 (m, 2H), 3.50-3.46 (m, 1H), 3.16 (s, 9H), 2.49 (ddd, J=22.3, 9.7, 4.8 Hz, 2H), 2.42-2.31 (m, 1H), 2.11-1.99 (m, 2H), 1.90 (s, 3H), 1.86-1.77 (m, 2H), 1.50-1.39 (m, 4H), 1.24 (dd, J=8.8, 6.4 Hz, 6H), 1.08 (d, J=6.8 Hz, 3H), 0.97 (t, J=7.1 Hz, 3H).

### Example 5

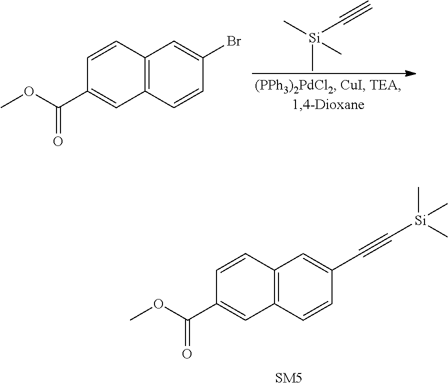

Methyl 6-bromo-2-naphthoate (4 g, 15.08 mmol, 1 eq.) was dissolved in dioxane (15 mL), and replacement with nitrogen was performed. Under the protection of nitrogen, trimethylsilylacetylene (2.08 mL, 15.08 mmol, 1 eq.), Pd(PPh₃)₂Cl₂ (1.05 g, 1.51 mmol, 0.1 eq.) and CuI (288 mg, 1.50 mmol, 0.1 eq.) were sequentially added, and triethylamine (6.29 mL, 45.26 mmol, 3 eq.) was finally added. The mixture was stirred for 2.5 h at room temperature, and extracted with water and EA. The EA phase was subjected to rotary evaporation, and separated and purified by column chromatography to obtain 4.14 g of compound SM5 as a yellow solid, with a yield of 97%. MS[M+H]⁺: 283.0.

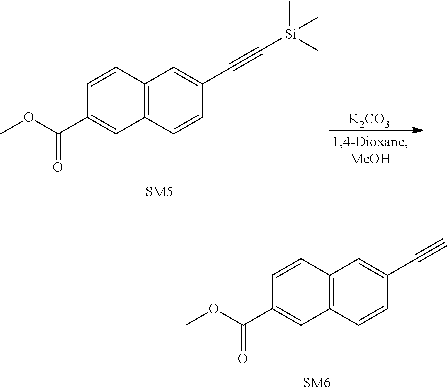

SM2 (1 g, 3.54 mmol, 1 eq.) was dissolved in dioxane (6 mL) and MeOH (6 mL). Potassium carbonate (735 mg, 5.31 mmol, 1.5 eq.) was added. The mixture was stirred for 2 h at room temperature. TLC showed that the reaction was finished. After suction filtration, the filtrate was subjected to rotary evaporation to obtain 413 mg of compound SM6 as a yellow solid, with a yield of 55%.

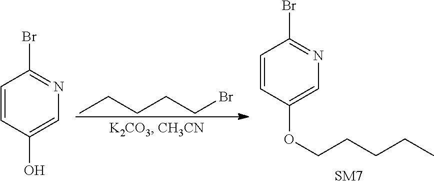

2-Bromo-5-hydroxypyridine (1 g, 5.70 mmol, 1 eq.), bromopentane (0.9 ml, 6.80 mmol, 1.2 eq.) and potassium carbonate (2.4 g, 17.00 mmol, 3 eq.) were dissolved in acetonitrile (50 mL), and the mixture was heated, refluxed, and stirred at 90° C. After 2 h, TLC showed the formation of a new spot and the disappearance of the raw materials. The solvent was removed, ethyl acetate and water were added for extraction, the organic phases were combined, dried with anhydrous sodium sulfate, and concentrated to obtain 1.3 g of compound SM7 as a green oil, with a yield of 92%.

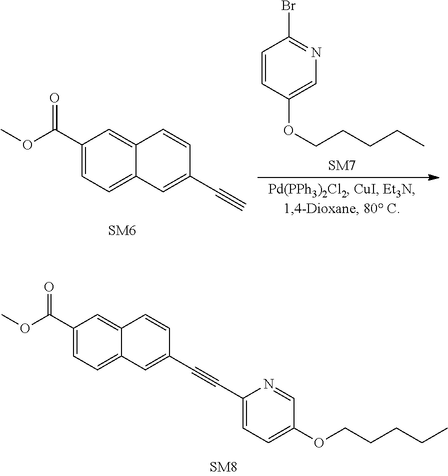 [SMILES: C#Cc1ccc2cc(C(=O)OC)ccc2c1 ; CCCCCOc1ccc(Br)nc1 ; CCCCCOc1ccc(C#Cc2ccc3cc(C(=O)OC)ccc3c2)nc1]

SM6 (156.4 mg, 0.74 mmol, 1 eq.), SM7 (200 mg, 0.81 mmol, 1.1 eq.) and CuI (14 mg, 0.074 mmol, 0.1 eq.) were dissolved in 1,4-dioxane (7 mL), and then triethylamine (0.3 mL, 2.20 mmol, 3 eq.) was added. Vacuumizing and replacement with nitrogen were performed and repeated three times. Pd(PPh₃)₂Cl₂ (52 mg, 0.07 mmol, 0.1 eq.) was added to the reaction system under nitrogen atmosphere. The resulting mixture was heated and stirred overnight at 80° C. After the reaction was completed, the solvent was removed. DCM was added for dissolving, and column chromatography purification was performed to obtain 167 mg of compound SM8 as a yellow solid, with a yield of 60%.

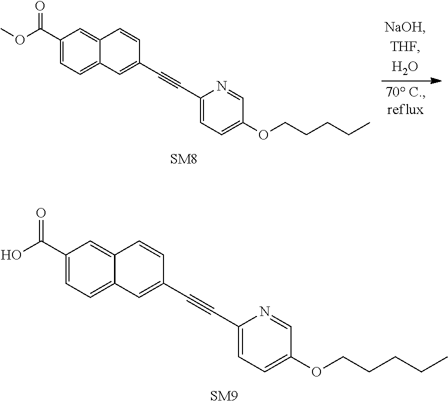 [SMILES: CCCCCOc1ccc(C#Cc2ccc3cc(C(=O)OC)ccc3c2)nc1 ; CCCCCOc1ccc(C#Cc2ccc3cc(C(=O)O)ccc3c2)nc1]

SM8 (167 mg, 0.44 mmol, 1 eq.) was dissolved in THF (4 mL), and heated and stirred at 60° C., and an aqueous solution (1 mL) of NaOH (35.8 mg, 0.89 mmol, 2 eq.) was added to the reaction system. The reaction system was heated at 70° C. and refluxed overnight. After the reaction was completed, 2M HCl (aq.) was added to adjust the pH until an acidic state is reached, and then a solid was precipitated. Suction filtration was performed to obtain 100 mg of compound SM9 as a yellow solid, with a yield of 62%. MS[M−H]⁻: 358.

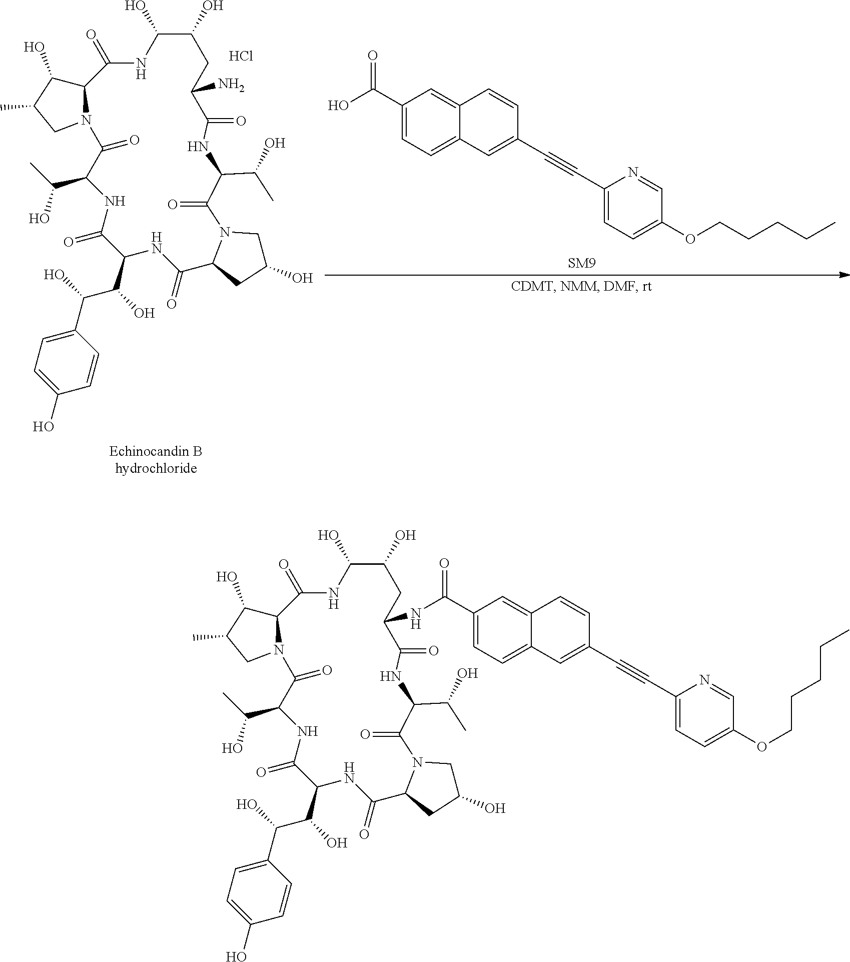 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1ccc(C#Cc2ccc3cc(C(=O)O)ccc3c2)nc1 ; CCCCCOc1ccc(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)nc1]

Echinocandin B hydrochloride (93 mg, 0.11 mmol, 1 eq.), SM9 (40 mg, 0.23 mmol, 1 eq.) and CDMT (23 mg, 0.13 mmol, 1.2 eq.) were dissolved in DMF (1.1 mL), and then NMM (0.036 mL, 0.33 mmol, 3 eq.) was added. Stirring was performed for 1 h at room temperature. After the reaction was completed, the reaction solution was subjected to prep-HPLC purification and separation to obtain 67 mg of a compound as a white solid, with a purity of 96% and a yield of 52%. HRMS[M−H]⁻: 1137.4491.

¹H NMR (400 MHz, CD₃OD) δ 8.46 (s, 2H), 8.26 (s, 1H), 8.05 (d, J=8.6 Hz, 1H), 7.98 (t, J=6.4 Hz, 3H), 7.91 (dd, J=8.9, 2.8 Hz, 1H), 7.74-7.70 (m, 1H), 7.15 (d, J=8.5 Hz, 2H), 6.76 (d, J=8.6 Hz, 2H), 5.36 (d, J=3.0 Hz, 1H), 5.02 (d, J=3.2 Hz, 2H), 4.71 (dd, J=12.9, 6.3 Hz, 1H), 4.65-4.53 (m, 3H), 4.39 (dd, J=15.0, 3.0 Hz, 1H), 4.32 (d, J=8.0 Hz, 2H), 4.28-4.19 (m, 5H), 4.14-4.06 (m, 1H), 3.99 (d, J=7.8 Hz, 1H), 3.93-3.86 (m, 1H), 3.82 (d, J=10.8 Hz, 1H), 3.44-3.38 (m, 1H), 2.59-2.41 (m, 2H), 2.31-2.21 (m, 1H), 2.10 (d, J=12.7 Hz, 2H), 1.93-1.84 (m, 2H), 1.56-1.39 (m, 4H), 1.28 (dd, J=12.4, 6.3 Hz, 6H), 1.07 (dd, J=6.8, 4.1 Hz, 3H), 0.97 (t, J=7.2 Hz, 3H).

### Example 6

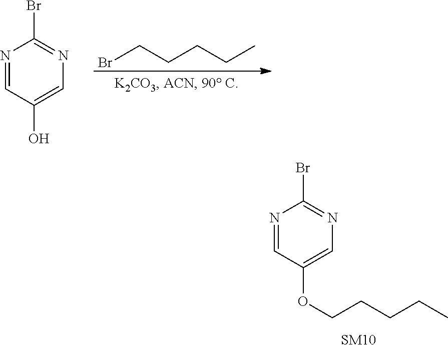

2-Bromo-5-pyrimidinol (1 g, 5.71 mmol, 1 eq.) was dissolved in acetonitrile (30 mL), bromopentane (2.1 mL, 17.14 mmol, 3 eq.) was added, and potassium carbonate (2.370 g, 17.14 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 1.24 g of compound SM10 as a brown oil, with a yield of 89%.

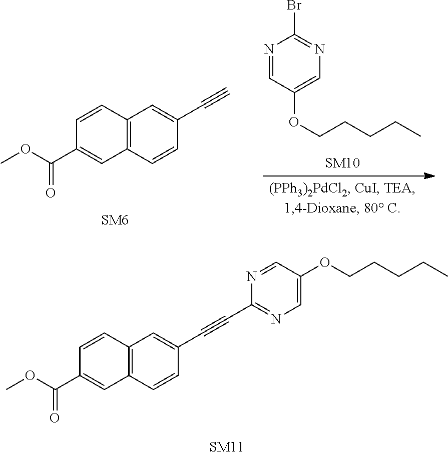 [SMILES: C#Cc1ccc2cc(C(=O)OC)ccc2c1 ; CCCCCOc1cnc(Br)nc1 ; CCCCCOc1cnc(C#Cc2ccc3cc(C(=O)OC)ccc3c2)nc1]

SM6 (500 mg, 2.04 mmol, 1 eq.) was dissolved in dioxane (8 mL), and replacement with nitrogen was performed. Under the protection of nitrogen, SM10 (428 mg, 2.04 mmol, 1 eq.), Pd(PPh₃)₂Cl₂ (143 mg, 0.20 mmol, 0.1 eq.), CuI (155 mg, 0.81 mmol, 0.4 eq.) and triethylamine (0.85 mL, 6.12 mmol, 3 eq.) were sequentially added. The mixture was stirred at 80° C. for 6 h. The solvent was removed. Extraction was performed with water and EA. The EA phase was subjected to rotary evaporation, and separated and purified by column chromatography to obtain 287 mg of compound SM11 as a yellow solid, with a yield of 38%. MS[M+H]⁺: 375.0.

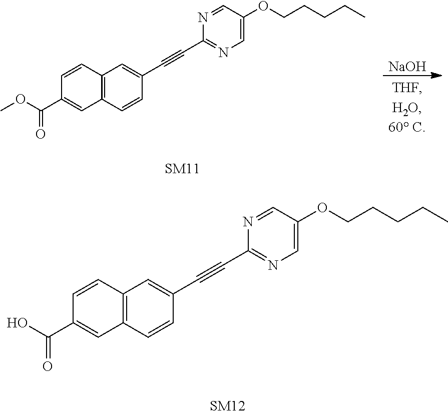 [SMILES: CCCCCOc1cnc(C#Cc2ccc3cc(C(=O)OC)ccc3c2)nc1 ; "NaOH" ; CCCCCOc1cnc(C#Cc2ccc3cc(C(=O)O)ccc3c2)nc1]

SM11 (287 mg, 0.76 mmol, 1 eq.) was dissolved in THF (7 mL). Sodium hydroxide (186 mg, 4.60 mmol, 6 eq.) was dissolved in water (1 mL) and added to the system. The system was stirred at 60° C. for 5 h, and the solvent was removed. The system was extracted with water and DCM for the first time. Dilute hydrochloric acid was added to the aqueous phase to adjust the pH until an acidic state is reached. The aqueous phase was extracted with DCM, and the solvent was removed to obtain 270 mg of crude product SM12 as a yellow-brown liquid, with a yield of 97%. MS[M+H]⁺: 361.0.

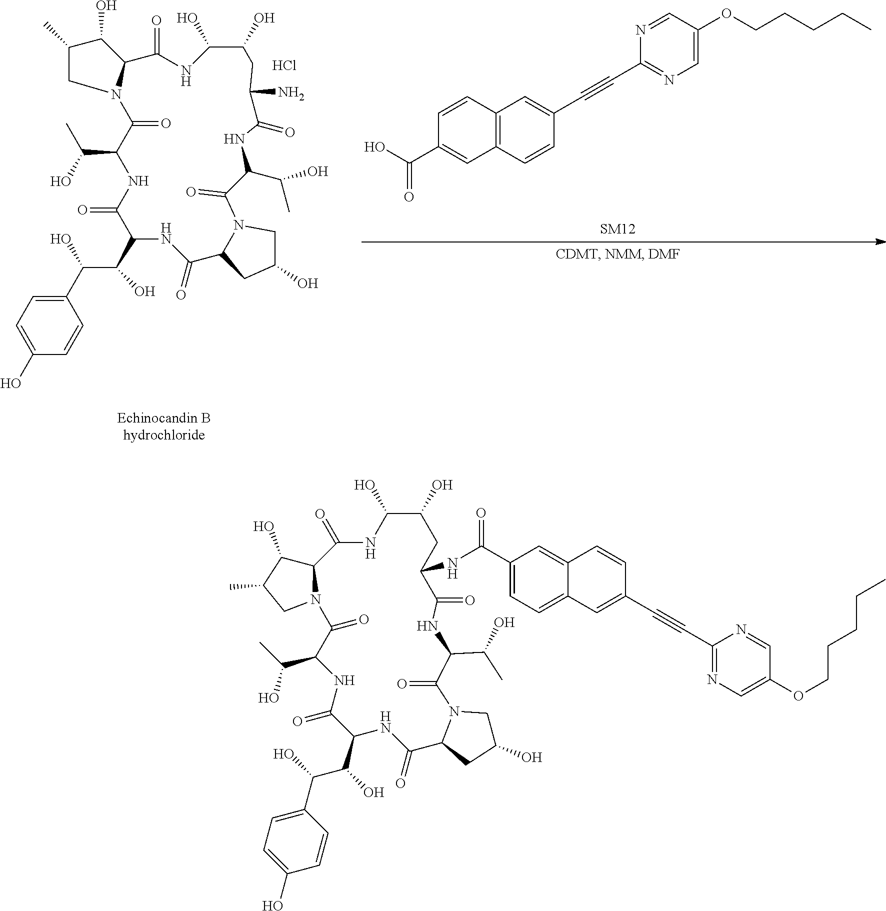

Echinocandin B hydrochloride (313 mg, 0.37 mmol, 1 eq.) was dissolved in DMF (4 mL), and SM12 (135 mg, 0.37 mmol, 1 eq.), NMM (0.12 mL, 1.12 mmol, 3 eq.) and CDMT (79 mg, 0.45 mmol, 1.2 eq.) were sequentially added. The reaction was performed for 6 h at room temperature, and 23.5 mg of a product was obtained through prep-HPLC purification, with a purity of 95% and a yield of 5%. HRMS[M−H]⁻: 1138.3973.

¹H NMR (400 MHz, CD₃OD) δ 8.47-8.39 (m, 2H), 8.24 (s, 1H), 8.06-7.95 (m, 3H), 7.72 (d, J=8.6 Hz, 1H), 7.58 (d, J=9.2 Hz, 1H), 7.15 (d, J=8.5 Hz, 2H), 6.76 (d, J=8.5 Hz, 2H), 5.37 (d, J=8.8 Hz, 1H), 5.02 (d, J=5.4 Hz, 2H), 4.73-4.66 (m, 1H), 4.64-4.54 (m, 3H), 4.39-4.30 (m, 3H), 4.26-4.16 (m, 5H), 4.08 (s, 1H), 3.99 (d, J=8.2 Hz, 1H), 3.85 (dd, J=22.6, 9.0 Hz, 2H), 3.45-3.38 (m, 2H), 2.50 (ddd, J=36.4, 17.3, 6.5 Hz, 2H), 2.25 (dd, J=18.5, 4.2 Hz, 1H), 2.18-2.03 (m, 2H), 1.90-1.82 (m, 2H), 1.55-1.39 (m, 4H), 1.27 (dd, J=10.4, 6.4 Hz, 6H), 1.07 (d, J=6.9 Hz, 3H), 0.97 (t, J=7.1 Hz, 3H).

### Example 7

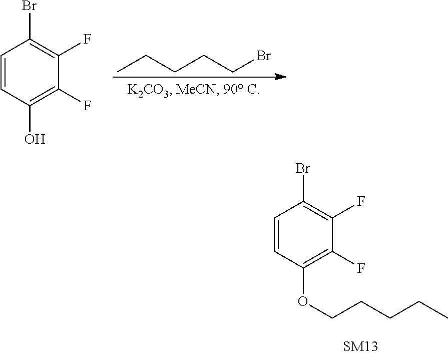

4-Bromo-2,3-difluorophenol (0.35 mL, 4.70 mmol, 1 eq.), bromopentane (0.7 ml, 5.70 mmol, 1.2 eq.) and potassium carbonate (2 g, 14.00 mmol, 3 eq.) were dissolved in acetonitrile (47 mL), and the mixture was heated, refluxed, and stirred at 90° C. After 1.5 h, TLC showed the formation of a new spot and the disappearance of the raw materials. The solvent was removed, ethyl acetate and water were added for extraction, the organic phases were combined, dried with anhydrous sodium sulfate, and concentrated to obtain 1.2 g of compound SM13 as a black oil, with a yield of 92%.

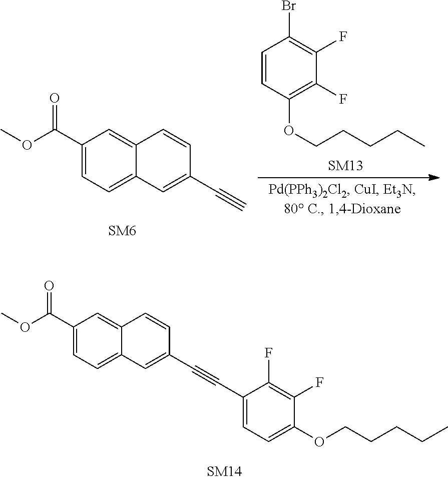 [SMILES: C#Cc1ccc2cc(C(=O)OC)ccc2c1 ; CCCCCOc1ccc(Br)c(F)c1F ; CCCCCOc1ccc(C#Cc2ccc3cc(C(=O)OC)ccc3c2)c(F)c1F]

SM6 (378 mg, 1.80 mmol, 1 eq.), SM13 (553 mg, 2.00 mmol, 1.1 eq.) and CuI (14 mg, 0.18 mmol, 0.1 eq.) were dissolved in 1,4-dioxane (7 mL), and then triethylamine (0.3 mL, 5.50 mmol, 3 eq.) was added. Vacuumizing and replacement with nitrogen were performed and repeated three times. Pd(PPh₃)₂Cl₂ (126 mg, 0.18 mmol, 0.1 eq.) was added under nitrogen atmosphere. The resulting mixture was heated and stirred overnight at 80° C. TLC showed a clear new spot. The solvent was removed. DCM was added for dissolving, and column chromatography purification was performed to obtain 101 mg of compound SM14 as a white solid, with a yield of 11%.

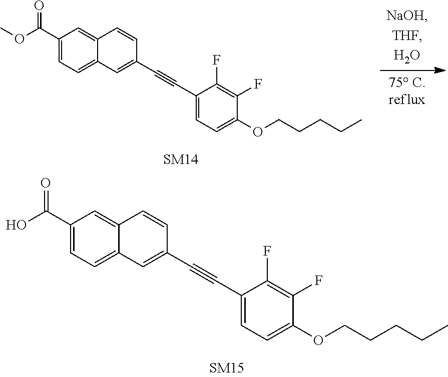 [SMILES: CCCCCOc1ccc(C#Cc2ccc3cc(C(=O)OC)ccc3c2)c(F)c1F ; CCCCCOc1ccc(C#Cc2ccc3cc(C(=O)O)ccc3c2)c(F)c1F]

SM14 (101 mg, 0.24 mmol, 1 eq.) was dissolved in THF (2 mL), and heated and stirred at 60° C., and an aqueous solution (0.5 mL) of NaOH (50 mg, 1.23 mmol, 5 eq.) was added to the reaction system. The reaction system was heated at 75° C. and refluxed overnight. After the reaction was completed, 2M HCl (aq.) was added to adjust the pH until an acidic state is reached, and then a solid was precipitated. Suction filtration was performed to obtain 75 mg of compound SM15 as a white solid, with a yield of 76%. MS[M−H]⁻: 393.

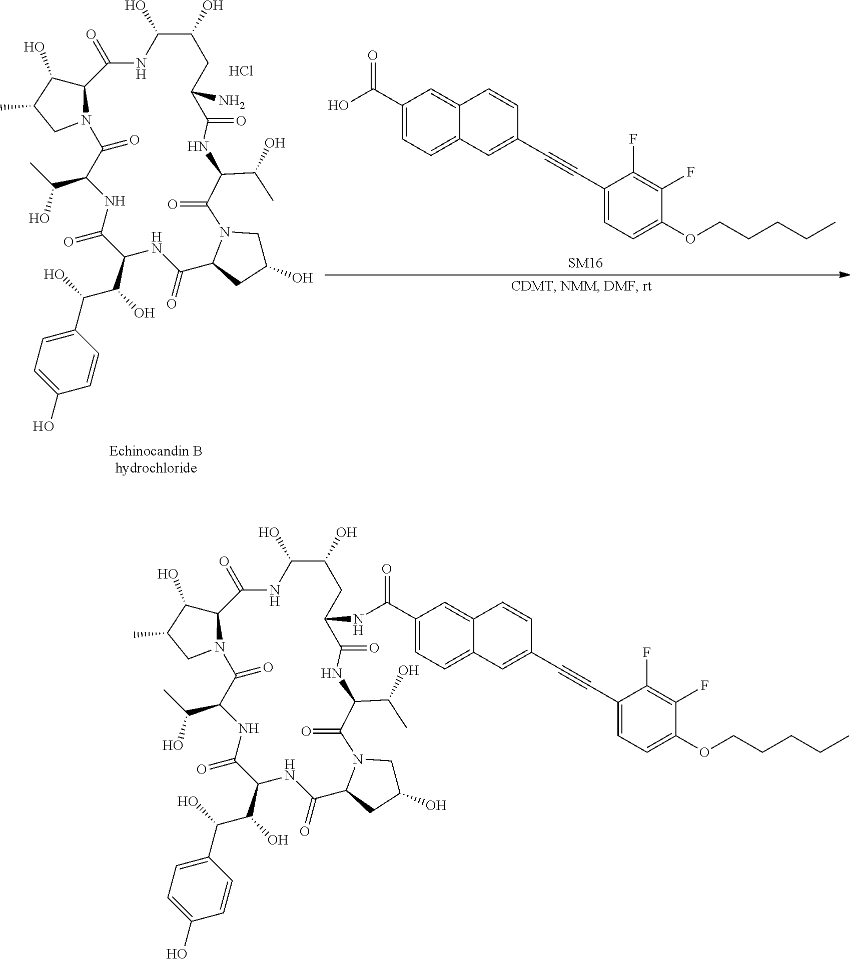 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1ccc(C#Cc2ccc3cc(C(=O)O)ccc3c2)c(F)c1F ; CCCCCOc1ccc(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)c(F)c1F]

Echinocandin B hydrochloride (159 mg, 0.19 mmol, 1 eq.), SM15 (75 mg, 0.19 mmol, 1 eq.) and CDMT (40 mg, 0.22 mmol, 1.2 eq.) were dissolved in DMF (2 mL), and then NMM (0.06 mL, 0.57 mmol, 3 eq.) was added. Stirring was performed for 1 h at room temperature. After the reaction was completed, the reaction solution was subjected to prep-HPLC purification and separation to obtain 128 mg of a compound as a white solid, with a purity of 96% and a yield of 57%. HRMS[M−H]⁻: 1172.4577.

¹H NMR (400 MHz, CD₃OD) δ 8.41 (s, 1H), 8.09 (s, 1H), 8.00-7.90 (m, 3H), 7.62 (dd, J=8.5, 1.4 Hz, 1H), 7.35-7.28 (m, 1H), 7.15 (d, J=8.5 Hz, 2H), 6.95 (t, J=7.5 Hz, 1H), 6.76 (d, J=8.5 Hz, 2H), 5.37 (d, J=2.9 Hz, 1H), 5.03 (d, J=3.3 Hz, 1H), 4.92 (s, 1H), 4.70 (dd, J=11.9, 5.2 Hz, 1H), 4.59 (dd, J=14.6, 7.2 Hz, 3H), 4.34 (dd, J=18.0, 5.3 Hz, 3H), 4.28-4.19 (m, 3H), 4.12 (t, J=6.4 Hz, 3H), 3.99 (d, J=8.1 Hz, 1H), 3.92-3.80 (m, 2H), 3.44-3.37 (m, 1H), 2.58-2.41 (m, 2H), 2.25 (t, J=8.5 Hz, 1H), 2.18-2.03 (m, 2H), 1.88-1.80 (m, 2H), 1.46 (ddd, J=22.2, 12.3, 6.9 Hz, 4H), 1.28 (dd, J=13.3, 6.3 Hz, 6H), 1.06 (d, J=6.9 Hz, 3H), 0.97 (t, J=7.1 Hz, 3H).

### Example 8

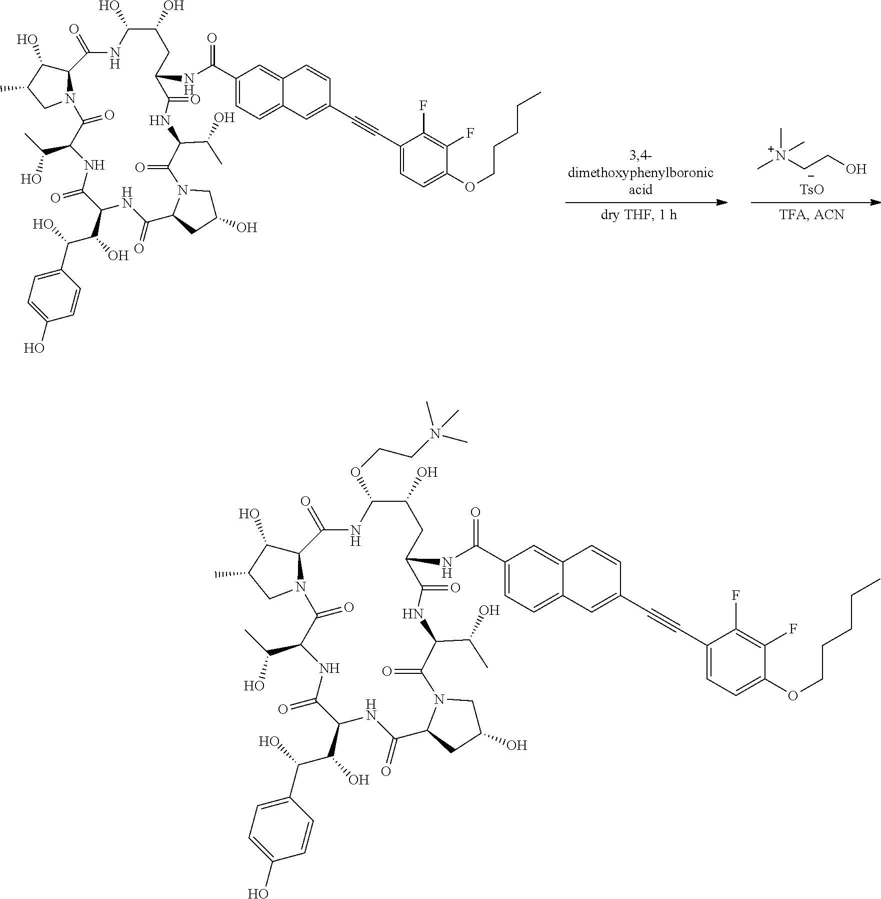

Example 7 (347 mg, 0.29 mmol, 1 eq.) and 3,4-dimethoxyphenylboronic acid (70 mg, 0.38 mmol, 1.3 eq.) were dissolved in dry THF (6 mL). The resulting solution was stirred for 1 h at room temperature, and concentrated to dryness. Choline tosylate (2.4 g, 8.80 mmol, 30 eq.) was added, then a mixed solution of TFA (0.85 mL) and acetonitrile (4.5 mL) was added to dissolve the reactants, and stirring was performed for 6 h at room temperature under N₂ atmosphere. After the reaction was completed, an aqueous solution of sodium acetate was added for quenching, and 74 mg of a compound (acetate) as a white solid was obtained through prep-HPLC purification, with a purity of 95% and a yield of 19%. HRMS[M]⁺: 1259.5431.

¹H NMR (400 MHz, CD₃OD) δ 8.46 (s, 1H), 8.14 (s, 1H), 8.00 (dd, J=15.9, 5.8 Hz, 3H), 7.66 (dd, J=8.5, 1.5 Hz, 1H), 7.23 (d, J=8.0 Hz, 1H), 7.15 (d, J=8.6 Hz, 2H), 6.96 (dd, J=12.0, 4.5 Hz, 1H), 6.76 (d, J=8.5 Hz, 2H), 5.42 (d, J=2.5 Hz, 1H), 5.04 (d, J=3.3 Hz, 1H), 4.79 (dd, J=12.0, 5.0 Hz, 2H), 4.58 (t, J=5.1 Hz, 3H), 4.39 (d, J=4.3 Hz, 1H), 4.35-4.31 (m, 2H), 4.25 (td, J=7.1, 3.1 Hz, 2H), 4.20-4.16 (m, 1H), 4.12 (dd, J=12.6, 6.1 Hz, 3H), 4.00 (d, J=11.3 Hz, 2H), 3.91 (dd, J=9.8, 7.0 Hz, 2H), 3.83 (d, J=10.9 Hz, 1H), 3.60 (d, J=4.8 Hz, 1H), 3.47 (d, J=7.0 Hz, 1H), 3.13 (s, 9H), 2.53-2.44 (m, 2H), 2.29 (d, J=8.9 Hz, 1H), 2.10-2.04 (m, 2H), 1.87-1.83 (m, 2H), 1.50-1.41 (m, 4H), 1.27 (d, J=6.3 Hz, 6H), 1.08 (d, J=6.9 Hz, 3H), 0.97 (s, 3H).

### Example 9

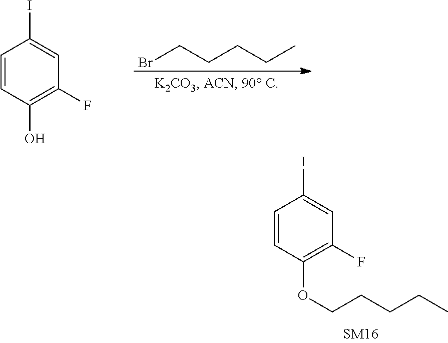

2-Fluoro-4-iodophenol (1 g, 4.20 mmol, 1 eq.) was dissolved in acetonitrile (20 mL), bromopentane (1.56 mL, 12.60 mmol, 3 eq.) was added, and potassium carbonate (1.743 g, 12.60 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 1.237 g of compound SM16 as a yellow liquid, with a yield of 95%.

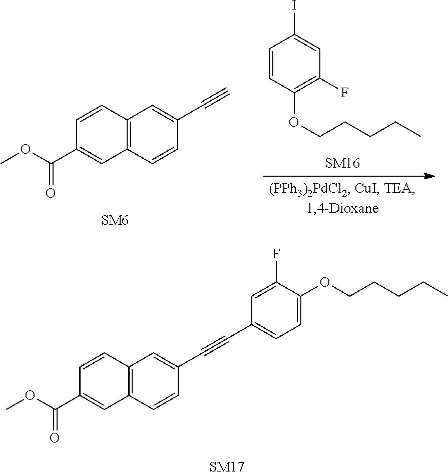 [SMILES: C#Cc1ccc2cc(C(=O)OC)ccc2c1 ; CCCCCOc1ccc(I)cc1F ; CCCCCOc1ccc(C#Cc2ccc3cc(C(=O)OC)ccc3c2)cc1F]

SM16 (293 mg, 0.95 mmol, 1 eq.) was dissolved in dioxane (10 mL), and replacement with nitrogen was performed. Under the protection of nitrogen, SM6 (200 mg, 0.95 mmol, 1 eq.), Pd(PPh₃)₂Cl₂ (67 mg, 0.09 mmol, 0.1 eq.), CuI (73 mg, 0.38 mmol, 0.4 eq.) and triethylamine (0.4 mL, 2.85 mmol, 3 eq.) were sequentially added. The mixture was stirred overnight at room temperature. TLC showed that the reaction was finished. The solvent was removed. Extraction was performed with water and DCM. The solvent was removed to obtain a crude product, and the crude product was separated and purified by column chromatography to obtain 282 mg of compound SM17 as a white solid, with a yield of 76%.

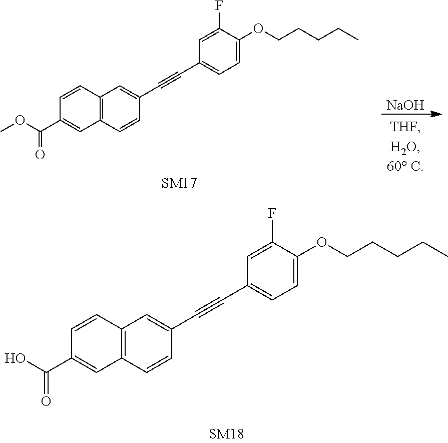 [SMILES: CCCCCOc1ccc(C#Cc2ccc3cc(C(=O)OC)ccc3c2)cc1F ; "NaOH" ; CCCCCOc1ccc(C#Cc2ccc3cc(C(=O)O)ccc3c2)cc1F]

SM17 (282 mg, 0.72 mmol, 1 eq.) was dissolved in THF (7 mL). Sodium hydroxide (123 mg, 2.89 mmol, 6 eq.) was dissolved in water (1 mL) and added to the system. The system was stirred at 60° C. for 5 h. TLC showed that the reaction was finished. The solvent was removed. Extraction was performed with water and DCM for the first time. Dilute hydrochloric acid was added to the aqueous phase to adjust the pH until an acidic state is reached. The aqueous phase was extracted with DCM, and the solvent was removed to obtain 255 mg of crude product SM18 as a white solid, with a yield of 94%.

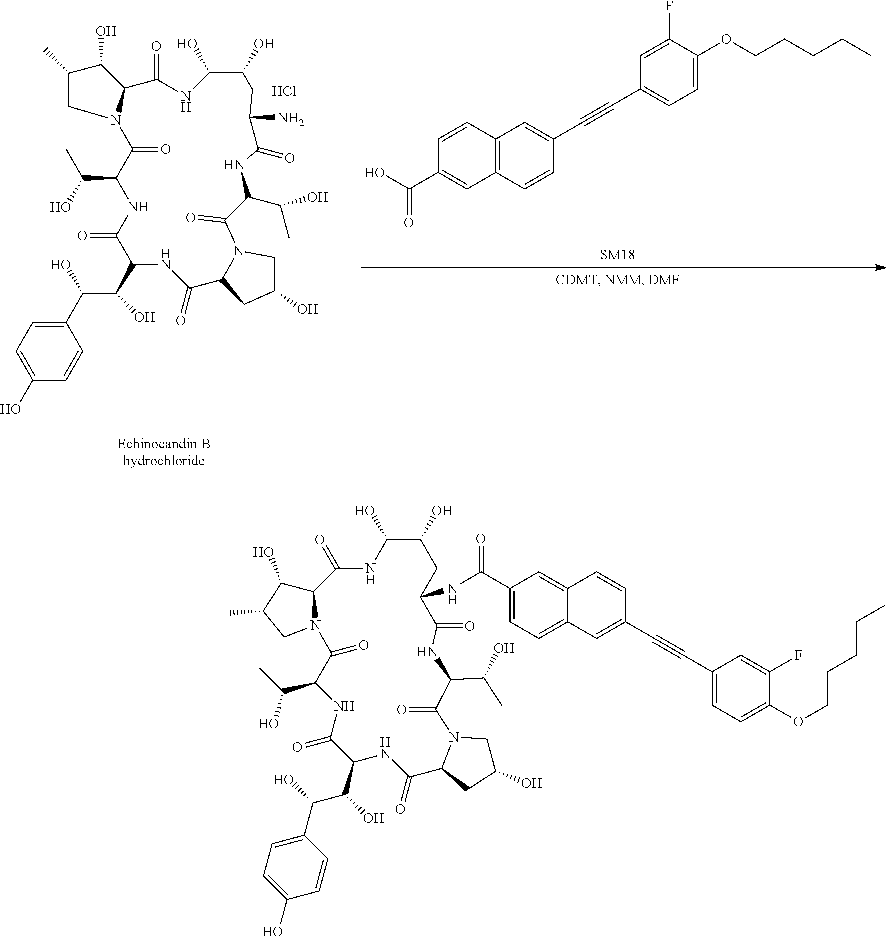

Echinocandin B hydrochloride (366 mg, 0.43 mmol, 1.2 eq.) was dissolved in DMF (5 mL), and SM18 (138 mg, 0.36 mmol, 1 eq.), NMM (0.13 mL, 1.09 mmol, 3 eq.) and CDMT (77 mg, 0.43 mmol, 1.2 eq.) were sequentially added. The reaction was performed for 6 h at room temperature, and 180 mg of a product was obtained through prep-HPLC purification, with a purity of 95% and a yield of 35%. HRMS[M+Na]⁺: 1178.4604.

¹H NMR (400 MHz, CD₃OD) δ 8.41 (d, J=10.8 Hz, 1H), 8.06 (s, 1H), 7.92 (dd, J=14.1, 8.5 Hz, 3H), 7.69 (d, J=9.5 Hz, 1H), 7.58 (d, J=6.8 Hz, 2H), 7.31 (dd, J=14.6, 4.8 Hz, 2H), 7.15 (t, J=7.1 Hz, 3H), 6.76 (d, J=8.5 Hz, 2H), 5.37 (d, J=2.8 Hz, 1H), 5.04 (d, J=5.2 Hz, 2H), 4.70 (dd, J=11.9, 5.1 Hz, 1H), 4.59 (dd, J=14.7, 7.5 Hz, 3H), 4.34 (dd, J=18.2, 5.3 Hz, 3H), 4.27-4.18 (m, 3H), 4.09 (t, J=6.4 Hz, 3H), 3.99 (d, J=8.3 Hz, 1H), 3.92-3.79 (m, 2H), 3.41 (t, J=9.0 Hz, 1H), 2.57-

2.41 (m, 2H), 2.25 (s, 1H), 2.11 (dd, J=16.3, 10.0 Hz, 2H), 1.87-1.78 (m, 2H), 1.51-1.40 (m, 4H), 1.28 (dd, J=13.4, 6.3 Hz, 6H), 1.06 (d, J=6.9 Hz, 3H), 0.97 (t, J=7.2 Hz, 3H).

### Example 10

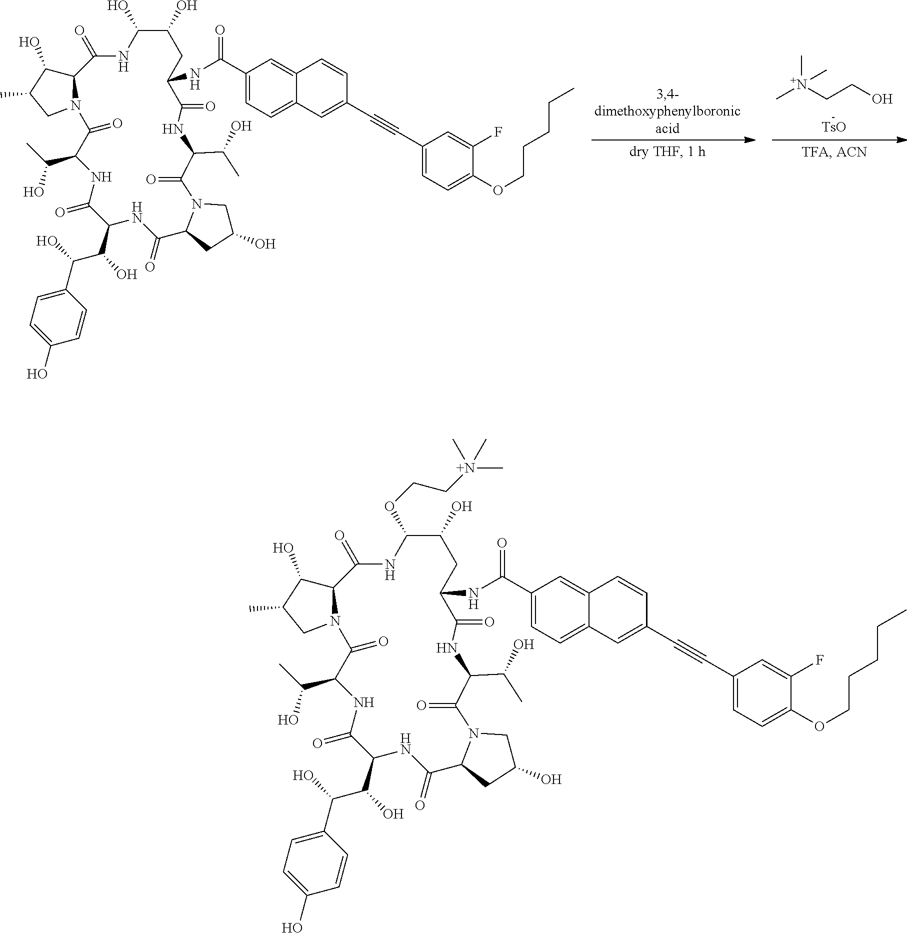

Example 9 (100 mg, 0.08 mmol, 1 eq.) and 3,4-dimethoxyphenylboronic acid (20 mg, 0.11 mmol, 1.3 eq.) were dissolved in dry THF (1.5 mL). The resulting solution was stirred for 1 h at room temperature, and concentrated to dryness. Choline tosylate (238 g, 0.86 mmol, 10 eq.) was added, then a mixed solution of TFA (0.25 mL) and acetonitrile (1 mL) was added to dissolve the reactants, and stirring was performed for 6 h at room temperature under N₂ atmosphere. After the reaction was completed, an aqueous solution of sodium acetate was added for quenching, and 27.6 mg of a compound (hydrochloride) as a white solid was obtained through prep-HPLC purification, with a purity of 96% and a yield of 25%. HRMS[M]⁺: 1241.5522.

¹H NMR (400 MHz, CD₃OD) δ 8.45 (s, 1H), 8.11 (s, 1H), 8.02-7.96 (m, 3H), 7.67-7.63 (m, 1H), 7.34-7.29 (m, 2H), 7.14 (t, J=8.8 Hz, 3H), 6.76 (d, J=8.5 Hz, 2H), 5.42 (d, J=2.4 Hz, 1H), 5.04 (d, J=3.2 Hz, 1H), 4.79 (dd, J=12.1, 5.1 Hz, 1H), 4.59 (dd, J=10.6, 7.4 Hz, 3H), 4.39 (d, J=4.3 Hz, 1H), 4.33 (d, J=8.3 Hz, 2H), 4.25 (dd, J=8.3, 6.4 Hz, 2H), 4.20-4.15 (m, 1H), 4.10 (t, J=6.5 Hz, 3H), 4.00 (d, J=11.4 Hz, 2H), 3.94-3.87 (m, 2H), 3.83 (d, J=10.7 Hz, 1H), 3.65-3.52 (m, 2H), 3.51-3.46 (m, 1H), 3.13 (s, 9H), 2.55-2.42 (m, 2H), 2.34-2.27 (m, 1H), 2.08 (dd, J=24.0, 4.3 Hz, 2H), 1.86-1.80 (m, 2H), 1.51-1.41 (m, 4H), 1.27 (d, J=6.3 Hz, 6H), 1.08 (d, J=6.9 Hz, 3H), 0.97 (t, J=7.1 Hz, 3H).

### Example 11

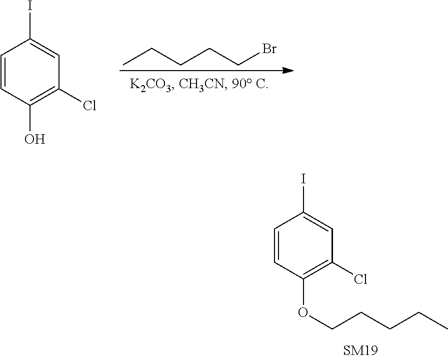

4-Iodo-2-chlorophenol (1 g, 3.90 mmol, 1 eq.), bromopentane (0.6 ml, 4.70 mmol, 1.2 eq.) and potassium carbonate (1.6 g, 11.00 mmol, 3 eq.) were dissolved in acetonitrile (40 mL), and the mixture was heated, refluxed, and stirred at 90° C. After 1.5 h, TLC showed the formation of a new spot and the disappearance of the raw materials. The solvent was removed, ethyl acetate and water were added for extraction, the organic phases were combined, dried with anhydrous sodium sulfate, and concentrated to obtain 1.0 g of compound SM19 as a yellow oil, with a yield of 78%.

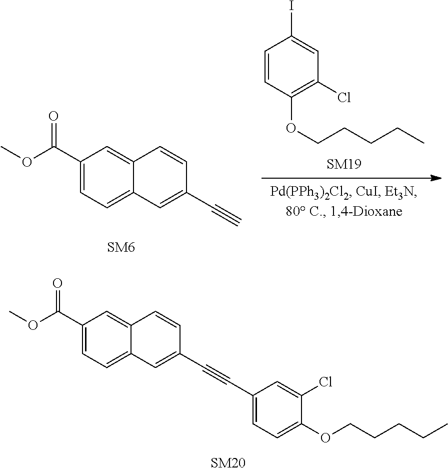 [SMILES: C#Cc1ccc2cc(C(=O)OC)ccc2c1 ; CCCCCOc1ccc(I)cc1Cl ; CCCCCOc1ccc(C#Cc2ccc3cc(C(=O)OC)ccc3c2)cc1Cl]

SM6 (432 mg, 2.00 mmol, 1 eq.), SM19 (800 mg, 2.40 mmol, 1.0 eq.) and CuI (39 mg, 0.20 mmol, 0.1 eq.) were dissolved in 1,4-dioxane (24 mL), and then triethylamine (0.8 mL, 6.10 mmol, 3 eq.) was added. Vacuumizing and replacement with nitrogen were performed and repeated three times. Pd(PPh₃)₂Cl₂ (144 mg, 0.20 mmol, 0.1 eq.) was added under nitrogen atmosphere. The resulting mixture was heated and stirred overnight at 80° C. TLC showed a clear new spot and the weak signals of raw materials SM3 and SM25. The solvent was removed. DCM was added for dissolving, and column chromatography purification was performed to obtain 261 mg of compound SM20 as a white solid, with a yield of 25%.

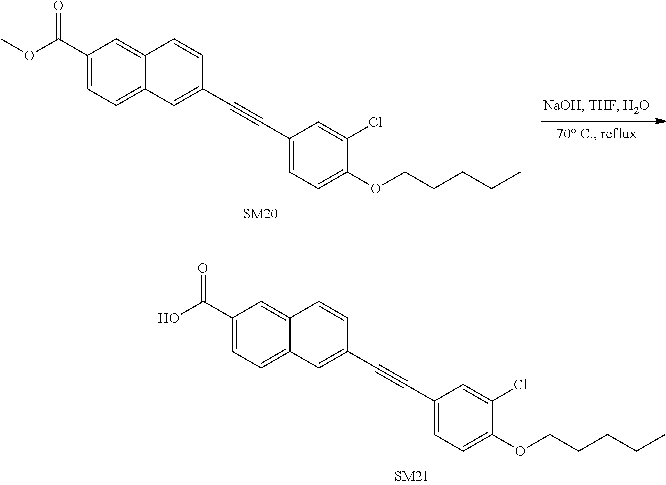 [SMILES: CCCCCOc1ccc(C#Cc2ccc3cc(C(=O)OC)ccc3c2)cc1Cl ; CCCCCOc1ccc(C#Cc2ccc3cc(C(=O)O)ccc3c2)cc1Cl]

SM20 (261 mg, 0.64 mmol, 1 eq.) was dissolved in THF (7 mL), and heated and stirred at 70° C., and then an aqueous solution (1.5 mL) of NaOH (129 mg, 3.20 mmol, 5 eq.) was added to the reaction system. The reaction system was heated at 70° C. and refluxed overnight. After the reaction was completed, 2M HCl (aq.) was added to adjust the pH value until an acidic state is reached, and then a solid was precipitated. Suction filtration was performed to obtain 233 mg of compound SM21 as a white solid, with a yield of 92%.

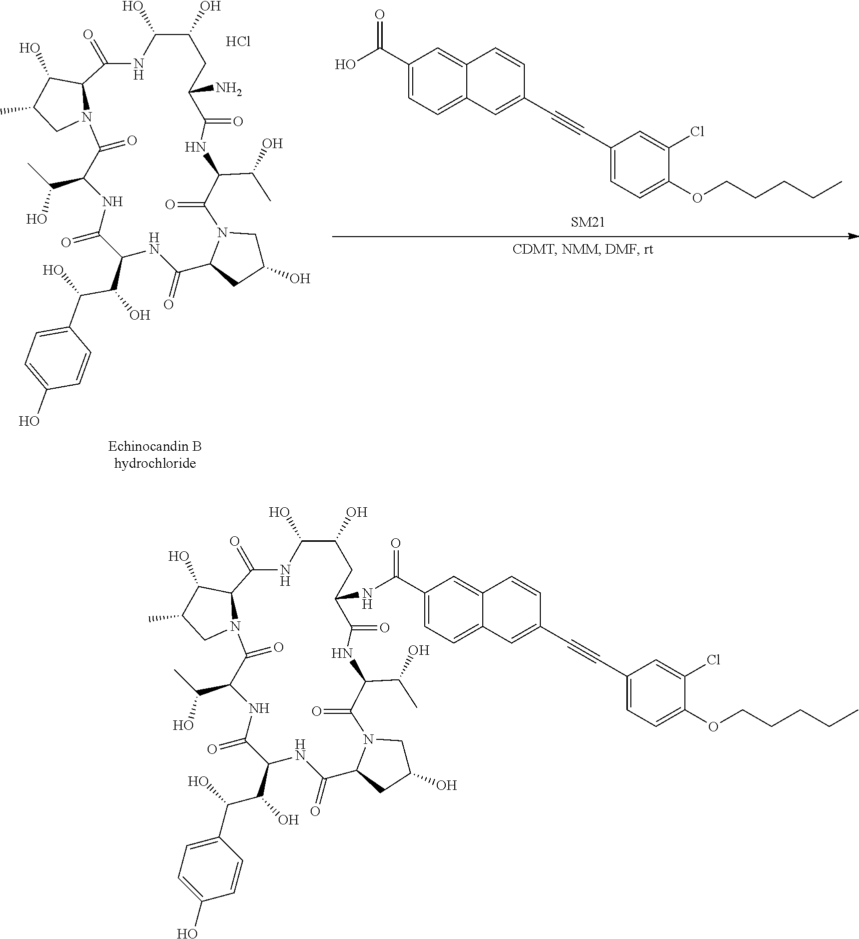 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1ccc(C#Cc2ccc3cc(C(=O)O)ccc3c2)cc1Cl ; CCCCCOc1ccc(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1Cl]

Echinocandin B hydrochloride (150 mg, 0.17 mmol, 1 eq.), SM21 (75 mg, 0.17 mmol, 1 eq.) and CDMT (38 mg, 0.22 mmol, 1.2 eq.) were dissolved in DMF (1.8 mL), and then NMM (0.06 mL, 0.53 mmol, 3 eq.) was added. Stirring was performed for 1 h at room temperature. After the reaction was completed, the reaction solution was subjected to prep-HPLC purification and separation to obtain 138 mg of a compound as a white solid, with a purity of 96% and a yield of 65%. HRMS[M−H]⁻: 1170.4768.

¹H NMR (400 MHz, CD₃OD) δ 8.40 (s, 1H), 8.07 (s, 1H), 7.92 (dt, J=13.2, 8.7 Hz, 3H), 7.61-7.56 (m, 2H), 7.47 (dd, J=8.6, 2.0 Hz, 1H), 7.15 (d, J=8.5 Hz, 2H), 7.08 (d, J=8.7 Hz, 1H), 6.76 (d, J=8.5 Hz, 2H), 5.37 (d, J=2.9 Hz, 1H), 5.03 (d, J=3.2 Hz, 1H), 4.74-4.66 (m, 2H), 4.64-4.55 (m, 3H), 4.34 (dd, J=18.7, 5.3 Hz, 3H), 4.27-4.19 (m, 3H), 4.10 (t, J=6.3 Hz, 3H), 3.99 (d, J=8.1 Hz, 1H), 3.85 (dd, J=21.2, 9.1 Hz, 2H), 3.41 (t, J=9.1 Hz, 1H), 2.57-2.41 (m, 2H), 2.29-2.21 (m, 1H), 2.16-2.04 (m, 2H), 1.84 (dd, J=14.5, 6.5 Hz, 2H), 1.49 (ddd, J=24.5, 11.7, 5.2 Hz, 4H), 1.28 (dd, J=12.5, 6.3 Hz, 6H), 1.06 (d, J=6.9 Hz, 3H), 0.97 (t, J=7.2 Hz, 3H).

### Example 12

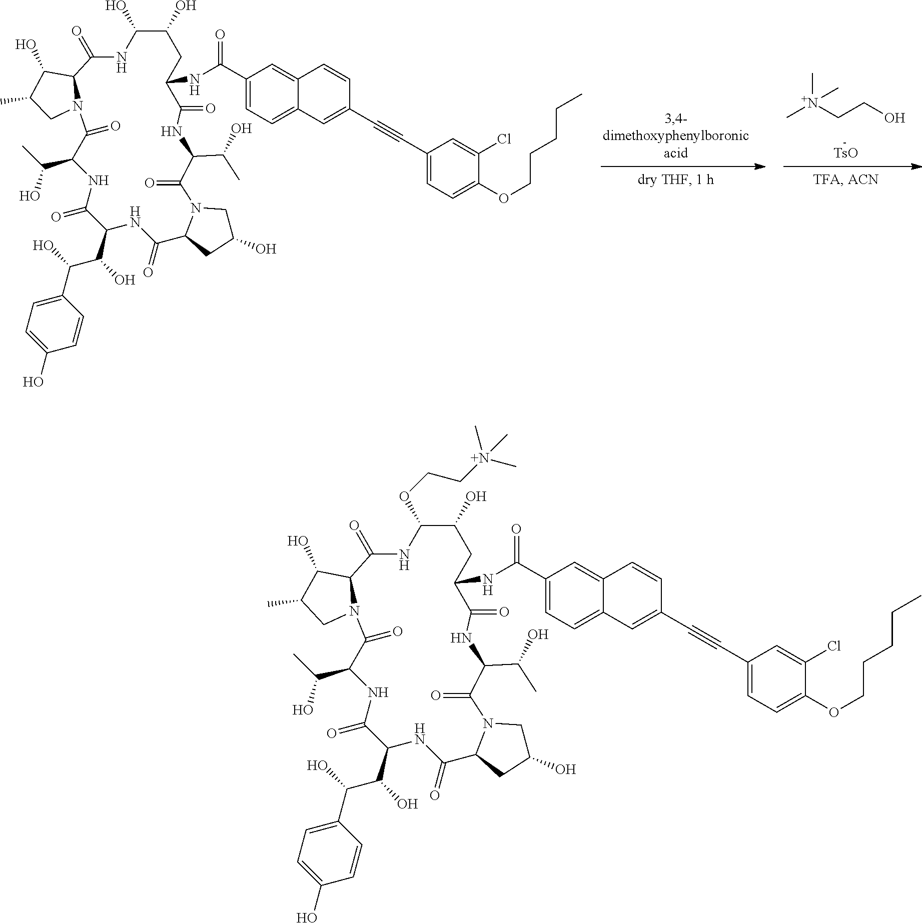

Example 11 (100 mg, 0.08 mmol, 1 eq.) and 3,4-dimethoxyphenylboronic acid (20 mg, 0.11 mmol, 1.3 eq.) were dissolved in dry THF (1.5 mL). The resulting solution was stirred for 1 h at room temperature, and concentrated to dryness. Choline tosylate (235 mg, 0.85 mmol, 10 eq.) was added, then a mixed solution of TFA (0.25 mL) and acetonitrile (1.5 mL) was added to dissolve the reactants, and stirring was performed for 6 h at room temperature under N₂ atmosphere. After the reaction was completed, an aqueous solution of sodium acetate was added for quenching, and 28 mg of a compound (hydrochloride) as a white solid was obtained through prep-HPLC purification, with a purity of 96% and a yield of 26%. HRMS[M]⁺: 1257.5352.

¹H NMR (400 MHz, CD₃OD) δ 8.45 (s, 1H), 8.11 (s, 1H), 8.05-7.96 (m, 3H), 7.65 (d, J=8.4 Hz, 1H), 7.58 (d, J=2.0 Hz, 1H), 7.48 (dd, J=8.5, 2.0 Hz, 1H), 7.15 (d, J=8.5 Hz, 2H), 7.09 (d, J=8.6 Hz, 1H), 6.76 (d, J=8.6 Hz, 2H), 5.42 (d, J=2.4 Hz, 1H), 5.04 (d, J=3.2 Hz, 1H), 4.90 (s, 1H), 4.81-4.75 (m, 1H), 4.58 (d, J=6.5 Hz, 3H), 4.39 (d, J=4.3 Hz, 1H), 4.33 (d, J=7.9 Hz, 2H), 4.29-4.23 (m, 2H), 4.21-4.15 (m, 1H), 4.11 (t, J=6.3 Hz, 3H), 4.00 (d, J=11.3 Hz, 1H), 3.93-3.88 (m, 1H), 3.83 (d, J=10.8 Hz, 1H), 3.68-3.44 (m, 4H), 3.13 (s, 9H), 2.56-2.41 (m, 2H), 2.35-2.25 (m, 1H), 2.08 (dd, J=15.3, 9.7 Hz, 2H), 1.88-1.81 (m, 2H), 1.49 (ddd, J=24.5, 11.6, 5.1 Hz, 4H), 1.27 (d, J=6.2 Hz, 6H), 1.08 (d, J=6.9 Hz, 3H), 0.97 (t, J=7.2 Hz, 3H).

### Example 13

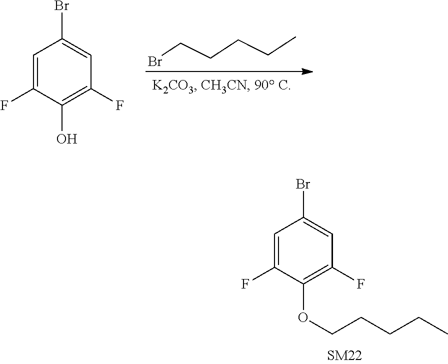

4-Bromo-2,6-difluorophenol (1 g, 4.78 mmol, 1 eq.) was dissolved in acetonitrile (20 mL), bromopentane (1.8 mL, 14.35 mmol, 3 eq.) was added, and potassium carbonate (1.984 g, 14.35 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 1.317 g of compound SM22 as a yellow liquid, with a yield of 99%.

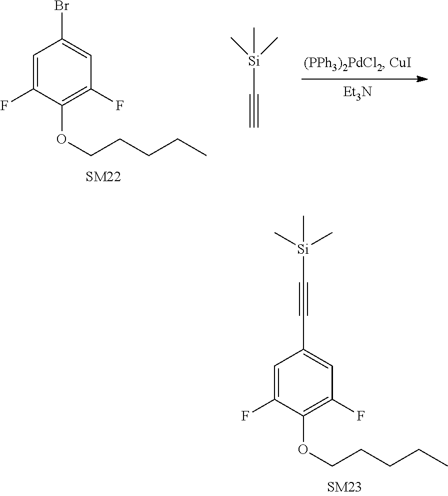

SM22 (1 g, 3.58 mmol, 1 eq.) was dissolved in triethylamine (17 mL), and trimethylsilylacetylene (0.51 mL, 3.58 mmol, 1 eq.), Pd(PPh₃)₂Cl₂ (252 mg, 0.35 mmol, 0.1 eq.) and CuI (68 mg, 0.35 mmol, 0.1 eq.) were added. Replacement with nitrogen was performed. The resulting mixture was stirred at 90° C. for 2 h in a microwave reactor. TLC showed the completion of the reaction and the formation of a new spot. Column chromatography separation and purification was performed to obtain 922 mg of compound SM23 as a yellow liquid, with a yield of 87%.

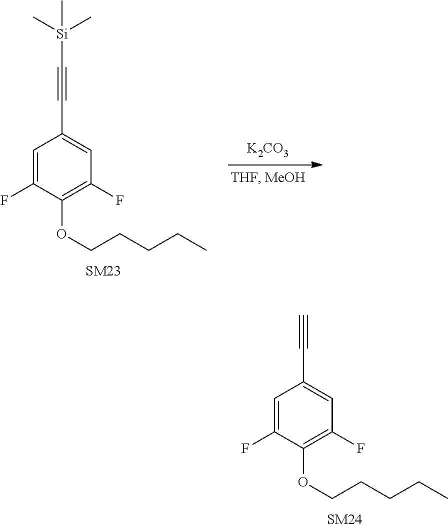

SM23 (922 mg, 3.11 mmol, 1 eq.) was dissolved in MeOH (10 mL) and THF (10 mL). Potassium carbonate (654 mg, 4.66 mmol, 1.5 eq.) was added. The mixture was stirred for 3 h at room temperature. TLC showed the completion of the reaction, the formation of a new spot, and the disappearance of the raw materials. The resulting mixture was extracted with water and DCM, and the solvent was removed to obtain 600 mg of crude product SM24 as a yellow oil, with a yield of 86%.

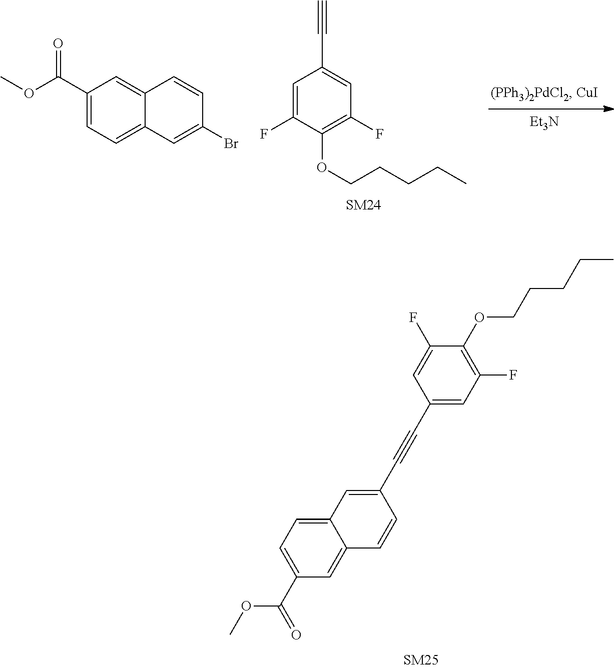

Methyl 6-bromo-2-naphthoate (639 mg, 2.41 mmol, 1 eq.) was dissolved in triethylamine (12 mL), and SM24 (540 mg, 2.41 mmol, 1 eq.), (PPh₃)₂PdCl₂ (169 mg, 0.24 mmol, 0.1 eq.) and CuI (46 mg, 0.24 mmol, 0.1 eq.) were added. Replacement with nitrogen was performed. The resulting mixture was reacted at 90° C. under microwave condition, and stirred for 2 h. TLC showed the formation of a new spot and the disappearance of the raw materials. Column chromatography separation and purification was performed to obtain 662 mg of compound SM25 as a yellow solid, with a yield of 67%.

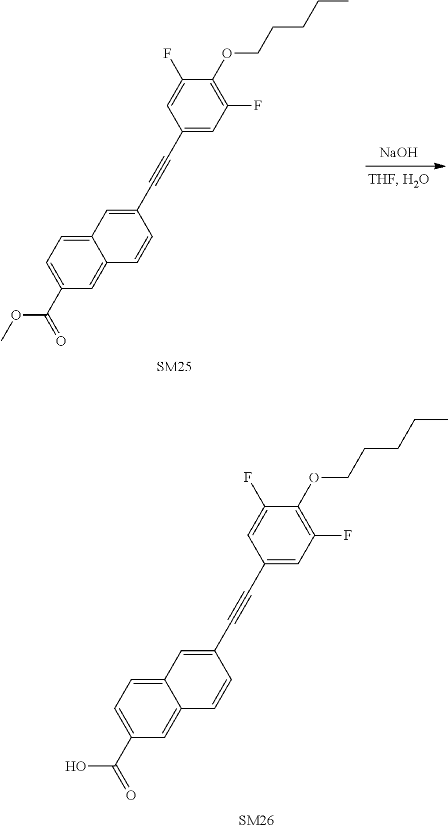

SM25 (662 mg, 1.62 mmol, 1 eq.) was dissolved in THF (9 mL). NaOH (259 mg, 6.48 mmol, 4 eq.) was dissolved in water (1 mL) and added to the reaction system. The reaction system was stirred overnight at 60° C. TLC showed a spot of product. After THF was subjected to rotary evaporation, extraction was performed with water and EA for the first time. The aqueous phase was remained for acidifying, and a solid was precipitated. Suction filtration and drying were performed to obtain 621 mg of compound SM26 as a yellow solid, with a yield of 97%.

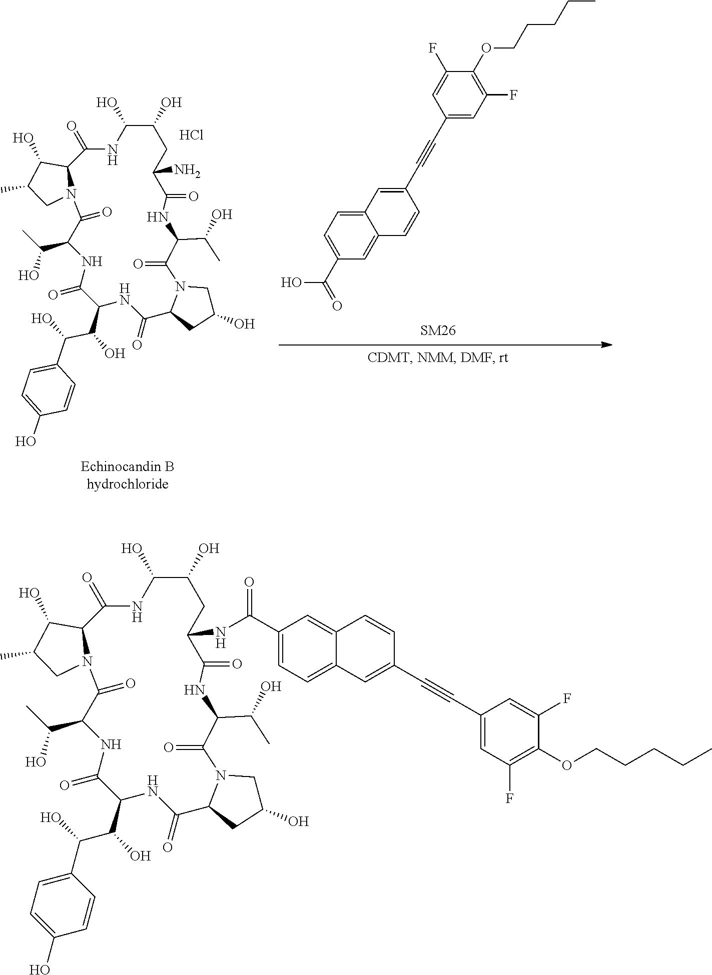 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCOc1c(F)cc(C#Cc2ccc3cc(C(=O)O)ccc3c2)cc1F ; CCCCCOc1c(F)cc(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F]

SM26 (236 mg, 0.59 mmol, 1 eq.) was dissolved in DMF (6 mL), echinocandin B hydrochloride (500 mg, 0.59 mmol, 1 eq.) and CDMT (126 mg, 0.71 mmol, 1.2 eq.) were sequentially added, and NMM (0.20 mL, 1.79 mmol, 3 eq.) was finally added. The mixture was stirred for 4 h at room temperature. 211 mg of a white solid was obtained through prep-HPLC purification, with a purity of 96% and a yield of 30%. HRMS[M+Na]⁺: 1196.4405.

¹H NMR (400 MHz, CD₃OD) δ 8.41 (s, 1H), 8.10 (s, 1H), 7.94 (dd, J=16.1, 7.7 Hz, 3H), 7.65-7.61 (m, 1H), 7.23 (d, J=8.4 Hz, 2H), 7.15 (d, J=8.5 Hz, 2H), 6.76 (d, J=8.5 Hz, 2H), 5.36 (s, 1H), 5.03 (s, 2H), 4.70 (dd, J 11.8, 5.1 Hz, 1H), 4.64-4.53 (m, 3H), 4.39-4.30 (m, 3H), 4.27-4.15 (m, 5H), 4.09 (s, 1H), 3.99 (d, J=8.4 Hz, 1H), 3.93-3.80 (m, 2H), 3.41 (t, J=9.1 Hz, 1H), 2.58-2.40 (m, 2H), 2.25 (s, 1H), 2.18-2.04 (m, 2H), 1.81-1.72 (m, 2H), 1.54-1.39 (m, 4H), 1.28 (dd, J=13.2, 6.2 Hz, 6H), 1.06 (d, J=6.9 Hz, 3H), 0.96 (t, J=7.2 Hz, 3H).

### Example 14

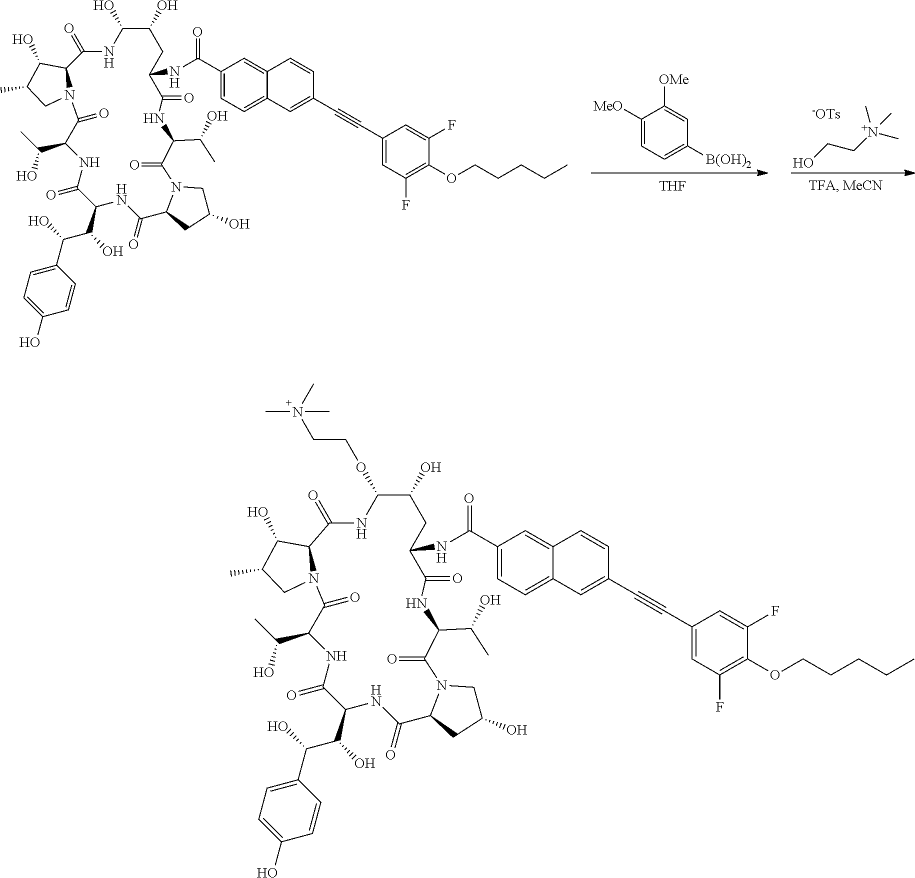

Example 13 (150 mg, 0.12 mmol, 1 eq.) was dissolved in dry THF (3 mL), and 3,4-dimethoxyphenylboronic acid (30 mg, 0.16 mmol, 1.3 eq.) was added. After the resulting mixture was stirred for 1 h at room temperature, the solvent was removed, and then dry THF (3 mL) was added for rotary evaporation. Choline tosylate (352 mg, 1.27 mmol, 10 eq.) was added, and TFA (0.38 mL) mixed with acetonitrile (3 mL) was added to the system. The system was stirred for 3 h at room temperature. LCMS showed the formation of a product. 57 mg of a white solid (acetate) was obtained through prep-HPLC purification, with a purity of 98% and a yield of 28%. HRMS[M]⁺: 1259.5494.

¹H NMR (400 MHz, CD₃OD) δ 8.45 (s, 1H), 8.14 (s, 1H), 8.02-7.97 (m, 3H), 7.66 (d, J=8.5 Hz, 1H), 7.23 (d, J=8.4 Hz, 2H), 7.15 (d, J=8.5 Hz, 2H), 6.76 (d, J=8.4 Hz, 2H), 5.42 (s, 1H), 5.05 (d, J=3.0 Hz, 1H), 4.81-4.77 (m, 1H), 4.60 (d, J=11.6 Hz, 4H), 4.39 (d, J=4.3 Hz, 1H), 4.34 (s, 2H), 4.25 (dd, J=10.0, 5.7 Hz, 3H), 4.19 (t, J=6.4 Hz, 3H), 4.11 (s, 1H), 4.00 (d, J=10.9 Hz, 2H), 3.94-3.88 (m, 2H), 3.83 (d, J=10.6 Hz, 1H), 3.50 (d, J=6.9 Hz, 1H), 3.13 (s, 9H), 2.54-2.43 (m, 2H), 2.30 (s, 1H), 2.07 (d, J=12.8 Hz, 2H), 1.90 (s, 3H), 1.79-1.74 (m, 2H), 1.48-1.39 (m, 4H), 1.27 (d, J=6.2 Hz, 6H), 1.08 (d, J=6.9 Hz, 3H), 0.96 (t, J=7.2 Hz, 3H).

### Example 15

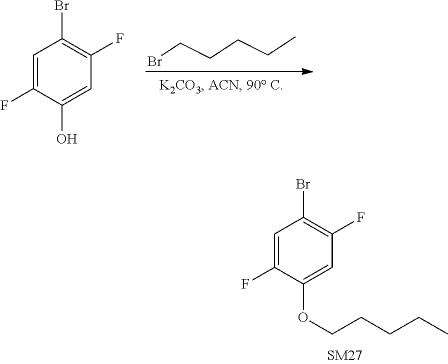

4-Bromo-2,5-difluorophenol (1 g, 4.78 mmol, 1 eq.) was dissolved in acetonitrile (25 mL), bromopentane (1.8 mL, 14.35 mmol, 3 eq.) was added, and potassium carbonate (1.984 g, 14.35 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 1.280 g of crude compound SM27 as a yellow liquid, with a yield of 96%.

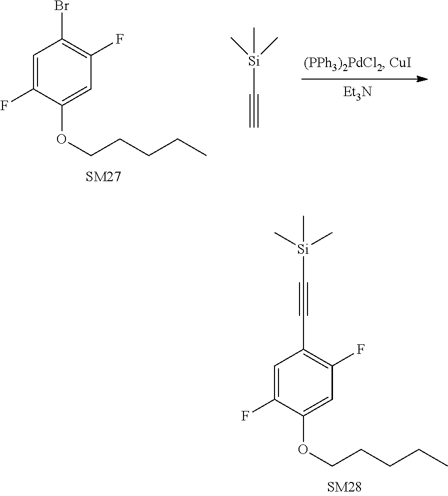

SM27 (500 g, 1.79 mmol, 1 eq.) was dissolved in triethylamine (15 mL), and the resulting solution was added into a sealed tube. Replacement with nitrogen was performed by bubbling. Trimethylsilylacetylene (0.38 mL, 2.68 mmol, 1.5 eq.), Pd(PPh₃)₂Cl₂ (126 mg, 0.17 mmol, 0.1 eq.) and CuI (34 mg, 0.17 mmol, 0.1 eq.) were added. The resulting mixture was stirred overnight in an oil bath at 90° C. TLC showed the completion of the reaction and the formation of a new spot. The solvent was removed, and the resulting product was dissolved in DCM. The organic phase was washed sequentially with an ammonium chloride solution, dilute hydrochloric acid and saturated brine. The solvent was removed to obtain a crude product. The crude product was separated and purified by column chromatography to obtain 537 mg of compound SM28 as a yellow oil, with a yield of 99%.

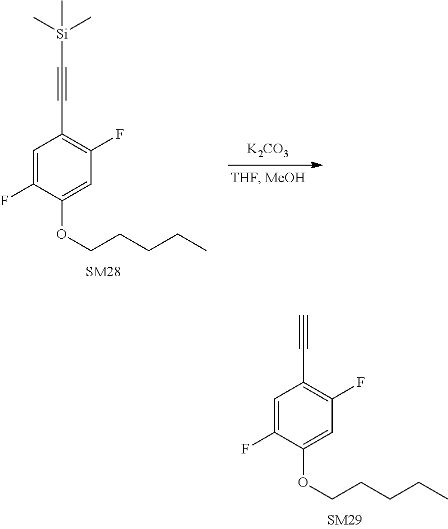

SM28 (537 mg, 1.81 mmol, 1 eq.) was dissolved in MeOH (9 mL) and THF (9 mL). Potassium carbonate (376 mg, 2.71 mmol, 1.5 eq.) was added. The resulting mixture was stirred for 3 h at room temperature. TLC showed the completion of the reaction, the formation of a new spot, and the disappearance of the raw materials. The resulting mixture was extracted with water and DCM, and the solvent was removed to obtain 402.5 mg of crude product SM29 as a yellow oil, with a yield of 99%.

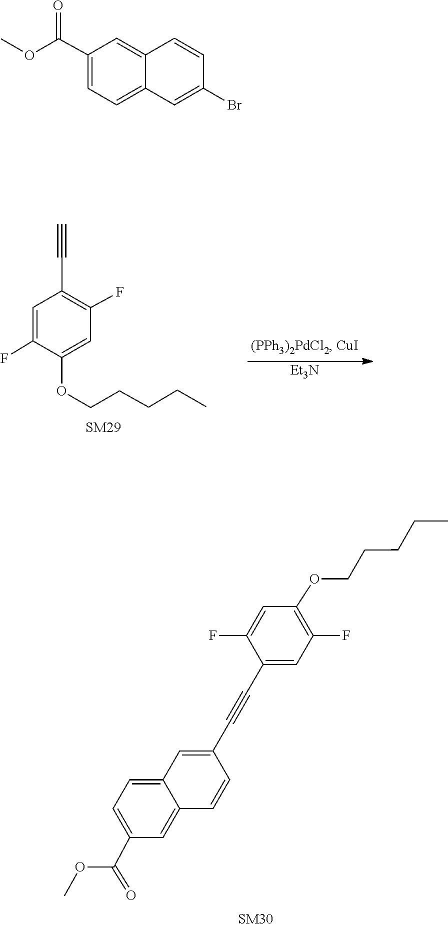 [SMILES: "Et3N" ; COC(=O)c1ccc2cc(Br)ccc2c1 ; C#Cc1cc(F)c(OCCCCC)cc1F ; CCCCCOc1cc(F)c(C#Cc2ccc3cc(C(=O)OC)ccc3c2)cc1F]

Methyl 6-bromo-2-naphthoate (592 mg, 2.23 mmol, 1 eq.) was dissolved in triethylamine (10 mL), and SM29 (500 mg, 2.23 mmol, 1 eq.), (PPh₃)₂PdCl₂ (157 mg, 0.22 mmol, 0.1 eq.) and CuI (43 mg, 0.22 mmol, 0.1 eq.) were added. Replacement with nitrogen was performed. The resulting mixture was stirred overnight in an oil bath at 90° C. TLC showed the formation of a new spot and the disappearance of the raw materials. The solvent was removed. The resulting mixture was extracted with water and DCM. The solvent was removed to obtain a crude product. The crude product was separated and purified by column chromatography to obtain 678 mg of compound SM30 as a yellow solid, with a yield of 74%.

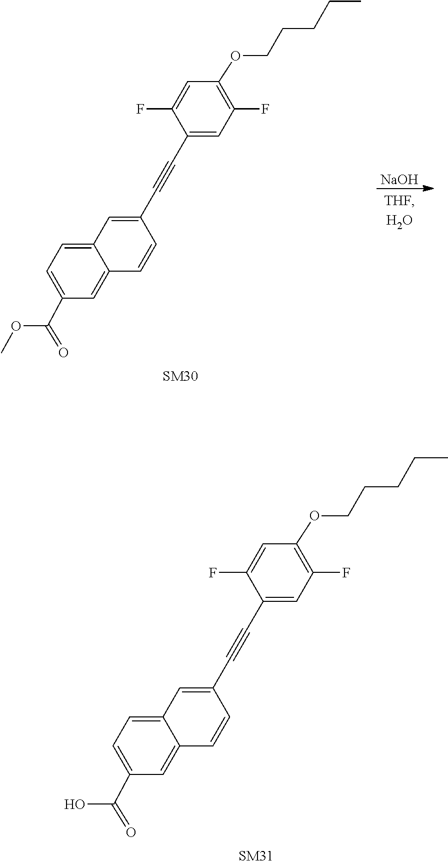 [SMILES: CCCCCOc1cc(F)c(C#Cc2ccc3cc(C(=O)OC)ccc3c2)cc1F ; "NaOH" ; CCCCCOc1cc(F)c(C#Cc2ccc3cc(C(=O)O)ccc3c2)cc1F]

SM30 (678 mg, 1.66 mmol, 1 eq.) was dissolved in THF (16 mL). NaOH (266 mg, 6.64 mmol, 4 eq.) was dissolved in water (2 mL) and added to the reaction system. The reaction system was stirred overnight at 60° C. TLC showed a spot of product. After THF was subjected to rotary evaporation, extraction was performed with water and DCM for the first time. The aqueous phase was remained for acidifying, extraction was performed with DCM, and the solvent was removed to obtain 418.6 mg of crude product SM31 as a yellow solid, with a yield of 64%.

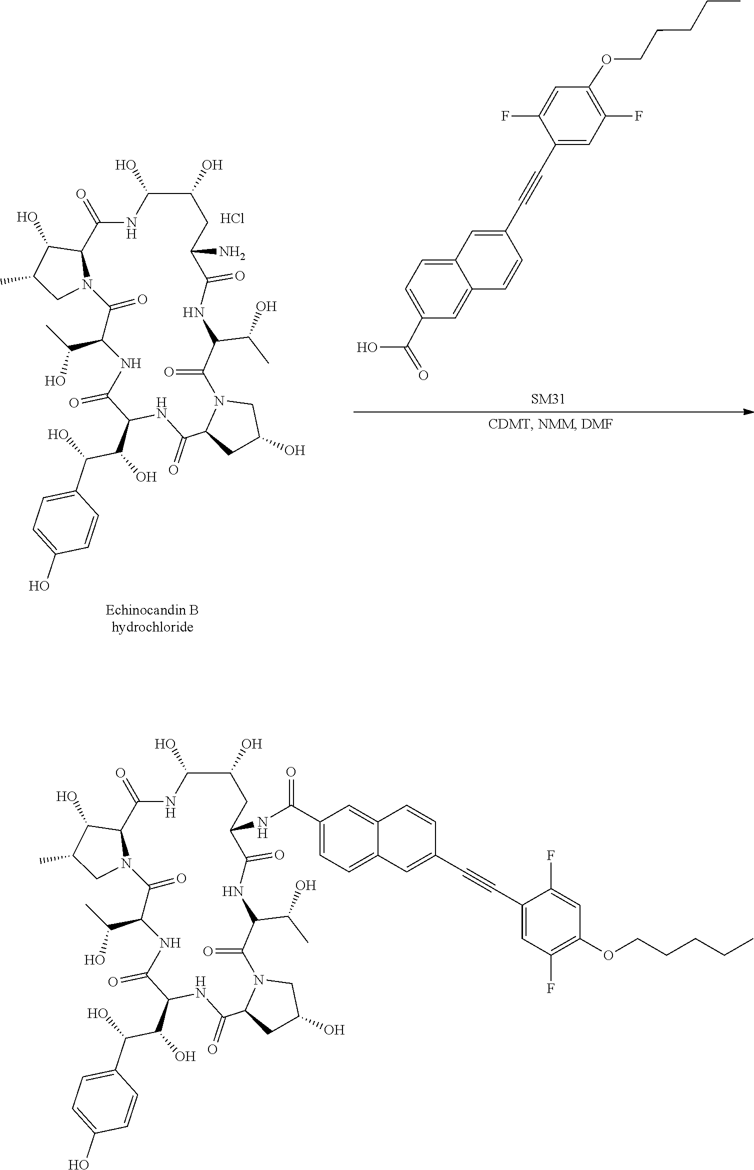

SM31 (71 mg, 0.18 mmol, 1 eq.) was dissolved in DMF (2 mL), echinocandin B hydrochloride (150 mg, 0.18 mmol, 1 eq.) and CDMT (38 mg, 0.21 mmol, 1.2 eq.) were sequentially added, and NMM (0.06 mL, 0.54 mmol, 3 eq.) was finally added. The resulting mixture was stirred for 4 h at room temperature. 83 mg of a compound as a white solid was obtained through prep-HPLC purification, with a purity of 96% and a yield of 39%. HRMS[M+Na]⁺: 1196.4617.

¹H NMR (400 MHz, CD₃OD) δ 8.42 (s, 1H), 8.10 (s, 1H), 8.01-7.92 (m, 3H), 7.62 (dd, J=8.5, 1.2 Hz, 1H), 7.33 (dd, J=11.1, 6.7 Hz, 1H), 7.15 (d, J=8.5 Hz, 2H), 7.02 (dd, J=10.7, 7.2 Hz, 1H), 6.76 (d, J=8.5 Hz, 2H), 5.36 (s, 1H), 5.02 (s, 2H), 4.69 (d, J=6.2 Hz, 1H), 4.61 (dd, J=14.9, 10.6 Hz, 3H), 4.34 (dd, J=18.9, 5.7 Hz, 3H), 4.26-4.18 (m, 3H), 4.09 (t, J=6.4 Hz, 3H), 3.99 (d, J=10.9 Hz, 1H), 3.88 (dd, J=18.5, 10.7 Hz, 2H), 3.43-3.38 (m, 1H), 2.60-2.39 (m, 2H), 2.30-2.20 (m, 1H), 2.18-2.03 (m, 2H), 1.88-1.80 (m, 2H), 1.52-1.39 (m, 4H), 1.28 (dd, J=11.6, 6.3 Hz, 6H), 1.06 (d, J=6.8 Hz, 3H), 0.97 (t, J=7.1 Hz, 3H).

### Example 16

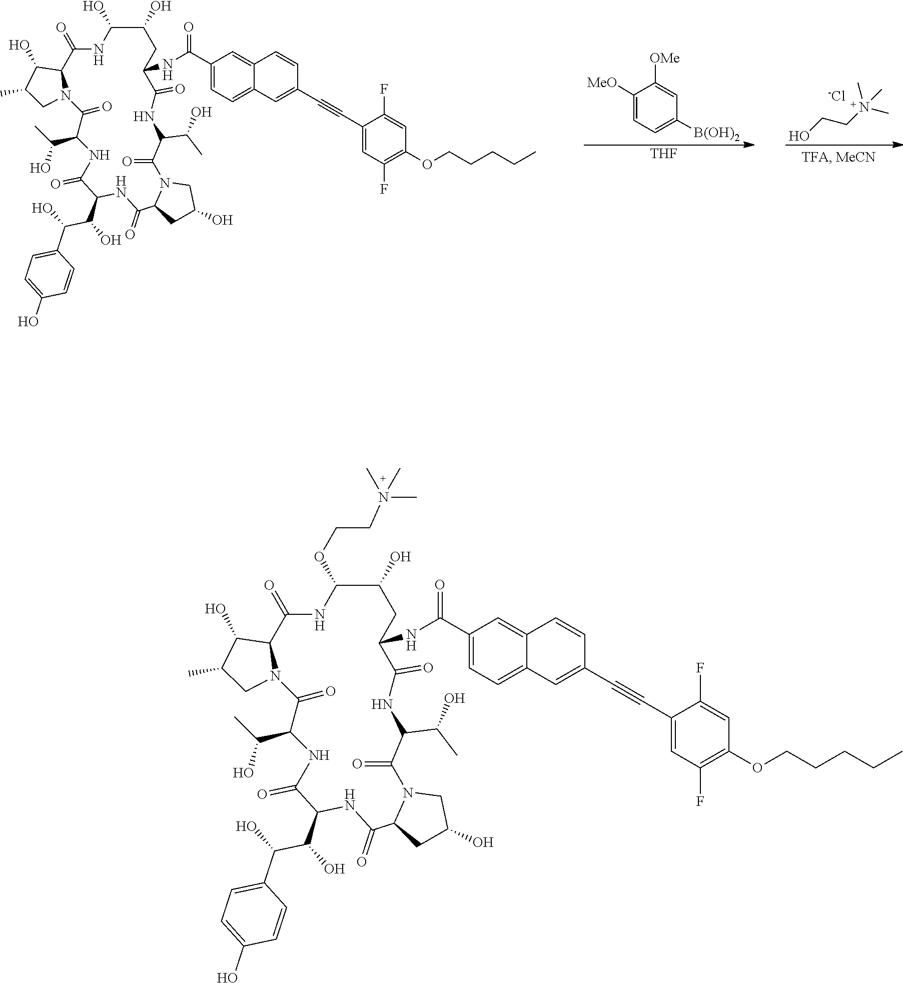

Example 15 (250 mg, 0.21 mmol, 1 eq.) was dissolved in dry THF (5 mL), and 3,4-dimethoxyphenylboronic acid (50 mg, 0.27 mmol, 1.3 eq.) was added. After the resulting mixture was stirred for 1 h at room temperature, the solvent was removed, and then dry THF (5 mL) was added for rotary evaporation. Choline chloride (297 mg, 2.12 mmol, 10 eq.) was added, and TFA (0.63 mL) mixed with acetonitrile (5 mL) was added to the system. The system was stirred for 3 h at room temperature. LCMS showed the formation of a product. 57 mg of a compound (hydrochloride) as a white solid was obtained through prep-HPLC purification, with a purity of 97% and a yield of 21%. HRMS[M]⁺: 1259.5494.

¹H NMR (400 MHz, CD₃OD) δ 8.45 (s, 1H), 8.12 (s, 1H), 8.02-7.95 (m, 3H), 7.65 (dd, J=8.5, 1.1 Hz, 1H), 7.33 (dd, J=11.0, 6.7 Hz, 1H), 7.15 (d, J=8.4 Hz, 2H), 7.02 (dd, J=10.7, 7.2 Hz, 1H), 6.76 (d, J=8.4 Hz, 2H), 5.42 (s, 1H), 5.05 (s, 1H), 4.81-4.75 (m, 1H), 4.60 (d, J=11.3 Hz, 3H), 4.39 (d, J=4.1 Hz, 1H), 4.33 (d, J=8.1 Hz, 2H), 4.24 (d, J=8.0 Hz, 2H), 4.21-4.16 (m, 1H), 4.09 (t, J=6.4 Hz, 3H), 4.00 (d, J=8.6 Hz, 2H), 3.96-3.88 (m, 2H), 3.82 (d, J=10.9 Hz, 1H), 3.58 (d, J=26.0 Hz, 2H), 3.51-3.46 (m, 1H), 3.13 (s, 9H), 2.55-2.41 (m, 2H), 2.30 (s, 1H), 2.09 (t, J=13.4 Hz, 2H), 1.83 (dd, J=14.3, 6.7 Hz, 2H), 1.50-1.40 (m, 4H), 1.27 (d, J=6.2 Hz, 6H), 1.08 (d, J=6.8 Hz, 3H), 0.97 (t, J=7.1 Hz, 3H).

### Example 17

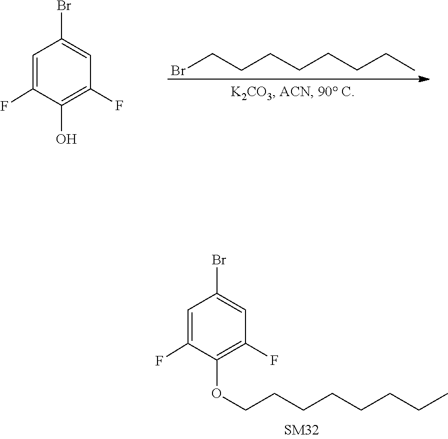

4-Bromo-2,6-difluorophenol (2 g, 9.56 mmol, 1 eq.) was dissolved in acetonitrile (45 mL), bromooctane (1.65 mL, 9.56 mmol, 1 eq.) was added, and potassium carbonate (3.969 g, 28.70 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 3.092 g of crude product SM32 as a transparent liquid, with a yield of 99%.

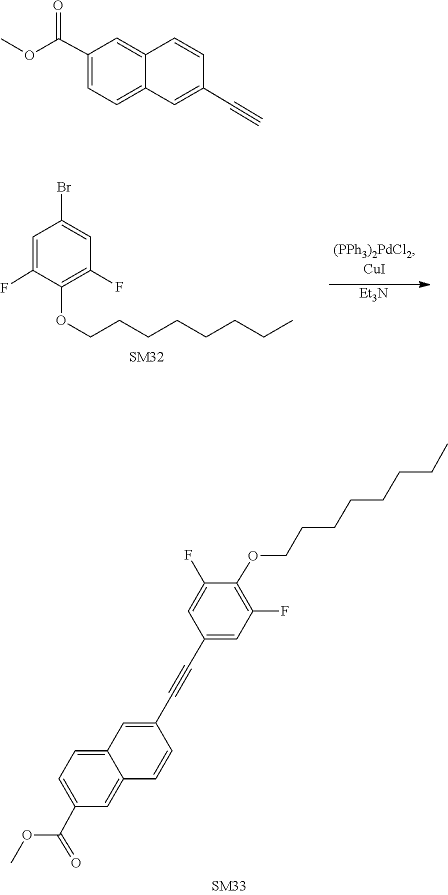

SM6 (1 g, 4.76 mmol, 1 eq.) and SM32 (1.53 g, 4.76 mmol, 1 eq.) were dissolved in triethylamine (20 mL), and (PPh₃)₂PdCl₂ (330 mg, 0.47 mmol, 0.1 eq.) and CuI (90 mg, 0.47 mmol, 0.1 eq.) were added. Replacement with nitrogen was performed. The resulting mixture was reacted at 90° C. for 2.5 h under microwave condition. TLC showed the formation of a new spot and the disappearance of the raw materials. The solvent was removed. The resulting mixture was extracted with water and DCM. The solvent was removed to obtain a crude product. The crude product was separated and purified by column chromatography to obtain 766 mg of compound SM33 as a yellow solid, with a yield of 32%.

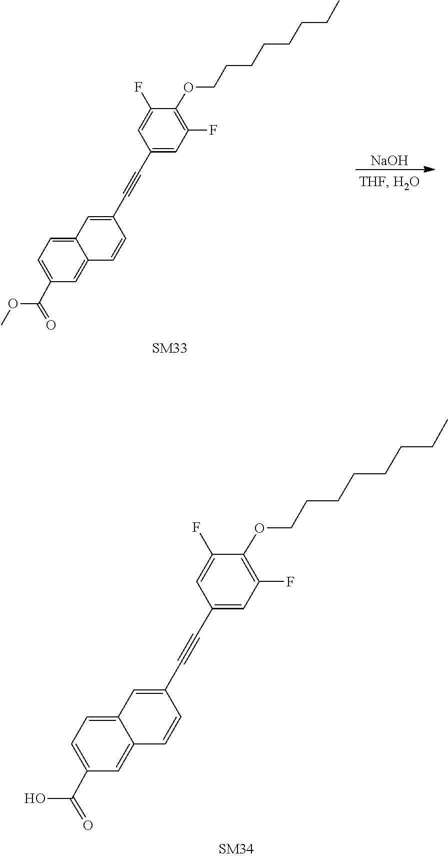 [SMILES: CCCCCCCCOc1c(F)cc(C#Cc2ccc3cc(C(=O)OC)ccc3c2)cc1F ; "NaOH" ; CCCCCCCCOc1c(F)cc(C#Cc2ccc3cc(C(=O)O)ccc3c2)cc1F]

SM33 (766 mg, 1.70 mmol, 1 eq.) was dissolved in THF (9 mL). NaOH (272 mg, 6.80 mmol, 4 eq.) was dissolved in water (1 mL) and added to the reaction system. The reaction system was stirred at 60° C. for 5 h. TLC showed a spot of product. After THF was subjected to rotary evaporation, extraction was performed with water and EA for the first time. The aqueous phase was remained for acidifying, extraction was performed with DCM, and the solvent was removed to obtain 531 mg of crude product SM34 as a yellow solid, with a yield of 71%.

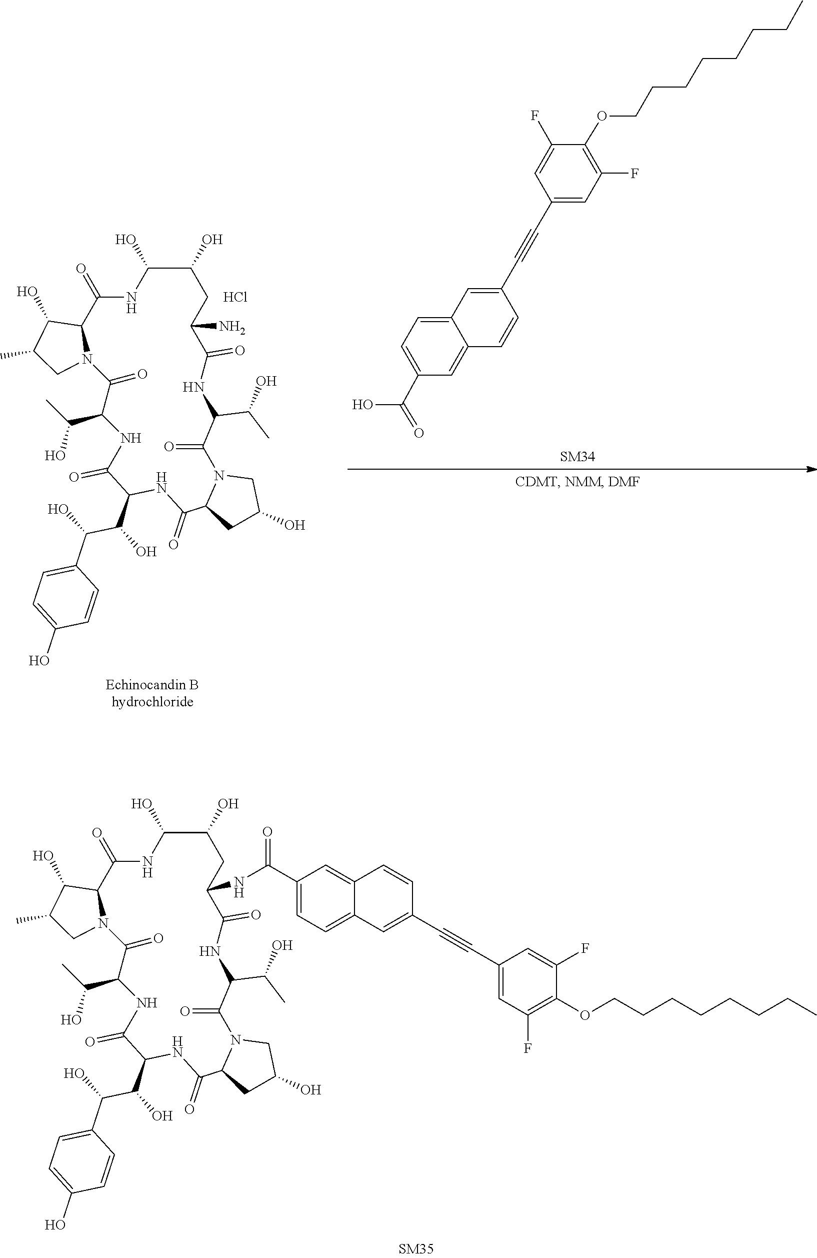

SM34 (391 mg, 0.89 mmol, 1 eq.) was dissolved in DMF (6 mL), echinocandin B hydrochloride (500 mg, 0.89 mmol, 1 eq.) and CDMT (126 mg, 1.07 mmol, 1.2 eq.) were sequentially added, and NMM (0.2 mL, 2.69 mmol, 3 eq.) was finally added. The resulting mixture was stirred for 4 h at room temperature. 409 mg of compound SM35 as a white solid was obtained through prep-HPLC purification, with a purity of 96% and a yield of 53%. HRMS[M+Na]⁺: 1238.4876.

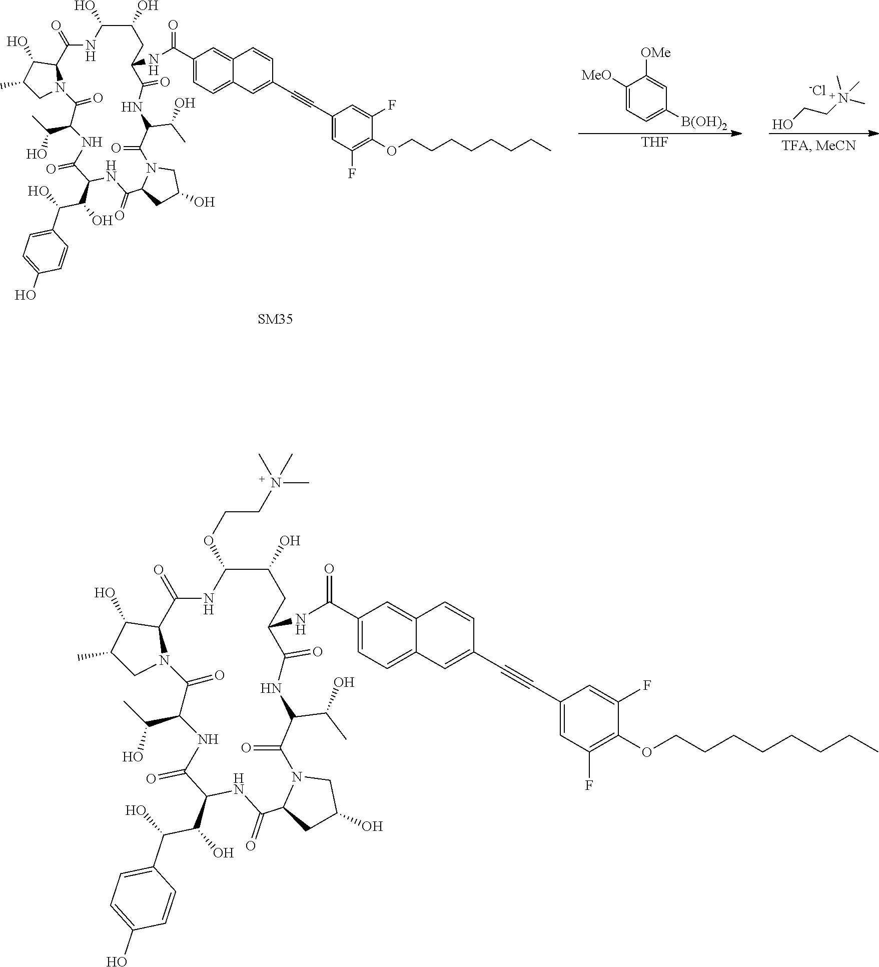

SM35 (150 mg, 0.12 mmol, 1 eq.) was dissolved in dry THF (3 mL), and 3,4-dimethoxyphenylboronic acid (29 mg, 0.16 mmol, 1.3 eq.) was added. The resulting mixture was stirred for 1 h at room temperature, the solvent was removed, and then dry THF (3 mL) was added for rotary evaporation. Choline chloride (344 mg, 1.23 mmol, 10 eq.) was added, and TFA (0.38 mL) mixed with acetonitrile (3 mL) was added to the system. The system was stirred for 3 h at room temperature. LCMS showed the formation of a product. 34 mg of a compound (hydrochloride) as a white solid was obtained through prep-HPLC purification, with a purity of 98% and a yield of 28%. HRMS[M+H]⁺: 1301.5963.

¹H NMR (400 MHz, CD₃OD) δ 8.45 (s, 1H), 8.14 (s, 1H), 8.02-7.96 (m, 3H), 7.67-7.64 (m, 1H), 7.23 (d, J=8.5 Hz, 2H), 7.15 (d, J=8.5 Hz, 2H), 6.76 (d, J=8.5 Hz, 2H), 5.42 (d, J=2.2 Hz, 1H), 5.05 (d, J=3.1 Hz, 1H), 4.78 (d, J=5.0 Hz, 1H), 4.58 (d, J=5.3 Hz, 3H), 4.39 (d, J=4.3 Hz, 1H), 4.33 (d, J=7.8 Hz, 2H), 4.25 (dd, J=9.9, 5.6 Hz, 2H), 4.19 (t, J=6.3 Hz, 3H), 4.11 (s, 1H), 4.00 (d, J=11.0 Hz, 2H), 3.94-3.88 (m, 2H), 3.82 (d, J=10.8 Hz, 1H), 3.60-3.47 (m, 3H), 3.13 (s, 9H), 2.53-2.42 (m, 2H), 2.33-2.26 (m, 1H), 2.08 (t, J=12.2 Hz, 2H), 1.79-1.73 (m, 2H), 1.50 (d, J=7.6 Hz, 2H), 1.37-1.31 (m, 8H), 1.27 (d, J=6.2 Hz, 6H), 1.08 (d, J=6.9 Hz, 3H), 0.92 (t, J=6.7 Hz, 3H).

### Example 18

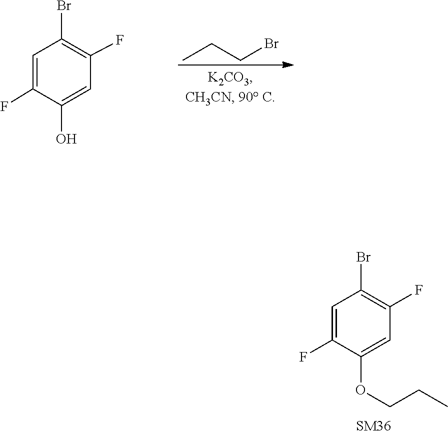

4-Bromo-2,5-difluorophenol (2 g, 9.50 mmol, 1 eq.), bromopropane (1 ml, 11.00 mmol, 1.2 eq.) and potassium carbonate (3.9 g, 28.00 mmol, 3 eq.) were dissolved in acetonitrile (45 mL), and the mixture was heated, refluxed, and stirred at 90° C. After 1.5 h, TLC showed the formation of a new spot and the disappearance of the raw materials. The solvent was removed, ethyl acetate and water were added for extraction, the organic phases were combined, dried with anhydrous sodium sulfate, and concentrated to obtain 2.2 g of compound SM36 as a yellow oil, with a yield of 95%.

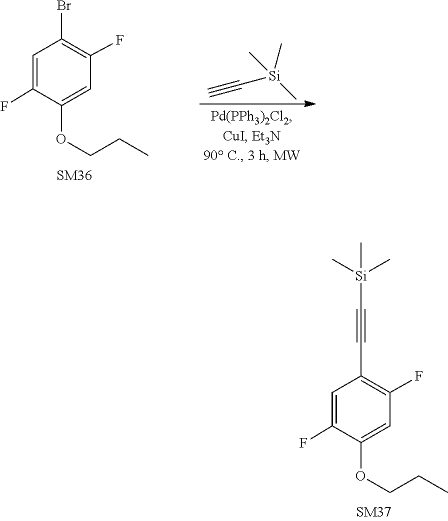

SM36 (1 g, 4.00 mmol, 1 eq.), trimethylsilylacetylene (393 mg, 4.00 mmol, 1 eq.), Pd(PPh₃)₂Cl₂ (280 mg, 0.40 mmol, 0.1 eq.) and CuI (70 mg, 0.40 mmol, 0.1 eq.) were weighed and placed in a microwave reaction bottle. Triethylamine (15 mL) was added. Nitrogen was blown into the bottle by using a bubbling method to vent the air. After 15 min, the bottle was quickly covered for sealing, and the mixture was reacted under microwave condition for 3 h at 90° C. After the reaction was completed, there was no raw material left in the TLC dot plate. The solvent was removed, DCM was added for dissolving, and 690 mg of compound SM37 as a yellow oil was obtained by column chromatography, with a yield of 64%.

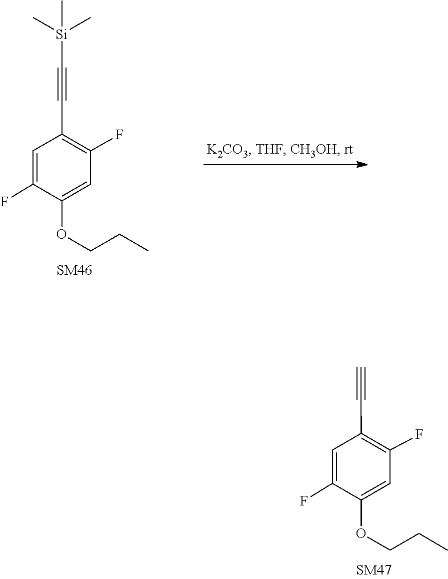 [SMILES: CCCOc1cc(F)c(C#C[Si](C)(C)C)cc1F ; C#Cc1cc(F)c(OCCC)cc1F]

SM37 (690 mg, 2.50 mmol, 1 eq.) and potassium carbonate (535 mg, 3.80 mmol, 1.5 eq.) were dissolved in THF (5 mL) and CH₃OH (5 mL). The resulting mixture was stirred overnight at room temperature. TLC showed that the reaction was finished. The solvent was removed. Water and EA were added for extraction. The organic phase was dried and concentrated to obtain 471 mg of SM38 as a yellow oil, with a yield of 93%.

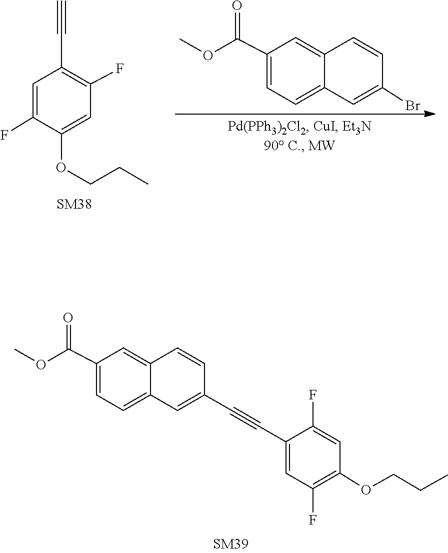 [SMILES: C#Cc1cc(F)c(OCCC)cc1F ; COC(=O)c1ccc2cc(Br)ccc2c1 ; CCCOc1cc(F)c(C#Cc2ccc3cc(C(=O)OC)ccc3c2)cc1F]

SM38 (471 mg, 2.40 mmol, 1.2 eq), methyl 6-bromo-2-naphthoate (533 mg, 2.00 mmol, 1 eq.), Pd(PPh₃)₂Cl₂ (141 mg, 0.20 mmol, 0.1 eq.) and CuI (38 mg, 0.20 mmol, 0.1 eq.) were weighed and placed in a microwave reaction bottle. Triethylamine (15 mL) was added. Nitrogen was blown into the bottle by using a bubbling method to vent the air. After about 15 min, the bottle was quickly covered for sealing, and the mixture was reacted under microwave condition for 3 h at 90° C. After the reaction was completed, there was no raw material left in the TLC dot plate. The solvent was removed, DCM was added for dissolving, and 555 mg of compound SM39 as a yellow solid was obtained by column chromatography, with a yield of 72%.

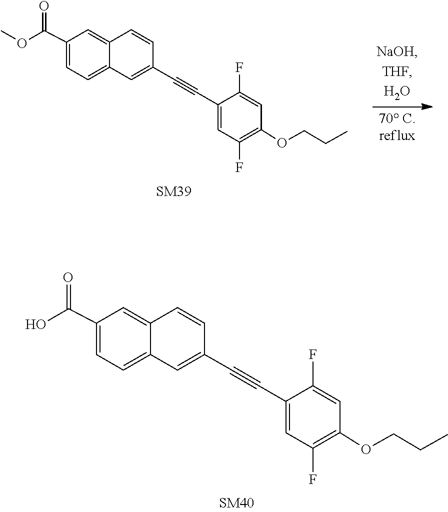 [SMILES: CCCOc1cc(F)c(C#Cc2ccc3cc(C(=O)OC)ccc3c2)cc1F ; CCCOc1cc(F)c(C#Cc2ccc3cc(C(=O)O)ccc3c2)cc1F]

SM39 (555 mg, 1.40 mmol, 1 eq.) was dissolved in THF (5 mL), and heated and stirred at 70° C., and then an aqueous solution (1 mL) of NaOH (234 mg, 5.80 mmol, 4 eq.) was added to the reaction system. The reaction system was heated at 70° C. and refluxed overnight. After the reaction was completed, 2M HCl (aq.) was added to adjust the pH value until an acidic state is reached, and then a solid was precipitated. Suction filtration was performed to obtain 457 mg of compound SM40 as a white solid, with a yield of 85%.

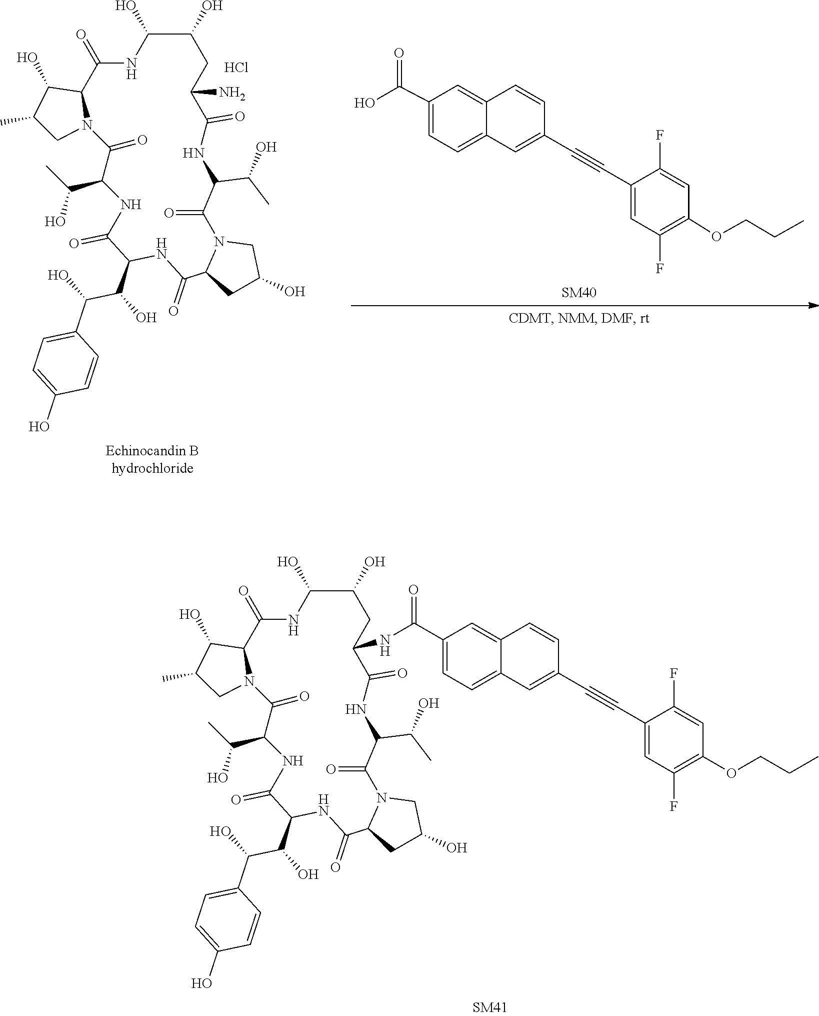

Echinocandin B hydrochloride (300 mg, 0.35 mmol, 1 eq.), SM40 (131 mg, 0.35 mmol, 1 eq.) and CDMT (75 mg, 0.43 mmol, 1.2 eq.) were dissolved in DMF (3.5 mL), and then NMM (0.118 mL, 1.07 mmol, 3 eq.) was added. Stirring was performed for 1 h at room temperature. After the reaction was completed, the reaction solution was subjected to prep-HPLC purification and separation to obtain 235 mg of compound SM41 as a white solid, with a purity of 86% and a yield of 57%. MS[M+H]⁺: 1146.

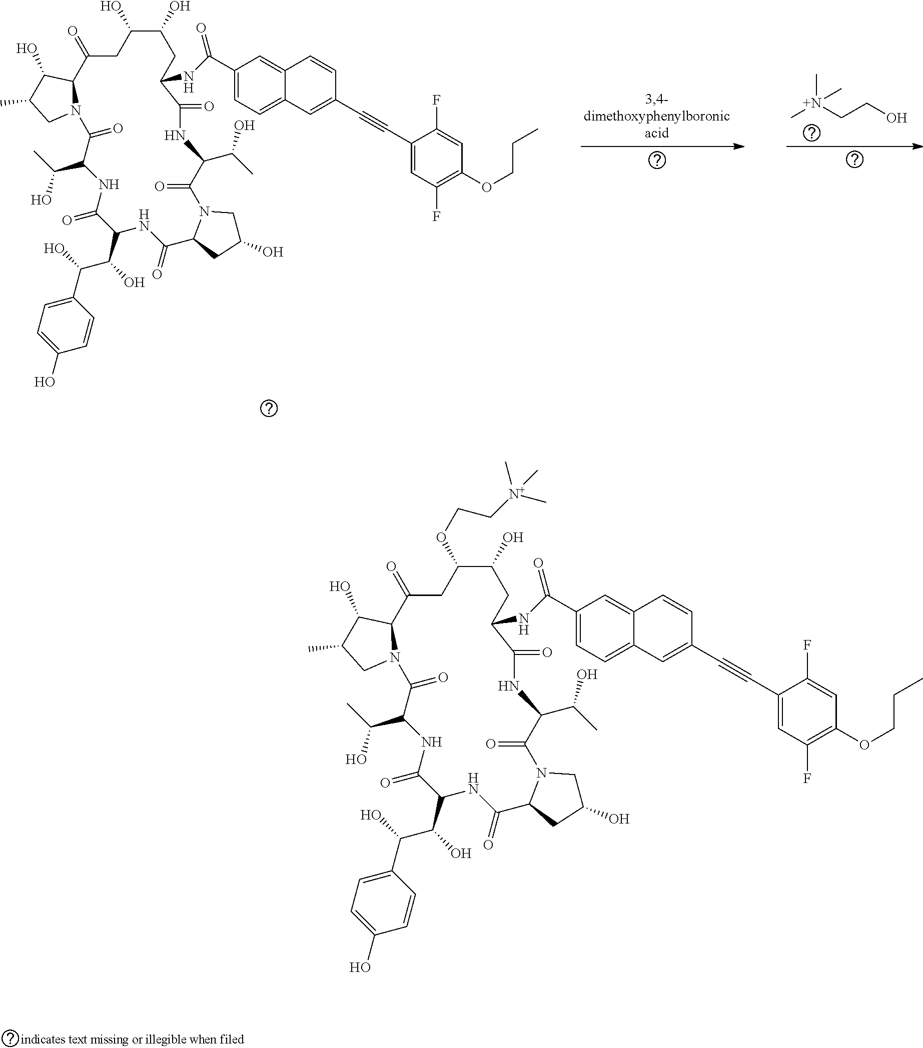

SM41 (235 mg, 0.20 mmol, 1 eq.) and 3,4-dimethoxyphenylboronic acid (49 mg, 0.26 mmol, 1.3 eq.) were dissolved in dry THF (3 mL). The resulting solution was stirred for 1 h at room temperature, and concentrated to dryness. Choline tosylate (1.7 g, 6.10 mmol, 30 eq.) was added, then a mixed solution of TFA (0.5 mL) and acetonitrile (3 mL) was added to dissolve the reactants, and stirring was performed for 5 h at room temperature under N₂ atmosphere. After the reaction was completed, an aqueous solution of sodium acetate was added for quenching, and 160 mg of a compound (hydrochloride) as a white solid was obtained through prep-HPLC purification, with a purity of 96% and a yield of 63%. HRMS[M]⁺: 1231.5424.

¹H NMR (400 MHz, CD₃OD) δ 8.45 (s, 1H), 8.13 (s, 1H), 8.02-7.96 (m, 3H), 7.65 (d, J=8.4 Hz, 1H), 7.33 (dd, J=11.0, 6.7 Hz, 1H), 7.15 (d, J=8.4 Hz, 2H), 7.03 (dd, J=10.7, 7.2 Hz, 1H), 6.76 (d, J=8.5 Hz, 2H), 5.42 (s, 1H), 5.05 (d, J=2.9 Hz, 1H), 4.79 (dd, J=12.0, 5.0 Hz, 1H), 4.60 (d, J=11.5 Hz, 3H), 4.39 (d, J=4.2 Hz, 1H), 4.33 (d, J=8.1 Hz, 2H), 4.25 (t, J=7.5 Hz, 2H), 4.21-4.16 (m, 1H), 4.11 (s, 1H), 4.06 (t, J=6.4 Hz, 3H), 4.00 (d, J=11.0 Hz, 1H), 3.95-3.88 (m, 2H), 3.83 (d, J=11.0 Hz, 1H), 3.55-3.47 (m, 2H), 3.13 (s, 9H), 2.55-2.42 (m, 2H), 2.30 (dd, J=11.5, 6.1 Hz, 1H), 2.08 (dd, J=15.1, 10.5 Hz, 2H), 1.86 (dd, J=14.0, 6.7 Hz, 2H), 1.27 (d, J=6.2 Hz, 6H), 1.10-1.05 (m, 6H).

### Example 19

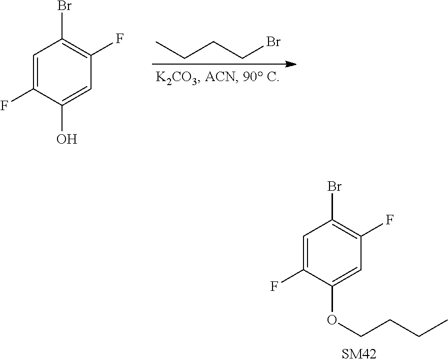

4-Bromo-2,5-difluorophenol (2 g, 9.50 mmol, 1 eq.), bromobutane (1 ml, 11.00 mmol, 1.2 eq.) and potassium carbonate (3.9 g, 28.00 mmol, 3 eq.) were dissolved in acetonitrile (45 mL), and the mixture was heated, refluxed, and stirred at 90° C. After 1.5 h, TLC showed the formation of a new spot and the disappearance of the raw materials. The solvent was removed, ethyl acetate and water were added for extraction, the organic phases were combined, dried with anhydrous sodium sulfate, and concentrated to obtain 2.28 g of compound SM51 as a yellow oil, with a yield of 90%.

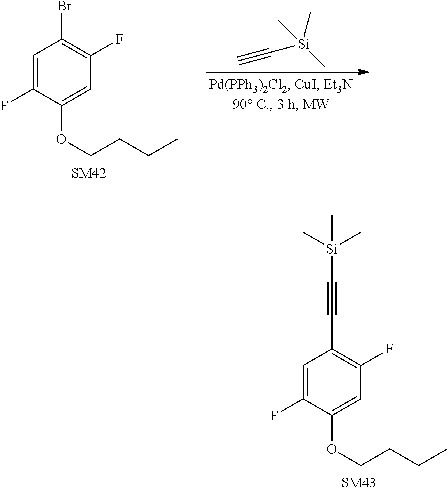

SM42 (1 g, 3.70 mmol, 1 eq.), trimethylsilylacetylene (446 mg, 4.50 mmol, 1.2 eq.), Pd(PPh₃)₂Cl₂ (265 mg, 0.37 mmol, 0.1 eq.) and CuI (72 mg, 0.37 mmol, 0.1 eq.) were weighed and placed in a microwave reaction bottle. Triethylamine (15 mL) was added. Nitrogen was blown into the bottle by using a bubbling method to vent the air. After 15 min, the bottle was quickly covered for sealing, and the mixture was reacted under microwave condition for 3 h at 90° C. After the reaction was completed, there was no raw material left in the TLC dot plate. The solvent was removed, DCM was added for dissolving, and 992 mg of compound SM43 as a yellow oil was obtained by column chromatography, with a yield of 93%.

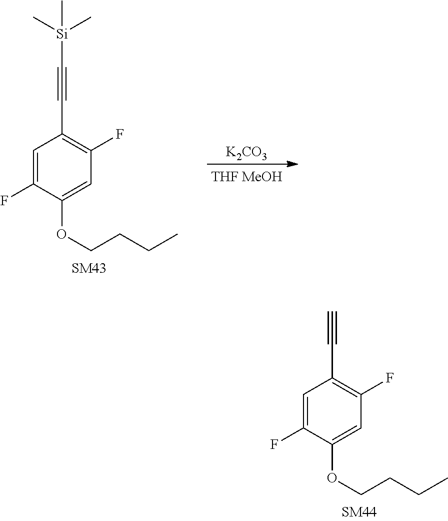 [SMILES: CCCCOc1cc(F)c(C#C[Si](C)(C)C)cc1F ; "K2CO3" ; C#Cc1cc(F)c(OCCCC)cc1F]

SM43 (992 mg, 3.50 mmol, 1 eq.) and potassium carbonate (731 mg, 5.20 mmol, 1.5 eq.) were dissolved in THF (5 mL) and CH₃OH (5 mL). The resulting mixture was stirred overnight at room temperature. TLC showed that the reaction was finished. The solvent was removed. Water and EA were added for extraction. The organic phase was dried and concentrated to obtain 672 mg of SM44 as a yellow oil, with a yield of 91%.

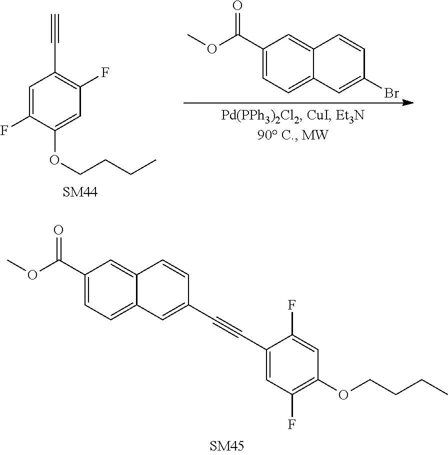 [SMILES: C#Cc1cc(F)c(OCCCC)cc1F ; COC(=O)c1ccc2cc(Br)ccc2c1 ; CCCCOc1cc(F)c(C#Cc2ccc3cc(C(=O)OC)ccc3c2)cc1F]

SM44 (672 mg, 3.20 mmol, 1.5 eq), methyl 6-bromo-2-naphthoate (568 mg, 2.10 mmol, 1 eq.), Pd(PPh₃)₂Cl₂ (150 mg, 0.20 mmol, 0.1 eq.) and CuI (40 mg, 0.20 mmol, 0.1 eq.) were weighed and placed in a microwave reaction bottle. Triethylamine (15 mL) was added. Nitrogen was blown into the bottle by using a bubbling method to vent the air. After 15 min, the bottle was quickly covered for sealing, and the mixture was reacted under microwave condition for 3 h at 90° C. After the reaction was completed, there was no raw material left in the TLC dot plate. The solvent was removed, DCM was added for dissolving, and 635 mg of compound SM45 as a yellow solid was obtained by column chromatography, with a yield of 75%.

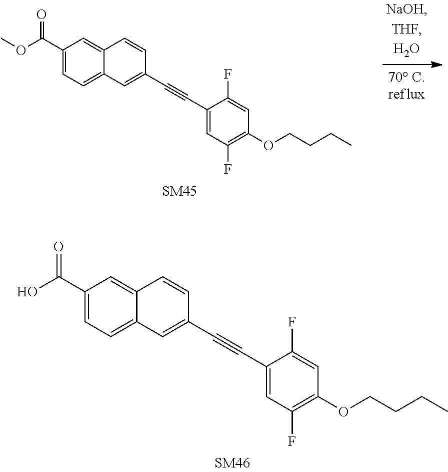 [SMILES: CCCCOc1cc(F)c(C#Cc2ccc3cc(C(=O)OC)ccc3c2)cc1F ; CCCCOc1cc(F)c(C#Cc2ccc3cc(C(=O)O)ccc3c2)cc1F]

SM45 (635 mg, 1.60 mmol, 1 eq.) was dissolved in THF (7 mL), and heated and stirred at 70° C., and then an aqueous solution (1.5 mL) of NaOH (258 mg, 6.40 mmol, 4 eq.) was added to the reaction system. The reaction system was heated at 70° C. and refluxed overnight. After the reaction was completed, 2M HCl (aq.) was added to adjust the pH value until an acidic state is reached, and then a solid was precipitated. Suction filtration was performed to obtain 586 mg of compound SM46 as a yellow solid, with a yield of 95%.

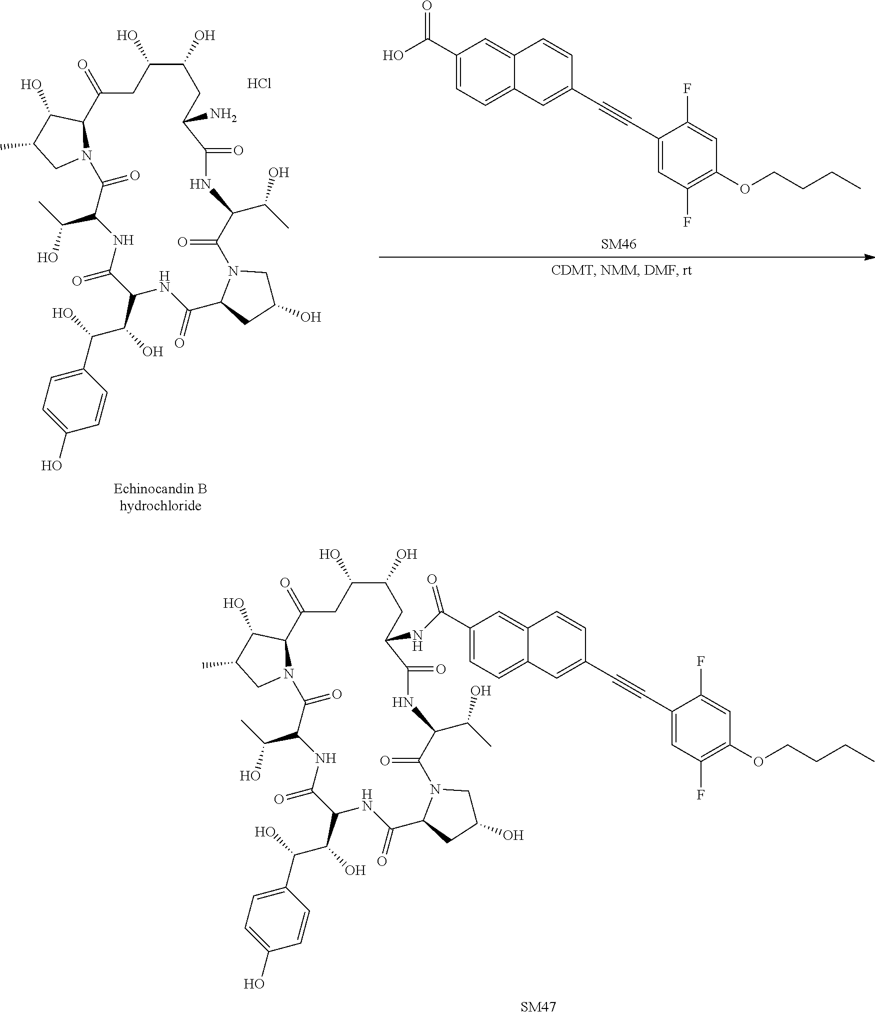

Echinocandin B hydrochloride (300 mg, 0.35 mmol, 1 eq.), SM46 (136 mg, 0.35 mmol, 1 eq.) and CDMT (75 mg, 0.43 mmol, 1.2 eq.) were dissolved in DMF (3.5 mL), and then NMM (0.118 mL, 1.07 mmol, 3 eq.) was added. Stirring was performed for 1 h at room temperature. After the reaction was completed, the reaction solution was subjected to prep-HPLC purification and separation to obtain 252 mg of compound SM47 as a white solid, with a purity of 99% and a yield of 60%. MS[M+H]⁺: 1161.

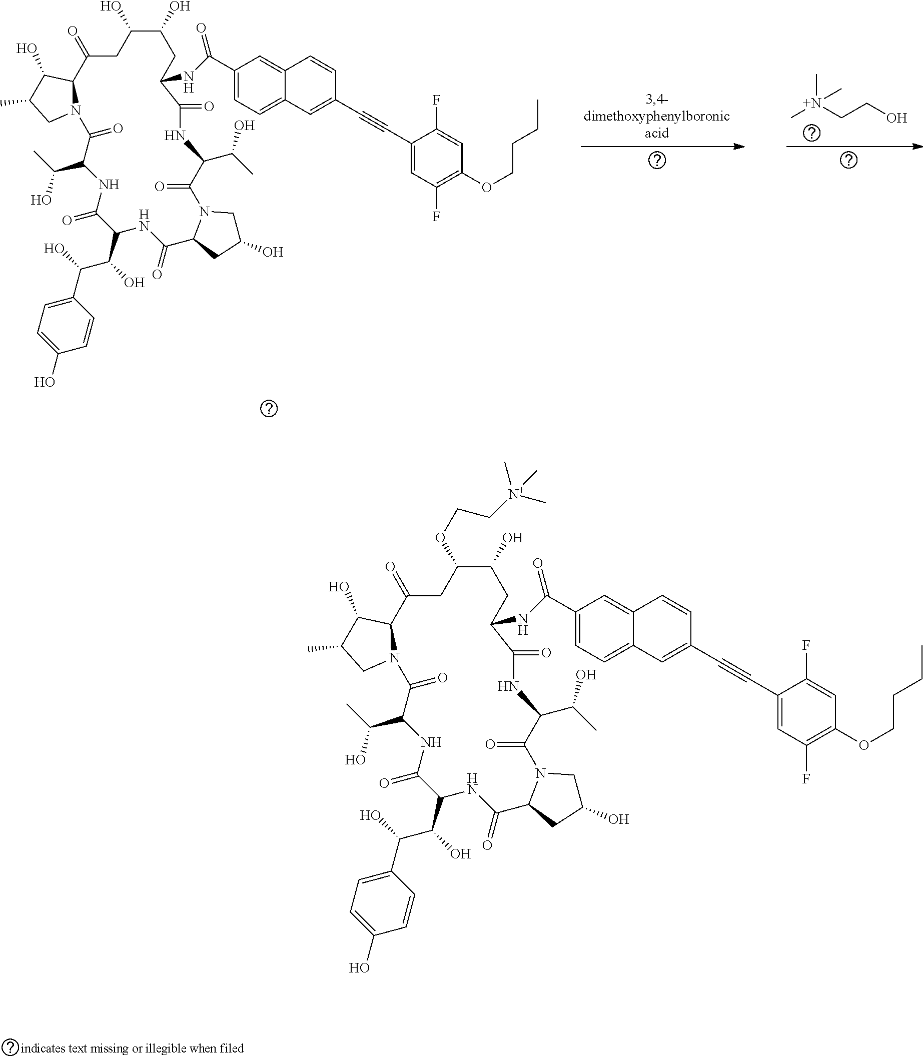

SM47 (252 mg, 0.20 mmol, 1 eq.) and 3,4-dimethoxyphenylboronic acid (51 mg, 0.28 mmol, 1.3 eq.) were dissolved in dry THF (3 mL). The resulting solution was stirred for 1 h at room temperature, and concentrated to dryness. Choline tosylate (1.79 g, 6.50 mmol, 30 eq.) was added, then a mixed solution of TFA (0.5 mL) and acetonitrile (3 mL) was added to dissolve the reactants, and stirring was performed for 5 h at room temperature under N₂ atmosphere. After the reaction was completed, an aqueous solution of sodium acetate was added for quenching, and 93 mg of a compound (hydrochloride) as a white solid was obtained through prep-HPLC purification, with a purity of 96% and a yield of 34%. HRMS [M]⁺: 1245.5582.

¹H NMR (400 MHz, CD₃OD) δ 8.45 (s, 1H), 8.13 (s, 1H), 8.03-7.97 (m, 3H), 7.65 (dd, J=8.5, 1.4 Hz, 1H), 7.33 (dd, J=11.1, 6.7 Hz, 1H), 7.15 (d, J=8.5 Hz, 2H), 7.03 (dd, J=10.7, 7.2 Hz, 1H), 6.76 (d, J=8.5 Hz, 2H), 5.42 (d, J=2.5 Hz, 1H), 5.04 (d, J=3.2 Hz, 1H), 4.79 (dd, J=12.0, 5.1 Hz, 1H), 4.59 (dd, J=10.7, 7.2 Hz, 4H), 4.39 (d, J=4.3 Hz, 1H), 4.33 (d, J=8.3 Hz, 2H), 4.25 (dd, J=8.2, 6.4 Hz, 2H), 4.20-4.16 (m, 1H), 4.10 (t, J=6.4 Hz, 3H), 4.00 (d, J=11.2 Hz, 1H), 3.95-3.88 (m, 2H), 3.83 (d, J=10.5 Hz, 1H), 3.60 (s, 1H), 3.49 (d, J=9.6 Hz, 2H), 3.13 (s, 9H), 2.53-2.42 (m, 2H), 2.30 (s, 1H), 2.10 (d, J=12.1 Hz, 2H), 1.85-1.79 (m, 2H), 1.54 (dd, J=15.1, 7.5 Hz, 2H), 1.27 (d, J=6.3 Hz, 6H), 1.08 (d, J=6.9 Hz, 3H), 1.01 (t, J=7.4 Hz, 3H).

### Example 20

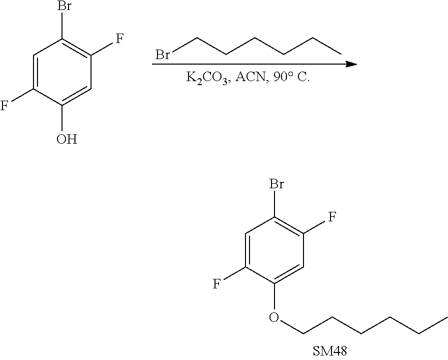

4-Bromo-2,5-difluorophenol (2 g, 9.56 mmol, 1 eq.) was dissolved in acetonitrile (45 mL), bromohexane (2.01 mL, 14.35 mmol, 1.5 eq.) was added, and potassium carbonate (3.969 g, 28.70 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 2.551 g of crude product SM48 as a transparent liquid, with a yield of 91%.

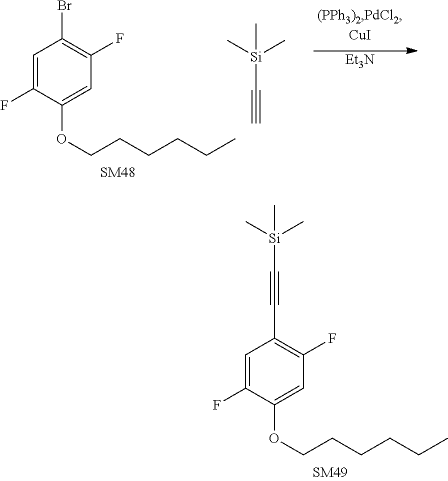

SM48 (1 g, 3.41 mmol, 1 eq.) was dissolved in triethylamine (17 mL), and trimethylsilylacetylene (0.48 mL, 3.41 mmol, 1 eq.), Pd(PPh₃)₂Cl₂ (239 mg, 0.34 mmol, 0.1 eq.) and CuI (65 mg, 0.34 mmol, 0.1 eq.) were added. The resulting mixture was reacted at 90° C. for 3 h under microwave condition. TLC showed the completion of the reaction and the formation of a new spot. Column chromatography separation and purification was performed to obtain 851 mg of compound SM49 as a light yellow liquid, with a yield of 80%.

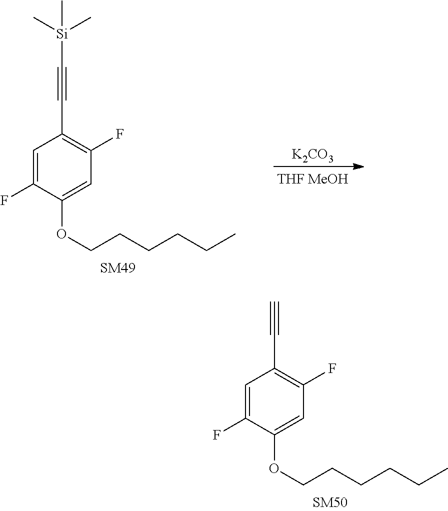 [SMILES: CCCCCCOc1cc(F)c(C#C[Si](C)(C)C)cc1F ; "K2CO3" ; C#Cc1cc(F)c(OCCCCCC)cc1F]

SM49 (851 mg, 2.74 mmol, 1 eq.) was dissolved in MeOH (13 mL) and THF (13 mL). Potassium carbonate (569 mg, 4.11 mmol, 1.5 eq.) was added. The resulting mixture was stirred for 3 h at room temperature. TLC showed the completion of the reaction, the formation of a new spot, and the disappearance of the raw materials. The resulting mixture was extracted with water and EA. The EA phase was subjected to rotary evaporation to obtain 578 mg of SM50 as an oily liquid, with a yield of 88%.

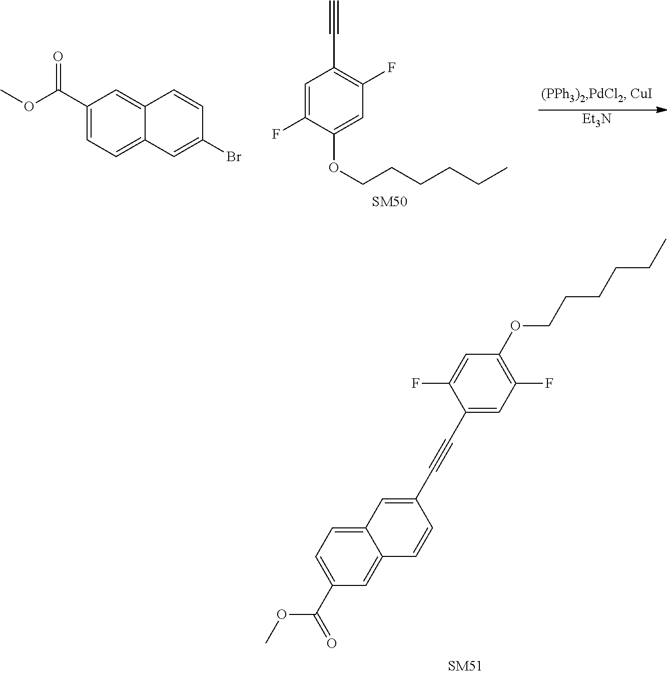 [SMILES: "Et3N" ; COC(=O)c1ccc2cc(Br)ccc2c1 ; C#Cc1cc(F)c(OCCCCCC)cc1F ; CCCCCCOc1cc(F)c(C#Cc2ccc3cc(C(=O)OC)ccc3c2)cc1F]

Methyl 6-bromo-2-naphthoate (644 mg, 2.42 mmol, 1 eq.) was dissolved in triethylamine (12 mL), and SM50 (578 mg, 2.42 mmol, 1 eq.), (PPh₃)₂PdCl₂ (170 mg, 0.24 mmol, 0.1 eq.) and CuI (46 mg, 0.24 mmol, 0.1 eq.) were added. Replacement with nitrogen was performed. The resulting mixture was stirred for 3 h at 90° C. under microwave condition. TLC showed the formation of a new spot and the disappearance of the raw materials. Column chromatography separation and purification was performed to obtain 854 mg of compound SM51 as a yellow solid, with a yield of 83%.

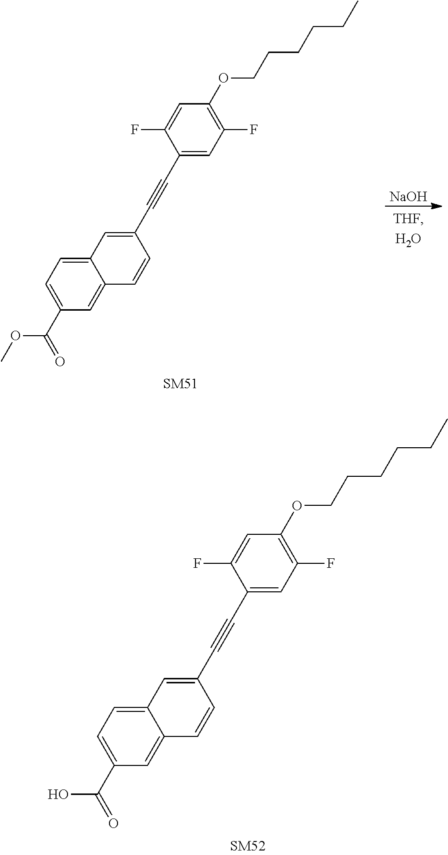 [SMILES: CCCCCCOc1cc(F)c(C#Cc2ccc3cc(C(=O)OC)ccc3c2)cc1F ; "NaOH" ; CCCCCCOc1cc(F)c(C#Cc2ccc3cc(C(=O)O)ccc3c2)cc1F]

SM51 (854 mg, 2.02 mmol, 1 eq.) was dissolved in THF (10 mL). NaOH (324 mg, 8.09 mmol, 4 eq.) was dissolved in water (1 mL) and added to the reaction system. The reaction system was stirred overnight at 60° C. TLC showed a spot of product. Acidifying was performed, and a solid was precipitated. Suction filtration and drying were performed to obtain 698 mg of compound SM52 as a yellow solid, with a yield of 84%.

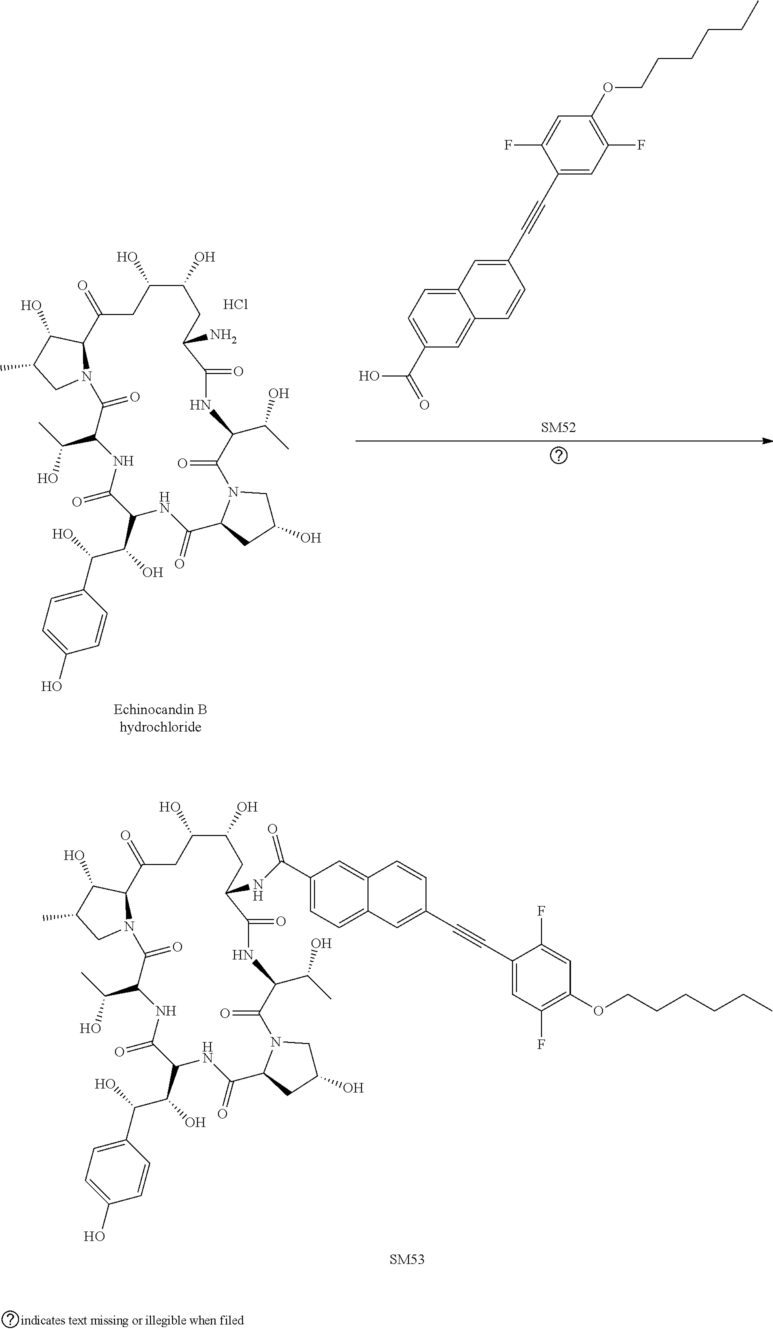

SM52 (300 mg, 0.73 mmol, 1 eq.) was dissolved in DMF (7 mL), echinocandin B hydrochloride (613 mg, 0.73 mmol, 1 eq.) and CDMT (155 mg, 0.88 mmol, 1.2 eq.) were sequentially added, and NMM (0.24 mL, 2.20 mmol, 3 eq.) was finally added. The resulting mixture was stirred for 4 h at room temperature. 435 mg of compound SM53 as a white solid was obtained through prep-HPLC purification, with a purity of 91% and a yield of 50%. HRMS [M+H]⁺: 1210.4887.

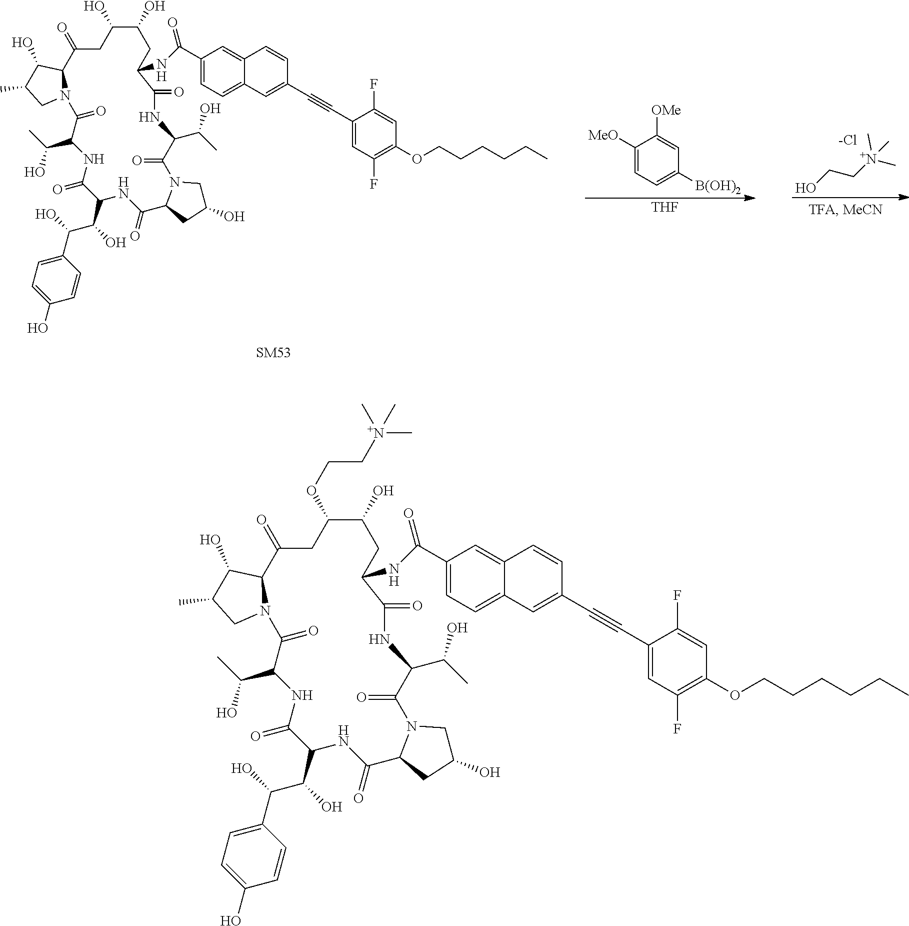

SM53 (335 mg, 0.28 mmol, 1 eq.) was dissolved in dry THF (7 mL), and 3,4-dimethoxyphenylboronic acid (67 mg, 0.36 mmol, 1.3 eq.) was added. The resulting mixture was stirred for 1 h at room temperature, the solvent was removed, and then dry THF (7 mL) was added for rotary evaporation. Choline chloride (394 mg, 2.82 mmol, 10 eq.) was added, and TFA (0.84 mL) mixed with acetonitrile (7 mL) was added to the system. The system was stirred for 3 h at room temperature. LCMS showed the formation of a product. 9 mg of a compound (hydrochloride) as a white solid was obtained through prep-HPLC purification, with a purity of 99% and a yield of 2%. HRMS[M]⁺: 1273.5882.

¹H NMR (400 MHz, CD₃OD) δ 8.45 (s, 1H), 8.12 (s, 1H), 8.01-7.96 (m, 3H), 7.70 (d, J=8.2 Hz, 1H), 7.33 (dd, J=11.0, 6.7 Hz, 1H), 7.15 (d, J=8.6 Hz, 2H), 7.02 (dd, J=10.8, 7.2 Hz, 1H), 6.76 (d, J=8.6 Hz, 2H), 5.42 (d, J=2.4 Hz, 1H), 5.05 (d, J=3.2 Hz, 1H), 4.79 (dd, J=12.1, 5.1 Hz, 1H), 4.58 (d, J=6.4 Hz, 3H), 4.39 (d, J=4.3 Hz, 1H), 4.33 (d, J=8.0 Hz, 2H), 4.27-4.22 (m, 2H), 4.20-4.16 (m, 1H), 4.09 (t, J=6.4 Hz, 3H), 4.00 (d, J=11.2 Hz, 1H), 3.94-3.87 (m, 2H), 3.82 (d, J=10.9 Hz, 1H), 3.64-3.46 (m, 4H), 3.13 (s, 9H), 2.53-2.42 (m, 2H), 2.34-2.27 (m, 1H), 2.08 (t, J=13.4 Hz, 2H), 1.84 (dd, J=14.3, 7.3 Hz, 2H), 1.54-1.48 (m, 2H), 1.38 (dd, J=7.2, 3.5 Hz, 4H), 1.27 (d, J=6.3 Hz, 6H), 1.07 (d, J=6.9 Hz, 3H), 0.94 (t, J=7.0 Hz, 3H).

### Example 21

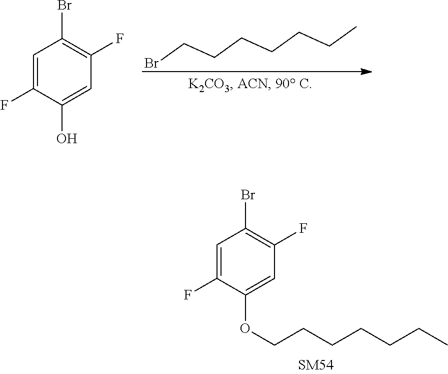

4-Bromo-2,5-difluorophenol (2 g, 9.56 mmol, 1 eq.) was dissolved in acetonitrile (45 mL), bromoheptane (2.25 mL, 14.35 mmol, 1.5 eq.) was added, and potassium carbonate (3.969 g, 28.70 mmol, 3 eq.) was finally added. The mixture was heated to 90° C., and refluxed and stirred for 4 h. TLC showed that the reaction was finished. After suction filtration, the filtrate was subjected to rotary evaporation, and extracted with water and EA. The EA phase was subjected to rotary evaporation to obtain 2.905 g of crude product SM54 as a transparent liquid, with a yield of 97%.

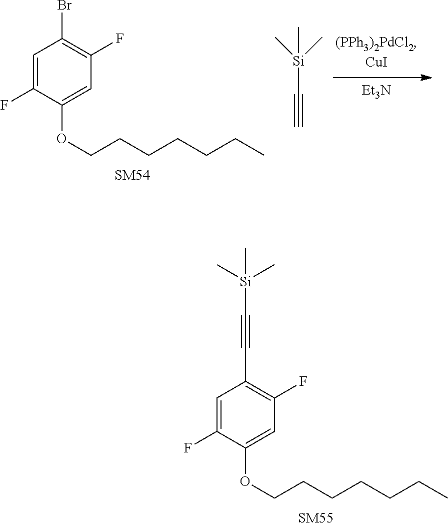

SM54 (1 g, 3.25 mmol, 1 eq.) was dissolved in triethylamine (15 mL), and trimethylsilylacetylene (0.46 mL, 3.25 mmol, 1 eq.), Pd(PPh₃)₂Cl₂ (229 mg, 0.32 mmol, 0.1 eq.) and CuI (62 mg, 0.32 mmol, 0.1 eq.) were added. The resulting mixture was reacted at 90° C. for 3 h under microwave condition. TLC showed the completion of the reaction and the formation of a new spot. Column chromatography separation and purification was performed to obtain 740 mg of compound SM55 as a light yellow liquid, with a yield of 70%.

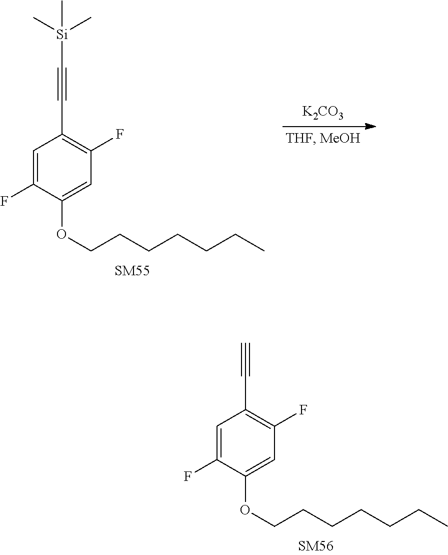 [SMILES: CCCCCCCOc1cc(F)c(C#C[Si](C)(C)C)cc1F ; C#Cc1cc(F)c(OCCCCCCC)cc1F]

SM55 (740 mg, 2.28 mmol, 1 eq.) was dissolved in MeOH (11 mL) and THF (11 mL). Potassium carbonate (473 mg, 3.42 mmol, 1.5 eq.) was added. The resulting mixture was stirred for 3 h at room temperature. TLC showed the completion of the reaction. The resulting mixture was extracted with water and EA. The EA phase was subjected to rotary evaporation to obtain 495 mg of compound SM56 as a yellow oil, with a yield of 86%.

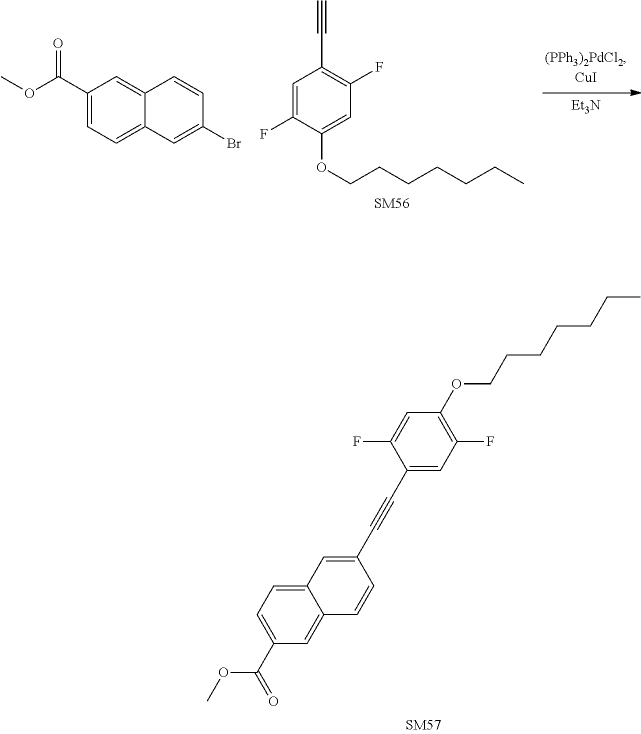

Methyl 6-bromo-2-naphthoate (521 mg, 1.96 mmol, 1 eq.) was dissolved in triethylamine (15 mL), and SM56 (495 mg, 1.96 mmol, 1 eq.), (PPh₃)₂PdCl₂ (138 mg, 0.19 mmol, 0.1 eq.) and CuI (37 mg, 0.19 mmol, 0.1 eq.) were added. Replacement with nitrogen was performed. The resulting mixture was stirred for 3 h at 90° C. under microwave. TLC showed the formation of a new spot and the disappearance of the raw materials. Column chromatography separation and purification was performed to obtain 584 mg of compound SM57 as a yellow solid, with a yield of 68%.

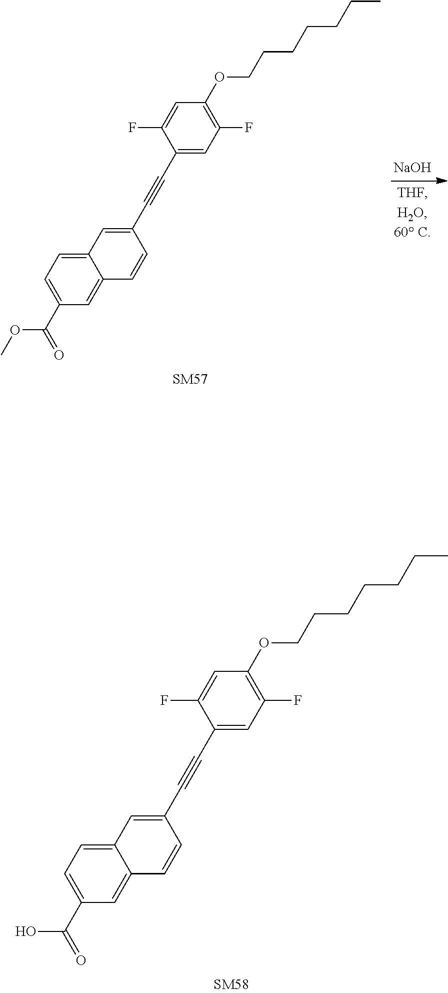 [SMILES: CCCCCCCOc1cc(F)c(C#Cc2ccc3cc(C(=O)OC)ccc3c2)cc1F ; CCCCCCCOc1cc(F)c(C#Cc2ccc3cc(C(=O)O)ccc3c2)cc1F]

SM57 (584 mg, 2.02 mmol, 1 eq.) was dissolved in THF (5 mL). NaOH (214 mg, 8.09 mmol, 4 eq.) was dissolved in water (1 mL) and added to the reaction system. The reaction system was stirred for 3 h at 60° C. TLC showed the completion of the reaction. Acidifying was performed, and then suction filtration and drying were performed to obtain 683 mg of compound SM58 as a yellow solid, with a yield of 80%.

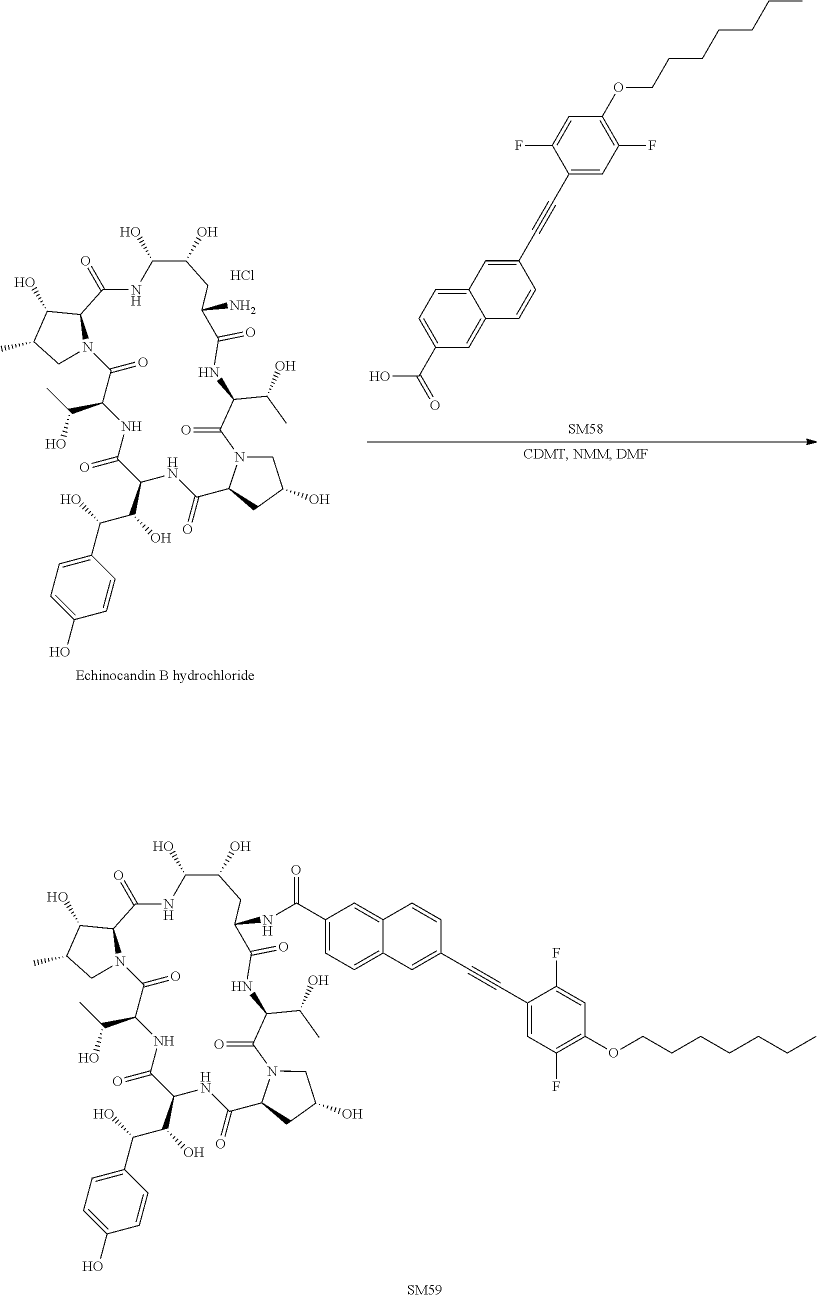 [SMILES: C[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](N)C[C@@H](O)[C@@H](O)NC(=O)[C@@H]2[C@@H](O)[C@@H](C)CN2C1=O ; Cl ; CCCCCCCOc1cc(F)c(C#Cc2ccc3cc(C(=O)O)ccc3c2)cc1F ; CCCCCCCOc1cc(F)c(C#Cc2ccc3cc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)ccc3c2)cc1F]

SM58 (300 mg, 0.73 mmol, 1 eq.) was dissolved in DMF (7 mL), echinocandin B hydrochloride (593 mg, 0.73 mmol, 1 eq.) and CDMT (150 mg, 0.88 mmol, 1.2 eq.) were sequentially added, and NMM (0.23 mL, 2.20 mmol, 3 eq.) was finally added. The resulting mixture was stirred for 4 h at room temperature. 144 mg of compound SM59 as a white solid was obtained through prep-HPLC purification, with a purity of 91% and a yield of 16%. HRMS [M+Na]⁺: 1224.4853.

¹H NMR (400 MHz, CD₃OD) δ 8.41 (s, 1H), 8.09 (s, 1H), 7.94 (dd, J=17.0, 5.8 Hz, 3H), 7.62 (d, J=9.9 Hz, 1H), 7.33 (dd, J=11.0, 6.7 Hz, 1H), 7.15 (d, J=8.5 Hz, 2H), 7.02 (dd, J=10.7, 7.2 Hz, 1H), 6.76 (d, J=8.5 Hz, 2H), 5.39-5.34 (m, 1H), 5.03 (d, J=5.6 Hz, 1H), 4.84 (s, 1H), 4.70 (dd, J=11.9, 5.2 Hz, 1H), 4.64-4.53 (m, 3H), 4.34 (dd, J=18.3, 5.2 Hz, 3H), 4.23 (dd, J=12.7, 7.0 Hz, 3H), 4.09 (t, J=6.4 Hz, 3H), 3.99 (d, J=8.1 Hz, 1H), 3.93-3.80 (m, 2H), 3.41 (t, J=9.2 Hz, 1H), 2.58-2.40 (m, 2H), 2.28-2.20 (m, 1H), 2.10 (ddd, J=25.0, 16.8, 8.2 Hz, 2H), 1.87-1.79 (m, 2H), 1.50 (dd, J=15.2, 7.4 Hz, 2H), 1.43-1.32 (m, 6H), 1.28 (dd, J=12.2, 6.2 Hz, 6H), 1.06 (d, J=6.9 Hz, 3H), 0.93 (t, J=6.8 Hz, 3H).

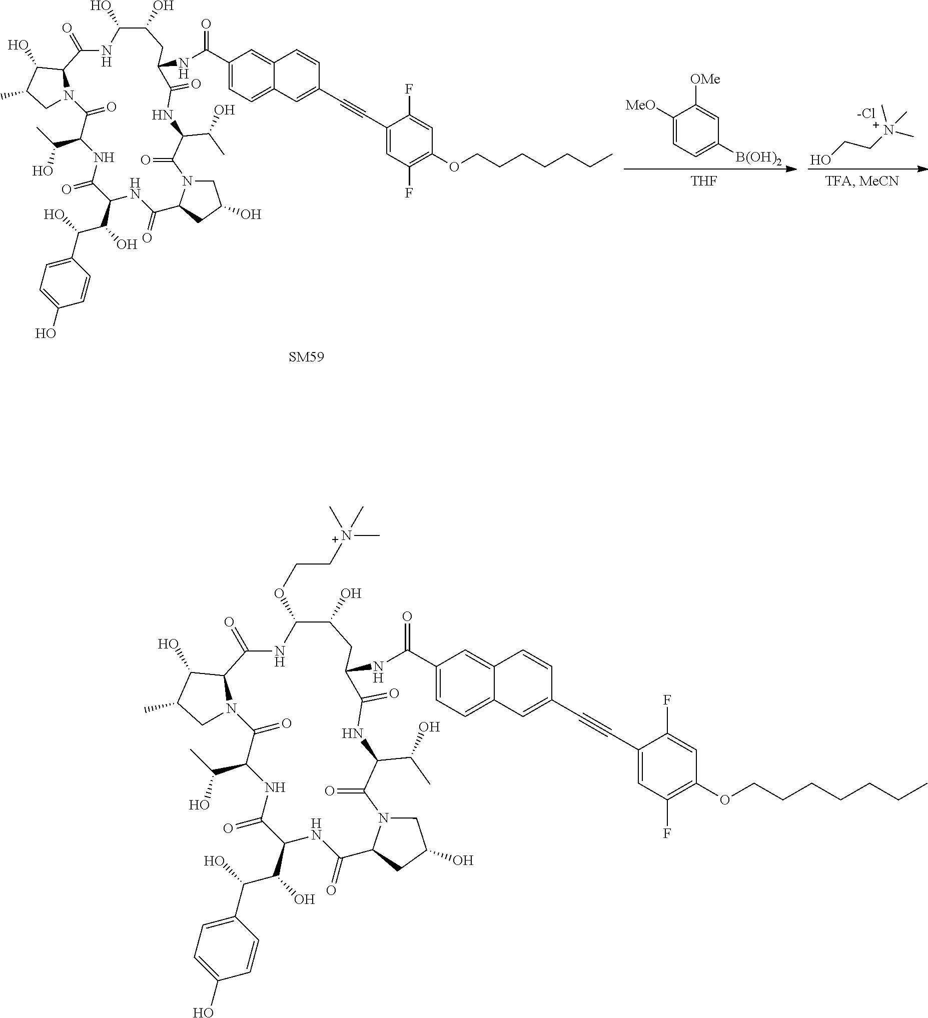

SM59 (144 mg, 0.11 mmol, 1 eq.) was dissolved in dry THF (3 mL), and 3,4-dimethoxyphenylboronic acid (28 mg, 0.15 mmol, 1.3 eq.) was added. The resulting mixture was stirred for 1 h at room temperature, the solvent was removed, and then dry THF (7 mL) was added for rotary evaporation. Choline chloride (167 mg, 1.19 mmol, 10 eq.) was added, and TFA (0.36 mL) mixed with acetonitrile (3 mL) was added to the system. The system was stirred for 3 h at room temperature. LCMS showed the formation of a product. 21 mg of a compound (hydrochloride) as a white solid was obtained through prep-HPLC purification, with a purity of 97% and a yield of 13%. HRMS[M]⁺: 1287.5902.

¹H NMR (400 MHz, CD₃OD) δ 8.43 (s, 1H), 8.10 (s, 1H), 7.98-7.91 (m, 3H), 7.63 (dd, J=8.5, 1.3 Hz, 1H), 7.32 (dd, J=11.0, 6.7 Hz, 1H), 7.15 (d, J=8.5 Hz, 2H), 7.02 (dd, J=10.8, 7.2 Hz, 1H), 6.76 (d, J=8.5 Hz, 2H), 5.44-5.37 (m, 1H), 5.06 (dd, J=8.3, 3.0 Hz, 1H), 4.79 (dd, J=12.1, 4.9 Hz, 1H), 4.59 (dd, J=11.5, 6.8 Hz, 3H), 4.39 (d, J=4.2 Hz, 1H), 4.37-4.31 (m, 2H), 4.25 (dd, J=11.6, 3.3 Hz, 2H), 4.22-4.17 (m, 1H), 4.12 (d, J=8.7 Hz, 1H), 4.08 (t, J=6.4 Hz, 2H), 3.99 (d, J=7.8 Hz, 2H), 3.95-3.88 (m, 2H), 3.82 (d, J=10.8 Hz, 1H), 3.61 (dd, J=11.9, 7.3 Hz, 1H), 3.55-3.45 (m, 2H), 3.12 (s, 9H), 2.54-2.41 (m, 2H), 2.29 (t, J=8.9 Hz, 1H), 2.08 (dd, J=12.0, 8.7 Hz, 2H), 1.82 (dd, J=14.6, 6.6 Hz, 2H), 1.51 (dd, J=14.8, 7.1 Hz, 2H), 1.42-1.32 (m, 6H), 1.27 (d, J=6.1 Hz, 6H), 1.07 (d, J=6.9 Hz, 3H), 0.92 (t, J=6.8 Hz, 3H).

### Example 22

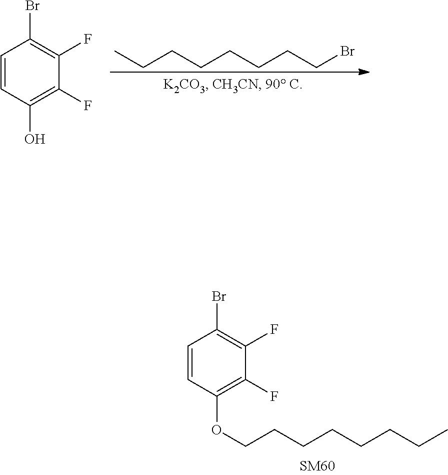

4-Bromo-2,3-difluorophenol (2 g, 9.50 mmol, 1 eq.), 1-bromooctane (1.98 ml, 11.00 mmol, 1.2 eq.) and potassium carbonate (4 g, 28.00 mmol, 3 eq.) were dissolved in acetonitrile (47 mL), and the mixture was heated, refluxed, and stirred at 90° C. After 1.5 h, TLC showed the formation of a new spot and the disappearance of the raw materials. The solvent was removed, ethyl acetate and water were added for extraction, the organic phases were combined, dried with anhydrous sodium sulfate, and concentrated to obtain 3 g of compound SM60 as a black oil, with a yield of 97%.

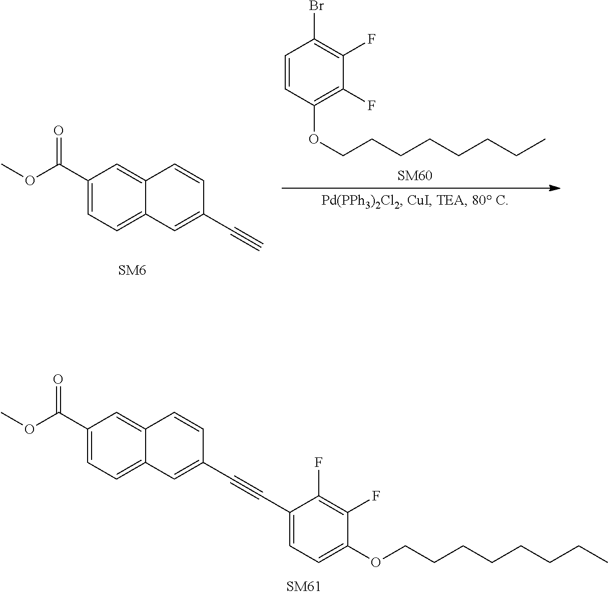 [SMILES: C#Cc1ccc2cc(C(=O)OC)ccc2c1 ; CCCCCCCCOc1ccc(Br)c(F)c1F ; CCCCCCCCOc1ccc(C#Cc2ccc3cc(C(=O)OC)ccc3c2)c(F)c1F]

SM6 (2 g, 9.50 mmol, 1 eq.), SM60 (3 g, 9.50 mmol, 1 eq.) and CuI (550 mg, 0.47 mmol, 0.05 eq.) were dissolved in 1,4-dioxane (10 mL), and then DIPEA (3.3 mL, 19.00 mmol, 3 eq.) was added. Vacuumizing and replacement with nitrogen were performed and repeated three times. Pd(PPh₃)₂Cl₂ (550 mg, 0.47 mmol, 0.05 eq.) was added under nitrogen atmosphere. The resulting mixture was heated and stirred overnight at 80° C. TLC showed a clear new spot. The solvent was removed. DCM was added for dissolving, and column chromatography purification was performed to obtain 681 mg of compound SM61 as a white solid, with a yield of 16%.

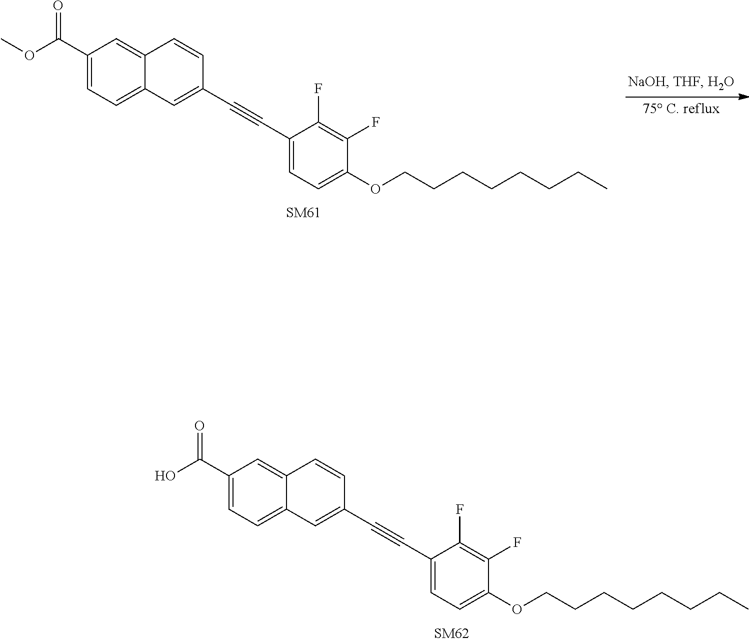 [SMILES: CCCCCCCCOc1ccc(C#Cc2ccc3cc(C(=O)OC)ccc3c2)c(F)c1F ; CCCCCCCCOc1ccc(C#Cc2ccc3cc(C(=O)O)ccc3c2)c(F)c1F]

SM61 (681 mg, 1.50 mmol, 1 eq.) was dissolved in THF (6 mL), and heated and stirred at 60° C., and an aqueous solution (1 mL) of NaOH (242 mg, 6.00 mmol, 5 eq.) was added to the reaction system. The reaction system was heated at 75° C. and refluxed overnight. After the reaction was completed, 2M HCl (aq.) was added to adjust the pH value until an acidic state is reached, and then a solid was precipitated. Suction filtration was performed to obtain 595 mg of compound SM62 as a white solid, with a yield of 89%.

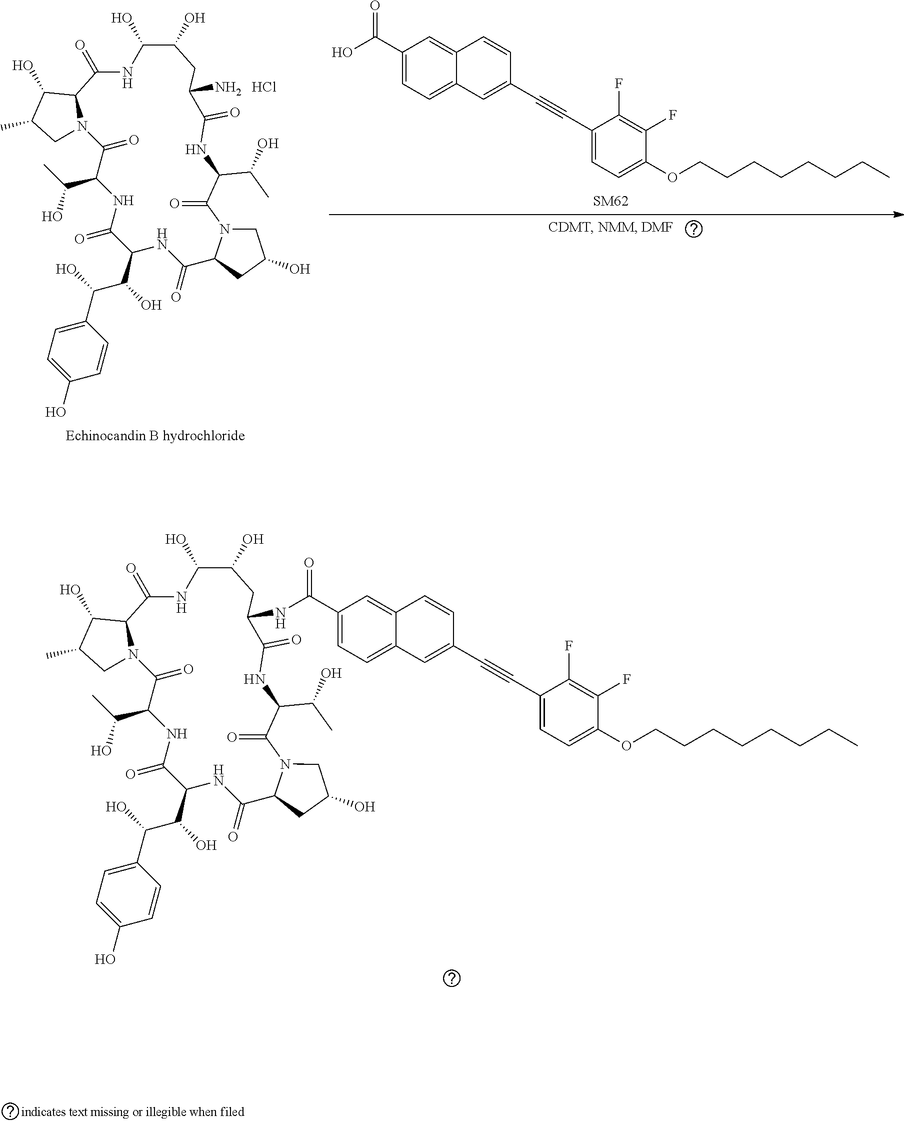

Echinocandin B hydrochloride (600 mg, 0.71 mmol, 1 eq.), SM62 (315 mg, 0.71 mmol, 1 eq.) and CDMT (40 mg, 0.22 mmol, 1.2 eq.) were dissolved in DMF (2 mL), and then NMM (0.06 mL, 0.57 mmol, 3 eq.) was added. Stirring was performed for 1 h at room temperature. After the reaction was completed, the reaction solution was subjected to prep-HPLC purification and separation to obtain 579 mg of compound SM63 as a white solid, with a purity of 96% and a yield of 57%. MS[M+H]⁺: 1216.

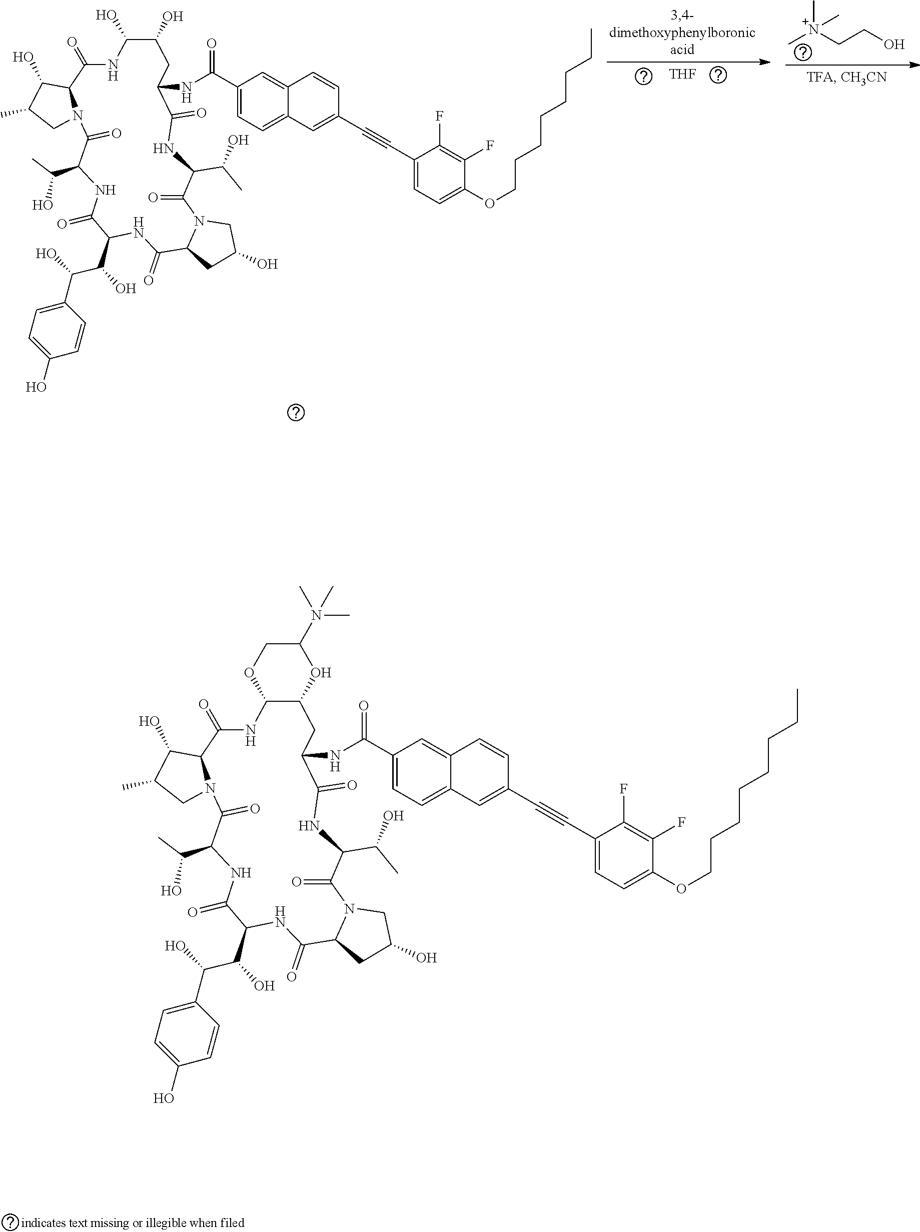

SM63 (337 mg, 0.27 mmol, 1 eq.) and 3,4-dimethoxyphenylboronic acid (65 mg, 0.36 mmol, 1.3 eq.) were dissolved in dry THF (4 mL). The resulting solution was stirred for 1 h at room temperature, and concentrated to dryness. Choline tosylate (2.3 g, 8.30 mmol, 30 eq.) was added, then a mixed solution of TFA (0.85 mL) and acetonitrile (4 mL) was added to dissolve the reactants, and stirring was performed for 6 h at room temperature under N₂ atmosphere. After the reaction was completed, an aqueous solution of sodium acetate was added for quenching, and 197 mg of a compound (acetate) as a white solid was obtained through prep-HPLC purification, with a purity of 95% and a yield of 54%. HRMS[M]⁺: 1301.6172.

¹H NMR (400 MHz, CD₃OD) δ 8.46 (s, 1H), 8.14 (s, 1H), 8.04-7.97 (m, 3H), 7.66 (dd, J=8.4, 1.4 Hz, 1H), 7.34-7.30 (m, 1H), 7.15 (d, J=8.5 Hz, 2H), 6.97 (d, J=7.5 Hz, 1H), 6.76 (d, J=8.6 Hz, 2H), 5.42 (d, J=2.4 Hz, 1H), 5.04 (d, J=3.2 Hz, 1H), 4.79 (dd, J=12.0, 5.1 Hz, 2H), 4.61-4.56 (m, 3H), 4.39 (d, J=4.3 Hz, 1H), 4.33 (d, J=8.2 Hz, 2H), 4.27-4.22 (m, 2H), 4.20-4.16 (m, 1H), 4.13 (t, J=6.4 Hz, 3H), 4.00 (d, J=11.4 Hz, 1H), 3.94-3.87 (m, 2H), 3.83 (d, J=11.4 Hz, 1H), 3.60 (d, J=4.9 Hz, 1H), 3.52-3.46 (m, 2H), 3.13 (s, 9H), 2.52-2.43 (m, 2H), 2.30 (t, J=9.0 Hz, 1H), 2.08 (dd, J=15.4, 9.4 Hz, 2H), 1.89 (s, 3H), 1.86-1.81 (m, 2H), 1.50 (d, J=8.0 Hz, 2H), 1.35 (d, J=16.1 Hz, 8H), 1.29-1.26 (m, 6H), 1.08 (d, J=6.9 Hz, 3H), 0.91 (t, J=6.9 Hz, 3H).

### Example 23

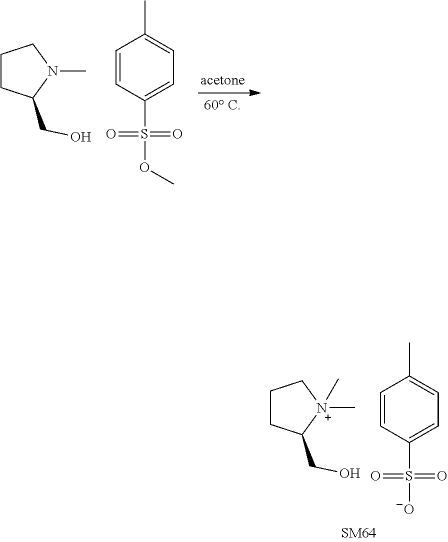

N-methyl-D-prolinol (3 g, 26.04 mmol, 1 eq.) was dissolved in acetone (30 mL). Methyl p-toluenesulfonate (4.851 g, 26.04 mmol, 1 eq.) was slowly added. The resulting mixture was refluxed for 3 h at 60° C. LCMS showed that most of the substances were products. Solids were precipitated by adding petroleum ether, and suction filtration was performed. The filter cake was washed with petroleum ether and dried to obtain 6.69 g of product SM64, with a yield of 85%.

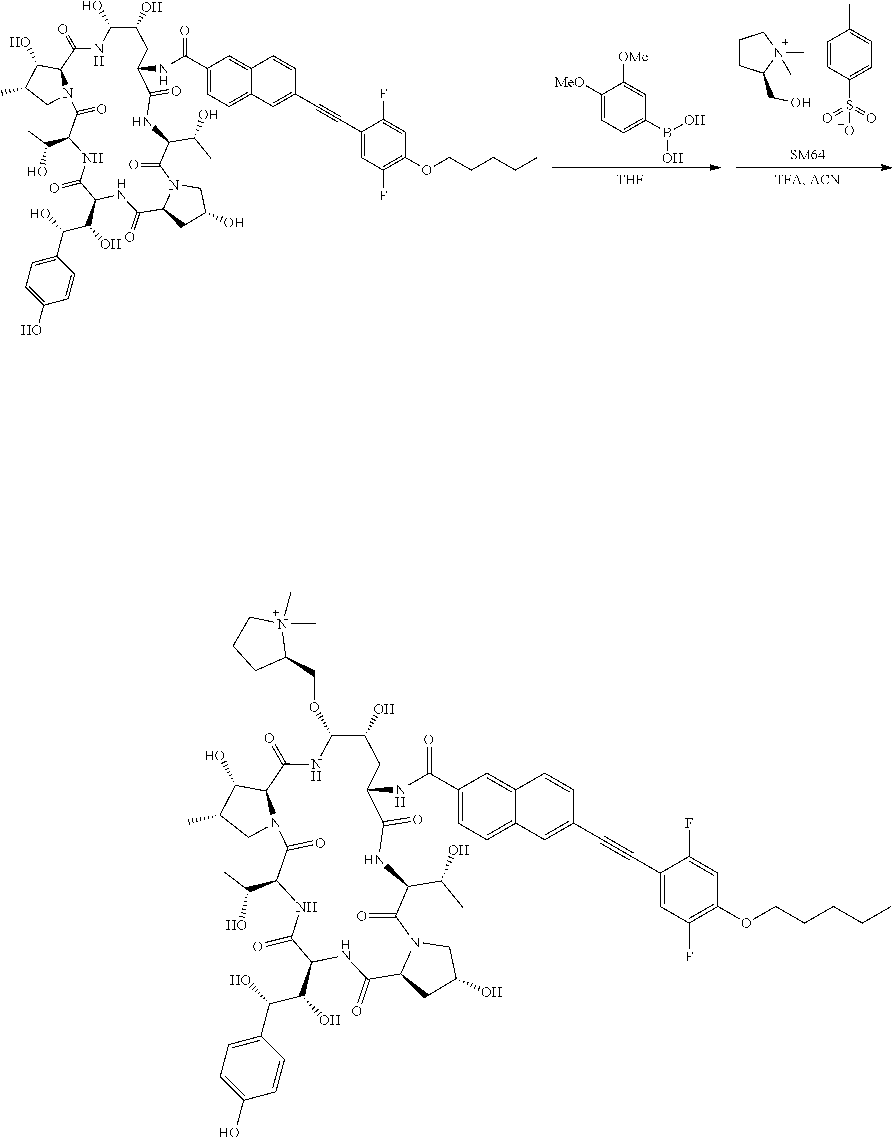

Example 15 (300 mg, 0.25 mmol, 1 eq.) was dissolved in dry THF (3 mL), and 3,4-dimethoxyphenylboronic acid (60 mg, 0.33 mmol, 1.3 eq.) was added. After the resulting mixture was stirred for 1 h at room temperature, the solvent was subjected to rotary evaporation, and then dry THF (3 mL) was added for rotary evaporation. SM64 (2.308 g, 7.66 mmol, 30 eq.) was added, and TFA (0.75 mL) mixed with dry MeCN (3 mL) was added to the system. The system was stirred for 3 h at room temperature. LCMS showed the formation of a product. 40 mg of a compound (hydrochloride) as a white solid was obtained through prep-HPLC purification, with a purity of 88% and a yield of 12%. HRMS[M]⁺: 1285.5715.

¹H NMR (400 MHz, CD₃OD) δ 8.45 (s, 1H), 8.12 (s, 1H), 8.03-7.95 (m, 3H), 7.65 (d, J=8.6 Hz, 1H), 7.33 (dd, J=11.0, 6.7 Hz, 1H), 7.15 (d, J=8.5 Hz, 2H), 7.02 (dd, J=10.7, 7.2 Hz, 1H), 6.76 (d, J=8.5 Hz, 2H), 5.42 (d, J=6.8 Hz, 1H), 5.04 (d, J=5.3 Hz, 1H), 4.80 (s, 1H), 4.60 (d, J=12.0 Hz, 3H), 4.39 (d, J=4.2 Hz, 1H), 4.34 (d, J=8.0 Hz, 2H), 4.27 (dd, J=13.1, 6.3 Hz, 2H), 4.21-4.17 (m, 1H), 4.09 (t, J=6.4 Hz, 3H), 4.00 (d, J=8.8 Hz, 2H), 3.87 (dt, J=16.9, 9.4 Hz, 4H), 3.62-3.46 (m, 3H), 3.20 (s, 3H), 2.99 (s, 3H), 2.55-2.41 (m, 2H), 2.34-2.21 (m, 2H), 2.14-2.03 (m, 4H), 1.97-1.89 (m, 1H), 1.87-1.80 (m, 2H), 1.51-1.40 (m, 4H), 1.27 (dd, J=6.1, 3.3 Hz, 6H), 1.08 (d, J=6.9 Hz, 3H), 0.97 (t, J=7.1 Hz, 3H). Example 24:

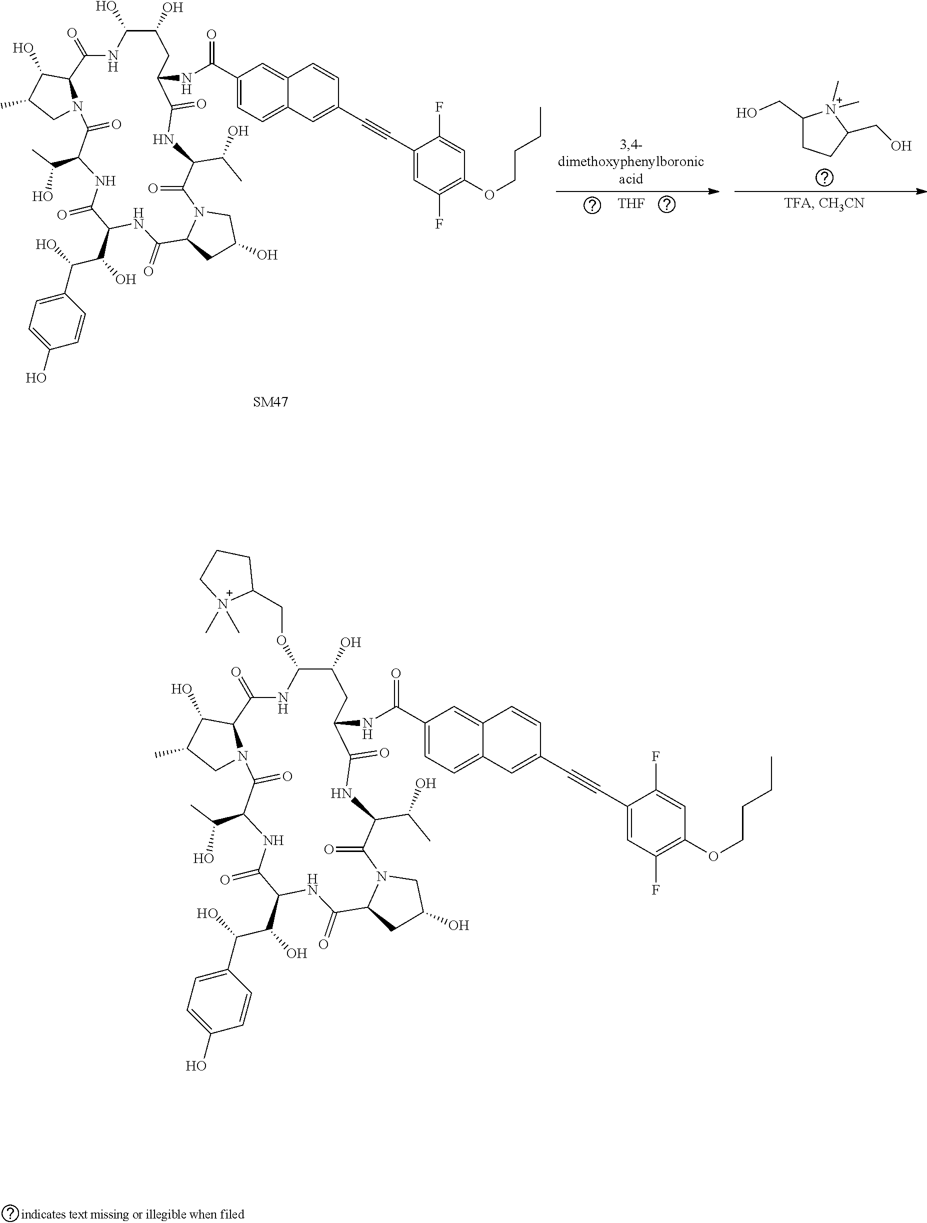

SM47 (50 mg, 0.04 mmol, 1 eq.) and 3,4-dimethoxyphenylboronic acid (10 mg, 0.05 mmol, 1.3 eq.) were dissolved in dry THF (1 mL). The resulting solution was stirred for 1 h at room temperature, and concentrated to dryness. SM64 (389 mg, 1.2 mmol, 30 eq.) was added, then a mixed solution of TFA (0.1 mL) and CH₃CN (2 mL) was added to dissolve the reactants, and stirring was performed for 5 h at room temperature under N₂ atmosphere. After the reaction was completed, an aqueous solution of sodium acetate was added for quenching, and 25 mg of a compound (hydrochloride) as a white solid was obtained through prep-HPLC purification, with a purity of 90% and a yield of 45%. HRMS[M]⁺: 1271.5322.

¹H NMR (400 MHz, CD₃OD) δ 8.46 (s, 1H), 8.13 (s, 1H), 8.05-7.94 (m, 3H), 7.65 (dd, J=8.5, 1.5 Hz, 1H), 7.33 (dd, J=11.0, 6.7 Hz, 1H), 7.15 (d, J=8.6 Hz, 2H), 7.03 (dd, J=10.8, 7.2 Hz, 1H), 6.76 (d, J=8.6 Hz, 2H), 5.41 (s, 1H), 5.07-5.01 (m, 1H), 4.79 (dd, J=11.8, 5.2 Hz, 2H), 4.59 (dd, J=10.8, 7.4 Hz, 3H), 4.39 (d, J=4.3 Hz, 1H), 4.36-4.30 (m, 2H), 4.30-4.22 (m, 2H), 4.21-4.15 (m, 1H), 4.10 (t, J=6.4 Hz, 3H), 4.00 (d, J=8.4 Hz, 2H), 3.93-3.79 (m, 4H), 3.67-3.41 (m, 4H), 3.20 (s, 3H), 2.99 (s, 3H), 2.54-2.41 (m, 2H), 2.34-2.20 (m, 2H), 2.09 (t, J=10.7 Hz, 3H), 1.97-1.88 (m, 1H), 1.82 (dt, J=12.4, 6.4 Hz, 2H), 1.54 (dq, J=14.8, 7.4 Hz, 2H), 1.27 (dd, J=6.2, 3.0 Hz, 6H), 1.08 (d, J=6.9 Hz, 3H), 1.01 (t, J=7.4 Hz, 3H).

### Experimental Example 1: Method for Testing Antifungal Activity

After gradient dilution of test compounds, an MIC assay was performed on a Candida standard strain, and an MEC assay was performed on an Aspergillus standard strain. The method for assaying minimum inhibitory concentration (MIC) was performed with reference to American Clinical and Laboratory Standards Institute (CLSI M27-A3) guidelines, and the method for assaying minimum effective concentration (MEC) was performed with reference to American Clinical and Laboratory Standards Institute (CLSI M38-A2) guidelines.

### Preparation of Fungal Inoculation Solution

### Candida:

A cryopreserved strain was passaged at least 2 times. A single colony was picked and resuspended in a physiological saline or sterile water tube, and the tube was subjected to vortex shaking. The Candida suspension was adjusted to 0.5 McF (1×10⁶-5×10⁶ CFU/mL) at a wavelength of 530 nm by using a spectrophotometer. After being diluted 50 times with physiological saline, the Candida suspension was diluted 20 times with 1×RPMI 1640 broth (1×10³-5×10³ CFU/mL). 10 μL of the diluted Candida suspension was coated on an SDA plate for colony counting, with a range of about 10-50 single colonies.

After a prepared drug sensitive plate was completely dissolved at room temperature, the Candida suspension was added to the 96-well plate by using a multi-channel pipettor at 100 μL/well. At this time, the concentration of Candida in each well should be 0.5×10³-2.5×10³ CFU/mL.

Aspergillus (Operations were Performed in a Class II Biosafety Cabinet):

Aspergillus was passaged onto an SDA plate for cultivation for 48 h-7 d at 35° C. to induce sporulation. The colonies on the plate were covered with 0.85% physiological saline (1 mL) or sterile water (polysorbate 20 was added to a final concentration of 0.1%-0.01%). The surface of the culture medium was gently wiped with a sterile cotton swab (note: do not puncture the culture medium). The resuspension of spore hyphae was transferred to a sterile test tube, and left to stand for 3-5 min to precipitate heavier particles. The homogeneous suspension on the upper layer was transferred to a new sterile test tube, and the tube was capped closely, and subjected to vortex shaking for 15 s (note: the suspension can produce an aerosol when the cap is reopened). The concentration of the suspension was adjusted to an OD value of 0.09-0.13 at 530 nm by using a spectrophotometer. The suspension was diluted 50 times with 1×RPMI 1640. Within 2 h after dilution, 100 μL of the diluted suspension was added to each well in a 96-well plate (the concentration of spores in the final drug sensitive plate should be 0.4×10⁴-5×10⁴ CFU/mL).

Colony counting: The suspension after dilution with RPMI 1640 was further diluted 10 times, 10 μL of the diluted suspension was coated on an SDA plate, cultivation was performed at 28° C., observation was performed every day, and counting was immediately performed after the colonies were visible to naked eyes.

### Cultivation

A yeast-type fungus assay plate was placed in an incubator, and incubated for 24 h at 35° C. and 85% humidity, and then the MIC values were read. For echinocandin-based drugs, Aspergillus was incubated for 21-26 h at 28° C., and then the MEC results were read.

### MIC or MEC Interpretation

Yeast-type fungi: A 96-well plate was attached with a disposable parafilm, shaken for uniformly mixing, and observed with naked eyes by using a plate reader microscope. Compared with a growth control, the minimum compound concentration corresponding to >50% growth inhibition was defined as MIC. Pictures were taken and saved by using an automated plate reader.

Aspergillus: For echinocandin-based drugs, compared to a growth control, the minimum drug concentration at which hyphae were able to form small, round and compact hyphal particles under a plate reader microscope was defined as MEC. In order to accurately determine MEC value, vortex oscillation should not be allowed before reading the plate.

TABLE 1
Test results of antifungal activity of compounds

| | | Candida | Candida | C. | C. | Candida | Candida |
|---|---|---|---|---|---|---|---|
| | Starting assay | albicans | albicans | parapsilosis | krusei | albicans | albicans |
| | concentration | ATCC | ATCC | ATCC | ATCC | ATCC | ATCC |
| Compound | (μg/mL) | 90028 | 10231 | 22019 | 6258 | MYA-574 | 64124 |
| Caspofungin | 16 | 0.12 | 0.12 | 0.5 | 0.25 | ≤0.015 | 0.12 |
| Rezafungin | 16 | ≤0.015 | ≤0.015 | 0.5 | 0.03 | ≤0.015 | ≤0.015 |
| Example 8 | 16 | ≤0.015 | 0.03 | 0.5 | 0.12 | ≤0.015 | ≤0.015 |
| Example 10 | 16 | ≤0.015 | 0.03 | 0.5 | 0.06 | ≤0.015 | ≤0.015 |
| Example 14 | 16 | ≤0.015 | 0.03 | 0.25 | 0.06 | ≤0.015 | ≤0.015 |
| Example 16 | 16 | 0.03 | 0.03 | 0.5 | 0.06 | ≤0.015 | ≤0.015 |
| Example 18 | 16 | 0.06 | 0.06 | 2 | 0.5 | ≤0.015 | 0.06 |
| Example 19 | 16 | ≤0.015 | 0.03 | 1 | 0.12 | ≤0.015 | 0.03 |

Notes:
1. C. parapsilosis ATCC 22019 and C. krusei ATCC 6258 are quality-control strains. According to CLSI-M60, the 24 h MIC of caspofungin against ATCC 22019 is 0.25-1 μg/mL, and the 24 h MIC of caspofungin against ATCC 6258 is 0.12-1 μg/mL.

TABLE 2
Test results of antifungal activity of compounds

| | | Candida | Candida | | | Aspergillus |
|---|---|---|---|---|---|---|
| | Starting assay | albicans | albicans | C. tropicalis | C. glabrata | fumigatus |
| | concentration | ATCC | ATCC | ATCC | ATCC | ATCC |
| Compound | (μg/mL) | MYA-2876 | MYA-427 | 750 | 15126 | 204305 |
| Caspofungin | 16 | 0.06 | 0.12 | 0.06 | 0.12 | 0.25 |
| Rezafungin | 16 | ≤0.015 | ≤0.015 | ≤0.015 | 0.06 | ≤0.015 |
| Example 8 | 16 | ≤0.015 | 0.03 | ≤0.015 | 0.06 | 0.03 |
| Example 10 | 16 | ≤0.015 | 0.03 | ≤0.015 | 0.06 | ≤0.015 |
| Example 14 | 16 | ≤0.015 | 0.03 | ≤0.015 | 0.06 | ≤0.015 |
| Example 16 | 16 | ≤0.015 | 0.03 | ≤0.015 | 0.06 | ≤0.015 |
| Example 18 | 16 | 0.03 | 0.12 | ≤0.015 | 0.12 | 0.03 |
| Example 19 | 16 | ≤0.015 | 0.03 | ≤0.015 | 0.12 | 0.03 |

Test results of antifungal activity of compounds

| | | Candida | Candida | Candida | Candida | Candida |
|---|---|---|---|---|---|---|
| | Starting assay | albicans | albicans | albicans | albicans | albicans |
| | concentration | ATCC | ATCC | ATCC | ATCC | ATCC |
| Compound | (μg/mL) | 76485 | MYA-4788 | MYA-1023 | 90029 | 18804 |
| Caspofungin | 16 | 0.06 | 0.06 | 0.06 | 0.06 | 0.06 |
| Rezafungin | 16 | ≤0.015 | ≤0.015 | ≤0.015 | ≤0.015 | ≤0.015 |
| Example 8 | 16 | ≤0.015 | ≤0.015 | ≤0.015 | ≤0.015 | ≤0.015 |
| Example 10 | 16 | ≤0.015 | ≤0.015 | ≤0.015 | ≤0.015 | ≤0.015 |
| Example 14 | 16 | ≤0.015 | ≤0.015 | ≤0.015 | ≤0.015 | ≤0.015 |
| Example 16 | 16 | ≤0.015 | ≤0.015 | ≤0.015 | ≤0.015 | ≤0.015 |
| Example 18 | 16 | 0.06 | 0.06 | 0.03 | 0.03 | 0.03 |
| Example 19 | 16 | 0.03 | 0.03 | ≤0.015 | ≤0.015 | ≤0.015 |

TABLE 4
Test results of antifungal activity of compounds

| | | Candida | Candida | Candida | Pichia | Cryptococcus |
|---|---|---|---|---|---|---|
| | Starting assay | albicans | albicans | albicans | guilliermondii | neoformans |
| | concentration | CMCC | ATCC | CICC | ATCC | ATCC |
| Compound | (μg/mL) | 98001 | 14053 | 1943 | 6260 | 208821 |
| Caspofungin | 16 | 0.06 | 0.03 | 0.06 | 0.12 | 16 |
| Rezafungin | 16 | ≤0.015 | ≤0.015 | ≤0.015 | 0.25 | 16 |
| Example 8 | 16 | ≤0.015 | ≤0.015 | 0.03 | 0.25 | 8 |
| Example 10 | 16 | ≤0.015 | ≤0.015 | 0.03 | 0.25 | 8 |
| Example 14 | 16 | ≤0.015 | ≤0.015 | 0.03 | 0.25 | 8 |
| Example 16 | 16 | ≤0.015 | ≤0.015 | 0.03 | 0.25 | 8 |
| Example 18 | 16 | 0.03 | 0.03 | 0.06 | 0.5 | >16 |
| Example 19 | 16 | ≤0.015 | ≤0.015 | 0.03 | 0.5 | 16 |

### Experimental Example 2: Study on Pharmacokinetic Properties in Rats

The pharmacokinetic properties of rezafungin, example 16 and example 19 in the blood plasma of SPF-grade SD rats (as test animals) after intravenous injection were studied.

TABLE 5
Experimental scheme of study on pharmacokinetic properties in rats

| | Number | | | | Administration | |
|---|---|---|---|---|---|---|
| | of | | Dosage | Concentration | volume | Collection time |
| Group | animals | Test sample | (mg/kg) | (mg/mL) | (mL/kg) | point |
| A | 4 | Rezafungin | 5 | 0.67 | 7.5 | 0.17 h, 6 h, 22 h, |
| | | Example 16 | 5 | 0.67 | 7.5 | 30 h, 46 h, 54 h, |
| | | | | | | 70 h post- |
| | | | | | | administration |
| B | 3 | Example 16 | 2.5 | 0.33 | 7.5 | 0.33 h, 4 h, 10 h, |
| | | Example 19 | 2.5 | 0.33 | 7.5 | 24 h, 31 h, 48 h, |
| | | | | | | 57.5 h, 72 h post- |
| | | | | | | administration |

The pharmacokinetic parameters are as shown in the table below

TABLE 6
Pharmacokinetic parameters in rats

| | | T_{1/2} | Cₘₐₓ | AUC₀₋ₜ | AUC_{0-inf} | CL | MRT₀₋ₜ | V_{z} |
|---|---|---|---|---|---|---|---|---|
| Group | Test sample | h | mg/L | mg/L × h | mg/L × h | L/h/kg | h | L/kg |
| A | Rezafungin | 27.5 | 4.75 | 89 | 104 | 0.048 | 20.2 | 1.904 |
| | Example 16 | 34.8 | 11.95 | 326 | 424 | 0.012 | 24.2 | 0.591 |
| B | Example 16 | 37.3 | 6.08 | 206 | 275 | 0.009 | 26.1 | 0.49 |
| | Example 19 | 46.4 | 9.62 | 379 | 554 | 0.005 | 27.5 | 0.302 |

The experimental data showed that the drug exposure level (Cmax and AUC) and half-life (T_{1/2}) of example 16 and example 19 in the blood plasma after single intravenous administration were significantly higher than those of rezafungin at the same dose, providing a new choice for clinical medication.

What is claimed is:

1. A compound as shown in formula I, or a pharmaceutically acceptable salt thereof or an isomer thereof, wherein: [symbol]
X, Y and Z are each independently selected from C or N;
R₂, R₃, R₄, R₅, R₆, R₈, R₉, R₁₀, R₁₁ and R₁₂ are each independently selected from hydrogen, deuterium, halogen, cyano, thiocyano, isothiocyano or C₁₋₁₀ lower alkyl;
R₇ is selected from C₁₋₁₀ lower alkyl, C₂₋₁₀ alkenyl, C₂₋₁₀ alkynyl, aryl, heteroaryl, cyclic hydrocarbonyl or heterocyclyl;
R₁ is selected from hydroxyl, hydrogen, deuterium, halogen, cyano, thiocyano, isothiocyano, O[C(R_{A1})(R_{A2})]ₐ[C(R_{A3})(R_{A4})]ⱼX₁, NH[C(R_{A1})(R_{A2})]ₐ[C(R_{A3})(R_{A4})]ⱼX₁, O(CH₂CH₂O)_{b}CH₂CH₂X₁, O(CH₂CH₂CH₂O)_{b}CH₂CH₂X₁, O(CH₂CH₂NH)_{b}CH₂CH₂X₁, NH(CH₂CH₂O)_{b}CH₂CH₂X₁, NH(CH₂CH₂NH)_{b}CH₂CH₂X₁, NH(CH₂CH₂CH₂O)_{b}CH₂CH₂X₁, NH[(CH₂(CH₂)_{c}O)]_{b}CH{CH₂[OCH₂(CH₂)_{c}]_{d}X₁}₂, O[(CH₂(CH₂)_{c}O)]_{b}CH{CH₂[OCH₂(CH₂)_{c}]_{d}X₁}₂ or (OCH₂CH₂)_{b}(NHCH₂CH₂)ₑX₂,
R_{A1}, R_{A2}, R_{A3} and R_{A4} are independently selected from hydrogen, deuterium, halogen, C₁₋₁₀ lower alkyl, cyclic hydrocarbonyl or cyclic hydrocarbylene [symbol] ,
X₁ is independently N(R_{C1}R_{C2}R_{C3}) or the following structure [symbol] ;
ring A is an optionally substituted, saturated or unsaturated, monocyclic ring or fused ring containing one or more N atoms;
R_{C1}, R_{C2} and R_{C3} are independently selected from H, C₁₋₆ alkyl, halogenated C₁₋₆ lower alkyl and deuterated C₁₋₆ lower alkyl, and at least one of R_{C1}, R_{C2} or R_{C3} is not hydrogen,
each R_{F} is independently selected from H, deuterium, hydroxyl, hydroxylalkyl, amino, alkoxy, lower alkyl, alkenyl, alkynyl, halogen, SR′, SOR′, SO₂R′, NR′(R″), COOR′ and CONR′(R″), wherein the lower alkyl is optionally substituted with one or more substituents selected from deuterium, alkyl, cycloalkyl, alkoxy, hydroxylalkyl, alkenyl, alkynyl, aryl, heteroaryl, nitro, a nitrile group, hydroxyl, halogen, SR′, NR′(R″), COOR′ or CONR′(R″);
X₂ is N(R_{D1}R_{D2}R_{D3}) or the structure of X₁;
R_{D1}, R_{D2} and R_{D3} are independently selected from H, C₁₋₆ lower alkyl, halogenated C₁₋₆ lower alkyl or deuterated C₁₋₆ lower alkyl;
R′ and R″ are independently selected from hydrogen, hydroxyl, alkyl, alkoxy, alkenyl or —C(O)R_{J};
R_{J} is selected from hydrogen, deuterium, C₁₋₁₀ lower alkyl, cyclic hydrocarbonyl or cyclic hydrocarbylene;
and a is an integer of 0-5, b is an integer of 1-5, c is an integer of 1-2, d is an integer of 0-3, e is an integer of 1-5, k is an integer of 0-20, j is an integer of 0-5, and n is an integer of 1-7.

2. The compound, or the pharmaceutically acceptable salt thereof or the isomer thereof according to claim 1, wherein
R₁ is selected from O(C(R_{A1})(R_{A2}))ₐ(C(R_{A3})(R_{A4}))ⱼX₁, NH(C(R_{A1})(R_{A2}))ₐ(C(R_{A3})(R_{A4}))ⱼX₁, O(CH₂CH₂O)_{b}CH₂CH₂X₁, O(CH₂CH₂CH₂O)_{b}CH₂CH₂X₁, O(CH₂CH₂NH)_{b}CH₂CH₂X₁, NH(CH₂CH₂O)_{b}CH₂CH₂X₁, NH(CH₂CH₂NH)_{b}CH₂CH₂X₁, NH(CH₂CH₂CH₂O)_{b}CH₂CH₂X₁, NH[(CH₂(CH₂)_{c}O)]_{b}CH{CH₂[OCH₂(CH₂)_{c}]_{d}X₁}₂, O[(CH₂(CH₂)_{c}O)]_{b}CH{CH₂[OCH₂(CH₂)]_{d}X₁}₂ or (OCH₂CH₂)_{b}(NHCH₂CH₂)ₑX₂,
R_{A1}, R_{A2}, R_{A3} and R_{A4} are independently selected from hydrogen, deuterium, halogen, C₁₋₁₀ lower alkyl, cyclic hydrocarbonyl or cyclic hydrocarbylene, [symbol]
X₁ is independently N(R_{C1}R_{C2}R_{C3}) or the following structure [symbol] :
Ring A is an optionally substituted, saturated or unsaturated, monocyclic ring or fused ring containing one or more N atoms;
R_{C1}, R_{C2} and R_{C3} are independently selected from H, halogenated C₁₋₆ lower alkyl and deuterated C₁₋₆ lower alkyl, and at least one of R_{C1}, R_{C2} or R_{C3} is not hydrogen;
each R_{F} is independently selected from H, deuterium, hydroxyl, hydroxylalkyl, amino, alkoxy, lower alkyl, alkenyl, alkynyl, halogen, SR′, SOR′, SO₂R′, NR′(R″), COOR′ or CONR′(R″), wherein the lower alkyl is optionally substituted with one or more substituents selected from deuterium, alkyl, cycloalkyl, alkoxy, hydroxylalkyl, alkenyl or alkynyl;
X₂ is N(R_{D1}R_{D2}R_{D3}) or the structure of X₁;
R_{D1}, R_{D2} and R_{D3} are independently selected from H, C₁₋₆ lower alkyl, halogenated C₁₋₆ lower alkyl or deuterated C₁₋₆ lower alkyl;
R′ and R″ are independently selected from hydrogen, hydroxyl, alkyl, alkoxy, alkenyl or —C(O)R_{J};
R_{J} is selected from hydrogen, C₁₋₁₀ lower alkyl, cyclic hydrocarbonyl or cyclic hydrocarbylene;
and a is an integer of 0-5, b is an integer of 1-5, c is an integer of 1-2, d is an integer of 0-3, e is an integer of 1-5, k is an integer of 0-20, j is an integer of 0-5, and n is an integer of 1-7.

3. The compound as shown in formula I, or the pharmaceutically acceptable salt thereof or the isomer thereof according to claim 1, wherein
X₁ is selected from the following structures: [symbol]
wherein each R_{F} is independently selected from H, deuterium, hydroxyl, hydroxylalkyl, amino, alkoxy, lower alkyl, alkenyl, alkynyl, halogen, SR′, SOR′, SO2R′, NR′(R″), COOR′ and CONR′(R″), wherein the lower alkyl is optionally substituted with one or more substituents selected from deuterium, alkyl, cycloalkyl, alkoxy, hydroxylalkyl, alkenyl or alkynyl;
R_{q1} and R_{q2} are independently H or C₁₋₆ lower alkyl, the lower alkyl is optionally substituted with one or more substituents selected from deuterium, alkyl, cycloalkyl, alkoxy, hydroxylalkyl, alkenyl, alkynyl, aryl, heteroaryl, nitro, a nitrile group, hydroxyl, halogen, SR′, NR′(R″), COOR′ or CONR′(R″);
R′ and R″ are independently selected from hydrogen, hydroxyl, alkyl, alkoxy, alkenyl or —C(O)R_{J};
R_{J} is selected from hydrogen, deuterium, C₁₋₁₀ lower alkyl, cyclic hydrocarbonyl or cyclic hydrocarbylene;
and f is an integer of 0-16, g is an integer of 0-16, h is an integer of 0-9, i is an integer of 0-4, n is an integer of 1-7, and p is an integer of 1-3.

4. The compound as shown in formula I, or the pharmaceutically acceptable salt thereof or the isomer thereof according to claim 1, wherein R₁ is selected from hydroxyl, hydrogen, deuterium or one of the following structures: [symbol]
.

5. The compound as shown in formula I, or the pharmaceutically acceptable salt thereof or the isomer thereof according to claim 4, wherein R₁ is selected from hydroxyl, hydrogen or one of the following structures: [symbol]
, [symbol] , [symbol] , [symbol] , [symbol] , [symbol] , [symbol] , [symbol] .

6. The compound as shown in formula I, or the pharmaceutically acceptable salt thereof or the isomer thereof according to claim 4, wherein R₁ is selected from hydroxyl or one of the following structures: [symbol]
, [symbol] , [symbol] , [symbol] .

7. The compound as shown in formula I, or the pharmaceutically acceptable salt thereof or the isomer thereof according to claim 1, wherein R₇ is selected from C₃₋₆ lower alkyl.

8. The compound as shown in formula I, or the pharmaceutically acceptable salt thereof or the isomer thereof according to claim 1, wherein R₇ is selected from n-butyl or n-pentyl.

9. The compound as shown in formula I, or the pharmaceutically acceptable salt thereof or the isomer thereof according to claim 1, wherein the structural formula of the compound is one of the structures as follows: [symbol] [symbol] [symbol] [symbol] [symbol] [symbol] [symbol]
.

10. Use of the compound, or the pharmaceutically acceptable salt thereof or the isomer thereof according to any one of claims 1 to 9 in the preparation of a drug for inhibiting the growth of fungi or killing fungi.

11. Use of the compound, or the pharmaceutically acceptable salt thereof or the isomer thereof according to any one of claims 1 to 9 in the preparation of a drug for treating or preventing a fungal infection or a disease caused by a fungal infection.

12. The use according to claim 11, wherein the fungus is selected from one or more of the following genera: Candida albicans, C. parapsilosis, C. glabrata, C. guilliermondii, C. krusei, C. lusitaniae, C. tropicalis, Aspergillus fumigatus, A. flavus, A. terreus, A. niger, A. candidus, A. clavatus or A. ochraceus.

13. The use according to claim 11, wherein the disease caused by a fungal infection is selected from tinea capitis, tinea corporis, tinea pedis, onychomycosis, perionychomycosis, chromophytosis, thrush, vaginal candidiasis, respiratory candidiasis, biliary tract candidiasis, esophageal candidiasis, urinary tract candidiasis, systemic candidiasis, mucocutaneous candidiasis, aspergillosis, mucormycosis, paracoccidioidomycosis, North America blastomycosis, histoplasmosis, coccidioidomycosis, sporotrichosis, fungal rhino-sinusitis or chronic paranasal inflammation.

14. The use according to claim 11, wherein the disease caused by a fungal infection is selected from candidal bacteremia or invasive candidiasis.

15. An antifungal pharmaceutical composition, wherein the antifungal pharmaceutical composition comprises the compound, or the pharmaceutically acceptable salt thereof or the isomer thereof according to any one of claims 1 to 9.

16. Intermediate II of the compound of formula I: [symbol]
II
wherein the definitions of X, Y, Z, and R₂ to R₁₂ correspond to those in the compound of formula I;
and —C(═O)—R₁₃ constitutes carboxyl, acyl halide, an ester group and an anhydride group.

17. The intermediate II according to claim 16, wherein R₁₃ is selected from —OH, Cl, —O—C(═O)CH₃, and —R_{g1};
and R_{g1} is selected from [symbol] or [symbol] .
